SEQUENCE LISTING (147 sequences)

SEQ ID NO: 1 - protein, 19 aa, synthetic construct
  VARIANT 7: note = artificial amino acid, wherein the artificial amino acid is an oligo(alkylamino) acid of formula IIa as defined for X1
  VARIANT 13: note = artificial amino acid, wherein the artificial amino acid is an oligo(alkylamino) acid of formula IIa as defined for X1
  SITE 10: note = side chain of the lysine is linked via an amide linkage to another lysine
  VARIANT 5: note = residue may be deleted
  VARIANT 6: note = residue may be deleted
  VARIANT 8: note = residue may be deleted
  VARIANT 9: note = residue may be deleted
  VARIANT 11: note = residue may be deleted
  VARIANT 12: note = residue may be deleted
  VARIANT 14: note = residue may be deleted
  VARIANT 15: note = residue may be deleted
  VARIANT 1: note = residue may be deleted
  VARIANT 19: note = residue may be deleted
CYYYHHXHHKHHXHHYYYC

SEQ ID NO: 2 - protein, 19 aa, synthetic construct
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 14: note = artificial amino acid, wherein the artificial amino acid is an oligo(alkylamino) acid of formula IIa as defined for X2
  VARIANT 12: note = artificial amino acid, wherein the artificial amino acid is an oligo(alkylamino) acid of formula IIa as defined for X2
  VARIANT 1: note = residue may be deleted
  VARIANT 5: note = residue may be deleted
  VARIANT 7: note = residue may be deleted
  VARIANT 19: note = residue may be deleted
  VARIANT 15: note = residue may be deleted
  VARIANT 13: note = residue may be deleted
  VARIANT 11: note = residue may be deleted
  VARIANT 9: note = residue may be deleted
  SITE 10: note = side chain of the lysine is linked via an amide linkage to another lysine
CYYYHXHXHKHXHXHYYYC

SEQ ID NO: 3 - protein, 19 aa, synthetic construct
  SITE 10: note = side chain of the lysine is linked via an amide linkage to another lysine
  VARIANT 7: note = artificial amino acid, wherein the artificial amino acid is an oligo(alkylamino) acid of formula IIa as defined for X1
  VARIANT 13: note = artificial amino acid, wherein the artificial amino acid is an oligo(alkylamino) acid of formula IIa as defined for X1
  VARIANT 5: note = residue may be deleted
  VARIANT 6: note = residue may be deleted
  VARIANT 8: note = residue may be deleted
  VARIANT 9: note = residue may be deleted
  VARIANT 11: note = residue may be deleted
  VARIANT 12: note = residue may be deleted
  VARIANT 14: note = residue may be deleted
  VARIANT 15: note = residue may be deleted
CYYYHHXHHKHHXHHYYYC

SEQ ID NO: 4 - protein, 17 aa, synthetic construct
  SITE 9: note = side chain of the lysine is linked via an amide linkage to another lysine
  VARIANT 6: note = artificial amino acid, wherein the artificial amino acid is an oligo(alkylamino) acid of formula IIa as defined for X1
  VARIANT 12: note = artificial amino acid, wherein the artificial amino acid is an oligo(alkylamino) acid of formula IIa as defined for X1
  VARIANT 4: note = residue may be deleted
  VARIANT 5: note = residue may be deleted
  VARIANT 7: note = residue may be deleted
  VARIANT 8: note = residue may be deleted
  VARIANT 10: note = residue may be deleted
  VARIANT 11: note = residue may be deleted
  VARIANT 13: note = residue may be deleted
  VARIANT 14: note = residue may be deleted
YYYHHXHHKHHXHHYYY

SEQ ID NO: 5 - protein, 19 aa, synthetic construct
  SITE 10: note = side chain of the lysine is linked via an amide linkage to another lysine
  VARIANT 7: note = artificial amino acid, wherein the artificial amino acid is an oligo(alkylamino) acid of formula IIa as defined for X1
  VARIANT 13: note = artificial amino acid, wherein the artificial amino acid is an oligo(alkylamino) acid of formula IIa as defined for X1
CYYYHHXHHKHHXHHYYYC

SEQ ID NO: 6 - protein, 17 aa, synthetic construct
  SITE 9: note = side chain of the lysine is linked via an amide linkage to another lysine
  VARIANT 6: note = artificial amino acid, wherein the artificial amino acid is an oligo(alkylamino) acid of formula IIa as defined for X1
  VARIANT 12: note = artificial amino acid, wherein the artificial amino acid is an oligo(alkylamino) acid of formula IIa as defined for X1
YYYHHXHHKHHXHHYYY

SEQ ID NO: 7 - protein, 15 aa, synthetic construct
  VARIANT 6: note = artificial amino acid, wherein the artificial amino acid is an oligo(alkylamino) acid of formula IIa as defined for X1
  VARIANT 10: note = artificial amino acid, wherein the artificial amino acid is an oligo(alkylamino) acid of formula IIa as defined for X1
  SITE 8: note = side chain of the lysine is linked via an amide linkage to another lysine
CYYYHXHKHXHYYYC

SEQ ID NO: 8 - protein, 13 aa, synthetic construct
  VARIANT 5: note = artificial amino acid, wherein the artificial amino acid is an oligo(alkylamino) acid of formula IIa as defined for X1
  VARIANT 9: note = artificial amino acid, wherein the artificial amino acid is an oligo(alkylamino) acid of formula IIa as defined for X1
  SITE 7: note = side chain of the lysine is linked via an amide linkage to another lysine
YYYHXHKHXHYYY

SEQ ID NO: 9 - protein, 11 aa, synthetic construct
  VARIANT 5: note = artificial amino acid, wherein the artificial amino acid is an oligo(alkylamino) acid of formula IIa as defined for X1
  VARIANT 7: note = artificial amino acid, wherein the artificial amino acid is an oligo(alkylamino) acid of formula IIa as defined for X1
  SITE 6: note = side chain of the lysine is linked via an amide linkage to another lysine
CYYYXKXYYYC

SEQ ID NO: 10 - protein, 9 aa, synthetic construct
  SITE 5: note = side chain of the lysine is linked via an amide linkage to another lysine
  VARIANT 4: note = artificial amino acid, wherein the artificial amino acid is an oligo(alkylamino) acid of formula IIa as defined for X1
  VARIANT 6: note = artificial amino acid, wherein the artificial amino acid is an oligo(alkylamino) acid of formula IIa as defined for X1
YYYXKXYYY

SEQ ID NO: 11 - protein, 20 aa, synthetic construct
  SITE 11: note = side chain of the lysine is linked via an amide linkage to another lysine
  VARIANT 8: note = artificial amino acid, wherein the artificial amino acid is an oligo(alkylamino) acid of formula IIa as defined for X1
  VARIANT 14: note = artificial amino acid, wherein the artificial amino acid is an oligo(alkylamino) acid of formula IIa as defined for X1
  VARIANT 6: note = residue may be deleted
  VARIANT 7: note = residue may be deleted
  VARIANT 9: note = residue may be deleted
  VARIANT 10: note = residue may be deleted
  VARIANT 12: note = residue may be deleted
  VARIANT 13: note = residue may be deleted
  VARIANT 15: note = residue may be deleted
  VARIANT 16: note = residue may be deleted
  SITE 1: note = azido-lysine
KCYYYHHXHHKHHXHHYYYC

SEQ ID NO: 12 - protein, 18 aa, synthetic construct
  SITE 1: note = azido-lysine
  SITE 10: note = side chain of the lysine is linked via an amide linkage to another lysine
  VARIANT 7: note = artificial amino acid, wherein the artificial amino acid is an oligo(alkylamino) acid of formula IIa as defined for X1
  VARIANT 13: note = artificial amino acid, wherein the artificial amino acid is an oligo(alkylamino) acid of formula IIa as defined for X1
  VARIANT 5: note = residue may be deleted
  VARIANT 6: note = residue may be deleted
  VARIANT 8: note = residue may be deleted
  VARIANT 9: note = residue may be deleted
  VARIANT 11: note = residue may be deleted
  VARIANT 12: note = residue may be deleted
  VARIANT 15: note = residue may be deleted
  VARIANT 16: note = residue may be deleted
KYYYHHXHHKHHXHHYYY

SEQ ID NO: 13 - protein, 20 aa, synthetic construct
  SITE 1: note = azido-lysine
  SITE 11: note = side chain of the lysine is linked via an amide linkage to another lysine
  VARIANT 8: note = artificial amino acid, wherein the artificial amino acid is an oligo(alkylamino) acid of formula IIa as defined for X1
  VARIANT 14: note = artificial amino acid, wherein the artificial amino acid is an oligo(alkylamino) acid of formula IIa as defined for X1
KCYYYHHXHHKHHXHHYYYC

SEQ ID NO: 14 - protein, 18 aa, synthetic construct
  SITE 10: note = side chain of the lysine is linked via an amide linkage to another lysine
  VARIANT 7: note = artificial amino acid, wherein the artificial amino acid is an oligo(alkylamino) acid of formula IIa as defined for X1
  VARIANT 13: note = artificial amino acid, wherein the artificial amino acid is an oligo(alkylamino) acid of formula IIa as defined for X1
  SITE 1: note = azido-lysine
KYYYHHXHHKHHXHHYYY

SEQ ID NO: 15 - protein, 16 aa, synthetic construct
  SITE 1: note = azido-lysine
  SITE 9: note = side chain of the lysine is linked via an amide linkage to another lysine
  VARIANT 7: note = artificial amino acid, wherein the artificial amino acid is an oligo(alkylamino) acid of formula IIa as defined for X1
  VARIANT 11: note = artificial amino acid, wherein the artificial amino acid is an oligo(alkylamino) acid of formula IIa as defined for X1
KCYYYHXHKHXHYYYC

SEQ ID NO: 16 - protein, 14 aa, synthetic construct
  SITE 1: note = azido-lysine
  SITE 8: note = side chain of the lysine is linked via an amide linkage to another lysine
  VARIANT 6: note = artificial amino acid, wherein the artificial amino acid is an oligo(alkylamino) acid of formula IIa as defined for X1
  VARIANT 10: note = artificial amino acid, wherein the artificial amino acid is an oligo(alkylamino) acid of formula IIa as defined for X1
KYYYHXHKHXHYYY

SEQ ID NO: 17 - protein, 12 aa, synthetic construct
  SITE 1: note = azido-lysine
  SITE 7: note = side chain of the lysine is linked via an amide linkage to another lysine
  VARIANT 6: note = artificial amino acid, wherein the artificial amino acid is an oligo(alkylamino) acid of formula IIa as defined for X1
  VARIANT 8: note = artificial amino acid, wherein the artificial amino acid is an oligo(alkylamino) acid of formula IIa as defined for X1
KCYYYXKXYYYC

SEQ ID NO: 18 - protein, 10 aa, synthetic construct
  SITE 1: note = azido-lysine
  VARIANT 5: note = artificial amino acid, wherein the artificial amino acid is an oligo(alkylamino) acid of formula IIa as defined for X1
  VARIANT 7: note = artificial amino acid, wherein the artificial amino acid is an oligo(alkylamino) acid of formula IIa as defined for X1
  SITE 6: note = side chain of the lysine is linked via an amide linkage to another lysine
KYYYXKXYYY

SEQ ID NO: 19 - protein, 20 aa, synthetic construct
  VARIANT 5: note = residue may be deleted
  VARIANT 6: note = residue may be deleted
  VARIANT 7: note = artificial amino acid, wherein the artificial amino acid is an oligo(alkylamino) acid of formula IIa as defined for X1
  VARIANT 8: note = residue may be deleted
  VARIANT 9: note = residue may be deleted
  SITE 10: note = side chain of the lysine is linked via an amide linkage to another lysine
  VARIANT 11: note = residue may be deleted
  VARIANT 12: note = residue may be deleted
  VARIANT 13: note = artificial amino acid, wherein the artificial amino acid is an oligo(alkylamino) acid of formula IIa as defined for X1
  VARIANT 14: note = residue may be deleted
  VARIANT 15: note = residue can be deleted
  SITE 20: note = azido-lysine
CYYYHHXHHKHHXHHYYYCK

SEQ ID NO: 20 - protein, 18 aa, synthetic construct
  VARIANT 12: note = artificial amino acid, wherein the artificial amino acid is an oligo(alkylamino) acid of formula IIa as defined for X1
  VARIANT 6: note = artificial amino acid, wherein the artificial amino acid is an oligo(alkylamino) acid of formula IIa as defined for X1
  SITE 9: note = side chain of the lysine is linked via an amide linkage to another lysine
  SITE 18: note = azido-lysine
  VARIANT 4: note = residue may be deleted
  VARIANT 5: note = residue may be deleted
  VARIANT 7: note = residue may be deleted
  VARIANT 8: note = residue may be deleted
  VARIANT 10: note = residue may be deleted
  VARIANT 11: note = residue may be deleted
  VARIANT 13: note = residue may be deleted
  VARIANT 14: note = residue may be deleted
YYYHHXHHKHHXHHYYYK

SEQ ID NO: 21 - protein, 20 aa, synthetic construct
  VARIANT 7: note = artificial amino acid, wherein the artificial amino acid is an oligo(alkylamino) acid of formula IIa as defined for X1
  VARIANT 13: note = artificial amino acid, wherein the artificial amino acid is an oligo(alkylamino) acid of formula IIa as defined for X1
  SITE 10: note = side chain of the lysine is linked via an amide linkage to another lysine
  SITE 20: note = azido-lysine
CYYYHHXHHKHHXHHYYYCK

SEQ ID NO: 22 - protein, 18 aa, synthetic construct
  SITE 9: note = side chain of the lysine is linked via an amide linkage to another lysine
  VARIANT 6: note = artificial amino acid, wherein the artificial amino acid is an oligo(alkylamino) acid of formula IIa as defined for X1
  VARIANT 12: note = artificial amino acid, wherein the artificial amino acid is an oligo(alkylamino) acid of formula IIa as defined for X1
  SITE 18: note = azido-lysine
YYYHHXHHKHHXHHYYYK

SEQ ID NO: 23 - protein, 16 aa, synthetic construct
  VARIANT 6: note = artificial amino acid, wherein the artificial amino acid is an oligo(alkylamino) acid of formula IIa as defined for X1
  VARIANT 10: note = artificial amino acid, wherein the artificial amino acid is an oligo(alkylamino) acid of formula IIa as defined for X1
  SITE 8: note = side chain of the lysine is linked via an amide linkage to another lysine
  SITE 16: note = azido-lysine
CYYYHXHKHXHYYYCK

SEQ ID NO: 24 - protein, 14 aa, synthetic construct
  VARIANT 5: note = artificial amino acid, wherein the artificial amino acid is an oligo(alkylamino) acid of formula IIa as defined for X1
  SITE 7: note = side chain of the lysine is linked via an amide linkage to another lysine
  VARIANT 9: note = artificial amino acid, wherein the artificial amino acid is an oligo(alkylamino) acid of formula IIa as defined for X1
  SITE 14: note = azido-lysine
YYYHXHKHXHYYYK

SEQ ID NO: 25 - protein, 12 aa, synthetic construct
  VARIANT 5: note = artificial amino acid, wherein the artificial amino acid is an oligo(alkylamino) acid of formula IIa as defined for X1
  VARIANT 7: note = artificial amino acid, wherein the artificial amino acid is an oligo(alkylamino) acid of formula IIa as defined for X1
  SITE 6: note = side chain of the lysine is linked via an amide linkage to another lysine
  SITE 12: note = azido-lysine
CYYYXKXYYYCK

SEQ ID NO: 26 - protein, 10 aa, synthetic construct
  VARIANT 4: note = artificial amino acid, wherein the artificial amino acid is an oligo(alkylamino) acid of formula IIa as defined for X1
  VARIANT 6: note = artificial amino acid, wherein the artificial amino acid is an oligo(alkylamino) acid of formula IIa as defined for X1
  SITE 5: note = side chain of the lysine is linked via an amide linkage to another lysine
  SITE 10: note = azido-lysine
YYYXKXYYYK

SEQ ID NO: 27 - protein, 18 aa, synthetic construct
  VARIANT 7: note = artificial amino acid, wherein the artificial amino acid is an oligo(alkylamino) acid of formula IIa as defined for X1
  VARIANT 13: note = artificial amino acid, wherein the artificial amino acid is an oligo(alkylamino) acid of formula IIa as defined for X1
  SITE 10: note = side chain of the lysine is linked via an amide linkage to another lysine
  VARIANT 5: note = residue may be deleted
  VARIANT 6: note = residue may be deleted
  VARIANT 8: note = residue may be deleted
  VARIANT 9: note = residue may be deleted
  VARIANT 11: note = residue may be deleted
  VARIANT 12: note = residue may be deleted
  VARIANT 14: note = residue may be deleted
  VARIANT 15: note = residue may be deleted
CYYYHHXHHKHHXHHYYY

SEQ ID NO: 28 - protein, 18 aa, synthetic construct
  VARIANT 6: note = artificial amino acid, wherein the artificial amino acid is an oligo(alkylamino) acid of formula IIa as defined for X1
  VARIANT 12: note = artificial amino acid, wherein the artificial amino acid is an oligo(alkylamino) acid of formula IIa as defined for X1
  SITE 9: note = side chain of the lysine is linked via an amide linkage to another lysine
  VARIANT 4: note = residue may be deleted
  VARIANT 5: note = residue may be deleted
  VARIANT 7: note = residue may be deleted
  VARIANT 8: note = residue may be deleted
  VARIANT 10: note = residue may be deleted
  VARIANT 11: note = residue may be deleted
  VARIANT 13: note = residue may be deleted
  VARIANT 14: note = residue may be deleted
YYYHHXHHKHHXHHYYYC

SEQ ID NO: 29 - protein, 18 aa, synthetic construct
  VARIANT 7: note = artificial amino acid, wherein the artificial amino acid is an oligo(alkylamino) acid of formula IIa as defined for X1
  VARIANT 13: note = artificial amino acid, wherein the artificial amino acid is an oligo(alkylamino) acid of formula IIa as defined for X1
  SITE 10: note = side chain of the lysine is linked via an amide linkage to another lysine
CYYYHHXHHKHHXHHYYY

SEQ ID NO: 30 - protein, 18 aa, synthetic construct
  VARIANT 6: note = artificial amino acid, wherein the artificial amino acid is an oligo(alkylamino) acid of formula IIa as defined for X1
  VARIANT 12: note = artificial amino acid, wherein the artificial amino acid is an oligo(alkylamino) acid of formula IIa as defined for X1
  SITE 9: note = side chain of the lysine is linked via an amide linkage to another lysine
YYYHHXHHKHHXHHYYYC

SEQ ID NO: 31 - protein, 14 aa, synthetic construct
  VARIANT 6: note = artificial amino acid, wherein the artificial amino acid is an oligo(alkylamino) acid of formula IIa as defined for X1
  VARIANT 10: note = artificial amino acid, wherein the artificial amino acid is an oligo(alkylamino) acid of formula IIa as defined for X1
  SITE 8: note = side chain of the lysine is linked via an amide linkage to another lysine
CYYYHXHKHXHYYY

SEQ ID NO: 32 - protein, 14 aa, synthetic construct
  VARIANT 5: note = artificial amino acid, wherein the artificial amino acid is an oligo(alkylamino) acid of formula IIa as defined for X1
  VARIANT 9: note = artificial amino acid, wherein the artificial amino acid is an oligo(alkylamino) acid of formula IIa as defined for X1
  SITE 7: note = side chain of the lysine is linked via an amide linkage to another lysine
YYYHXHKHXHYYYC

SEQ ID NO: 33 - protein, 10 aa, synthetic construct
  VARIANT 5: note = artificial amino acid, wherein the artificial amino acid is an oligo(alkylamino) acid of formula IIa as defined for X1
  VARIANT 7: note = artificial amino acid, wherein the artificial amino acid is an oligo(alkylamino) acid of formula IIa as defined for X1
  SITE 6: note = side chain of the lysine is linked via an amide linkage to another lysine
CYYYXKXYYY

SEQ ID NO: 34 - protein, 10 aa, synthetic construct
  VARIANT 4: note = artificial amino acid, wherein the artificial amino acid is an oligo(alkylamino) acid of formula IIa as defined for X1
  VARIANT 6: note = artificial amino acid, wherein the artificial amino acid is an oligo(alkylamino) acid of formula IIa as defined for X1
  SITE 5: note = side chain of the lysine is linked via an amide linkage to another lysine
YYYXKXYYYC

SEQ ID NO: 35 - protein, 19 aa, synthetic construct
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 12: note = artificial amino acid, wherein the artificial amino acid is an oligo(alkylamino) acid of formula IIa as defined for X2
  VARIANT 14: note = artificial amino acid, wherein the artificial amino acid is an oligo(alkylamino) acid of formula IIa as defined for X2
  SITE 10: note = side chain of the lysine is linked via an amide linkage to another lysine
  VARIANT 5: note = residue may be deleted
  VARIANT 7: note = residue may be deleted
  VARIANT 9: note = residue may be deleted
  VARIANT 11: note = residue may be deleted
  VARIANT 13: note = residue may be deleted
  VARIANT 15: note = residue may be deleted
CYYYHXHXHKHXHXHYYYC

SEQ ID NO: 36 - protein, 17 aa, synthetic construct
  VARIANT 5: note = artificial amino acid, wherein the artificial amino acid is an oligo(alkylamino) acid of formula IIa as defined for X2
  VARIANT 7: note = artificial amino acid, wherein the artificial amino acid is an oligo(alkylamino) acid of formula IIa as defined for X2
  VARIANT 11: note = artificial amino acid, wherein the artificial amino acid is an oligo(alkylamino) acid of formula IIa as defined for X2
  VARIANT 13: note = artificial amino acid, wherein the artificial amino acid is an oligo(alkylamino) acid of formula IIa as defined for X2
  SITE 9: note = side chain of the lysine is linked via an amide linkage to another lysine
  VARIANT 4: note = residue may be deleted
  VARIANT 6: note = residue may be deleted
  VARIANT 8: note = residue may be deleted
  VARIANT 10: note = residue may be deleted
  VARIANT 12: note = residue may be deleted
  VARIANT 14: note = residue may be deleted
YYYHXHXHKHXHXHYYY

SEQ ID NO: 37 - protein, 19 aa, synthetic construct
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 12: note = artificial amino acid, wherein the artificial amino acid is an oligo(alkylamino) acid of formula IIa as defined for X2
  VARIANT 14: note = artificial amino acid, wherein the artificial amino acid is an oligo(alkylamino) acid of formula IIa as defined for X2
  SITE 10: note = side chain of the lysine is linked via an amide linkage to another lysine
CYYYHXHXHKHXHXHYYYC

SEQ ID NO: 38 - protein, 17 aa, synthetic construct
  VARIANT 5: note = artificial amino acid, wherein the artificial amino acid is an oligo(alkylamino) acid of formula IIa as defined for X2
  VARIANT 7: note = artificial amino acid, wherein the artificial amino acid is an oligo(alkylamino) acid of formula IIa as defined for X2
  VARIANT 11: note = artificial amino acid, wherein the artificial amino acid is an oligo(alkylamino) acid of formula IIa as defined for X2
  VARIANT 13: note = artificial amino acid, wherein the artificial amino acid is an oligo(alkylamino) acid of formula IIa as defined for X2
  SITE 9: note = side chain of the lysine is linked via an amide linkage to another lysine
YYYHXHXHKHXHXHYYY

SEQ ID NO: 39 - protein, 13 aa, synthetic construct
  VARIANT 5: note = artificial amino acid, wherein the artificial amino acid is an oligo(alkylamino) acid of formula IIa as defined for X2
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 9: note = artificial amino acid, wherein the artificial amino acid is an oligo(alkylamino) acid of formula IIa as defined for X2
  SITE 7: note = side chain of the lysine is linked via an amide linkage to another lysine
CYYYXXKXXYYYC

SEQ ID NO: 40 - protein, 11 aa, synthetic construct
  SITE 6: note = side chain of the lysine is linked via an amide linkage to another lysine
  VARIANT 4: note = artificial amino acid, wherein the artificial amino acid is an oligo(alkylamino) acid of formula IIa as defined for X2
  VARIANT 5: note = artificial amino acid, wherein the artificial amino acid is an oligo(alkylamino) acid of formula IIa as defined for X2
  VARIANT 7: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
YYYXXKXXYYY

SEQ ID NO: 41 - protein, 20 aa, synthetic construct
  VARIANT 7: note = artificial amino acid, wherein the artificial amino acid is an oligo(alkylamino) acid of formula IIa as defined for X2
  VARIANT 9: note = artificial amino acid, wherein the artificial amino acid is an oligo(alkylamino) acid of formula IIa as defined for X2
  VARIANT 13: note = artificial amino acid, wherein the artificial amino acid is an oligo(alkylamino) acid of formula IIa as defined for X2
  VARIANT 15: note = artificial amino acid, wherein the artificial amino acid is an oligo(alkylamino) acid of formula IIa as defined for X2
  SITE 11: note = side chain of the lysine is linked via an amide linkage to another lysine
  VARIANT 6: note = residue may be deleted
  VARIANT 8: note = residue may be deleted
  VARIANT 10: note = residue may be deleted
  VARIANT 12: note = residue may be deleted
  VARIANT 14: note = residue may be deleted
  VARIANT 16: note = residue may be deleted
  SITE 1: note = azido-lysine
KCYYYHXHXHKHXHXHYYYC

SEQ ID NO: 42 - protein, 18 aa, synthetic construct
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 12: note = artificial amino acid, wherein the artificial amino acid is an oligo(alkylamino) acid of formula IIa as defined for X2
  VARIANT 14: note = artificial amino acid, wherein the artificial amino acid is an oligo(alkylamino) acid of formula IIa as defined for X2
  SITE 10: note = side chain of the lysine is linked via an amide linkage to another lysine
  VARIANT 5: note = residue may be deleted
  VARIANT 7: note = residue may be deleted
  VARIANT 9: note = residue may be deleted
  VARIANT 11: note = residue may be deleted
  VARIANT 13: note = residue may be deleted
  VARIANT 15: note = residue may be deleted
  SITE 1: note = azido-lysine
KYYYHXHXHKHXHXHYYY

SEQ ID NO: 43 - protein, 20 aa, synthetic construct
  VARIANT 7: note = artificial amino acid, wherein the artificial amino acid is an oligo(alkylamino) acid of formula IIa as defined for X2
  VARIANT 9: note = artificial amino acid, wherein the artificial amino acid is an oligo(alkylamino) acid of formula IIa as defined for X2
  VARIANT 13: note = artificial amino acid, wherein the artificial amino acid is an oligo(alkylamino) acid of formula IIa as defined for X2
  VARIANT 15: note = artificial amino acid, wherein the artificial amino acid is an oligo(alkylamino) acid of formula IIa as defined for X2
  SITE 11: note = side chain of the lysine is linked via an amide linkage to another lysine
  SITE 1: note = azido-lysine
KCYYYHXHXHKHXHXHYYYC

SEQ ID NO: 44 - protein, 18 aa, synthetic construct
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 12: note = artificial amino acid, wherein the artificial amino acid is an oligo(alkylamino) acid of formula IIa as defined for X2
  VARIANT 14: note = artificial amino acid, wherein the artificial amino acid is an oligo(alkylamino) acid of formula IIa as defined for X2
  SITE 10: note = side chain of the lysine is linked via an amide linkage to another lysine
  SITE 1: note = azido-lysine
KYYYHXHXHKHXHXHYYY

SEQ ID NO: 45 - protein, 14 aa, synthetic construct
  VARIANT 6: note = artificial amino acid, wherein the artificial amino acid is an oligo(alkylamino) acid of formula IIa as defined for X2
  VARIANT 7: note = artificial amino acid, wherein the artificial amino acid is an oligo(alkylamino) acid of formula IIa as defined for X2
  VARIANT 9: note = artificial amino acid, wherein the artificial amino acid is an oligo(alkylamino) acid of formula IIa as defined for X2
  VARIANT 10: note = artificial amino acid, wherein the artificial amino acid is an oligo(alkylamino) acid of formula IIa as defined for X2
  SITE 8: note = side chain of the lysine is linked via an amide linkage to another lysine
  SITE 1: note = azido-lysine
KCYYYXXKXXYYYC

SEQ ID NO: 46 - protein, 12 aa, synthetic construct
  VARIANT 5: note = artificial amino acid, wherein the artificial amino acid is an oligo(alkylamino) acid of formula IIa as defined for X2
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 9: note = artificial amino acid, wherein the artificial amino acid is an oligo(alkylamino) acid of formula IIa as defined for X2
  SITE 7: note = side chain of the lysine is linked via an amide linkage to another lysine
  SITE 1: note = azido-lysine
KYYYXXKXXYYY

SEQ ID NO: 47 - protein, 20 aa, synthetic construct
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 12: note = artificial amino acid, wherein the artificial amino acid is an oligo(alkylamino) acid of formula IIa as defined for X2
  VARIANT 14: note = artificial amino acid, wherein the artificial amino acid is an oligo(alkylamino) acid of formula IIa as defined for X2
  SITE 10: note = side chain of the lysine is linked via an amide linkage to another lysine
  SITE 20: note = azido-lysine
  VARIANT 5: note = residue may be deleted
  VARIANT 7: note = residue may be deleted
  VARIANT 9: note = residue may be deleted
  VARIANT 11: note = residue may be deleted
  VARIANT 13: note = residue may be deleted
  VARIANT 15: note = residue may be deleted
CYYYHXHXHKHXHXHYYYCK

SEQ ID NO: 48 - protein, 18 aa, synthetic construct
  VARIANT 5: note = artificial amino acid, wherein the artificial amino acid is an oligo(alkylamino) acid of formula IIa as defined for X2
  VARIANT 7: note = artificial amino acid, wherein the artificial amino acid is an oligo(alkylamino) acid of formula IIa as defined for X2
  VARIANT 11: note = artificial amino acid, wherein the artificial amino acid is an oligo(alkylamino) acid of formula IIa as defined for X2
  VARIANT 13: note = artificial amino acid, wherein the artificial amino acid is an oligo(alkylamino) acid of formula IIa as defined for X2
  SITE 9: note = side chain of the lysine is linked via an amide linkage to another lysine
  SITE 18: note = azido-lysine
  VARIANT 4: note = residue may be deleted
  VARIANT 6: note = residue may be deleted
  VARIANT 8: note = residue may be deleted
  VARIANT 10: note = residue may be deleted
  VARIANT 12: note = residue may be deleted
  VARIANT 14: note = residue may be deleted
YYYHXHXHKHXHXHYYYK

SEQ ID NO: 49 - protein, 20 aa, synthetic construct
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 12: note = artificial amino acid, wherein the artificial amino acid is an oligo(alkylamino) acid of formula IIa as defined for X2
  VARIANT 14: note = artificial amino acid, wherein the artificial amino acid is an oligo(alkylamino) acid of formula IIa as defined for X2
  SITE 10: note = side chain of the lysine is linked via an amide linkage to another lysine
  SITE 20: note = azido-lysine
CYYYHXHXHKHXHXHYYYCK

SEQ ID NO: 50 - protein, 18 aa, synthetic construct
  VARIANT 5: note = artificial amino acid, wherein the artificial amino acid is an oligo(alkylamino) acid of formula IIa as defined for X2
  VARIANT 7: note = artificial amino acid, wherein the artificial amino acid is an oligo(alkylamino) acid of formula IIa as defined for X2
  VARIANT 11: note = artificial amino acid, wherein the artificial amino acid is an oligo(alkylamino) acid of formula IIa as defined for X2
  VARIANT 13: note = artificial amino acid, wherein the artificial amino acid is an oligo(alkylamino) acid of formula IIa as defined for X2
  SITE 9: note = side chain of the lysine is linked via an amide linkage to another lysine
  SITE 18: note = azido-lysine
YYYHXHXHKHXHXHYYYK

SEQ ID NO: 51 - protein, 14 aa, synthetic construct
  VARIANT 5: note = artificial amino acid, wherein the artificial amino acid is an oligo(alkylamino) acid of formula IIa as defined for X2
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 9: note = artificial amino acid, wherein the artificial amino acid is an oligo(alkylamino) acid of formula IIa as defined for X2
  SITE 7: note = side chain of the lysine is linked via an amide linkage to another lysine
  SITE 14: note = azido-lysine
CYYYXXKXXYYYCK

SEQ ID NO: 52 - protein, 12 aa, synthetic construct
  VARIANT 4: note = artificial amino acid, wherein the artificial amino acid is an oligo(alkylamino) acid of formula IIa as defined for X2
  VARIANT 5: note = artificial amino acid, wherein the artificial amino acid is an oligo(alkylamino) acid of formula IIa as defined for X2
  VARIANT 7: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  SITE 6: note = side chain of the lysine is linked via an amide linkage to another lysine
  SITE 12: note = azido-lysine
YYYXXKXXYYYK

SEQ ID NO: 53 - protein, 18 aa, synthetic construct
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 12: note = artificial amino acid, wherein the artificial amino acid is an oligo(alkylamino) acid of formula IIa as defined for X2
  VARIANT 14: note = artificial amino acid, wherein the artificial amino acid is an oligo(alkylamino) acid of formula IIa as defined for X2
  SITE 10: note = side chain of the lysine is linked via an amide linkage to another lysine
  VARIANT 5: note = residue may be deleted
  VARIANT 7: note = residue may be deleted
  VARIANT 9: note = residue may be deleted
  VARIANT 11: note = residue may be deleted
  VARIANT 13: note = residue may be deleted
  VARIANT 15: note = residue may be deleted
CYYYHXHXHKHXHXHYYY

SEQ ID NO: 54 - protein, 18 aa, synthetic construct
  VARIANT 5: note = artificial amino acid, wherein the artificial amino acid is an oligo(alkylamino) acid of formula IIa as defined for X2
  VARIANT 7: note = artificial amino acid, wherein the artificial amino acid is an oligo(alkylamino) acid of formula IIa as defined for X2
  VARIANT 11: note = artificial amino acid, wherein the artificial amino acid is an oligo(alkylamino) acid of formula IIa as defined for X2
  VARIANT 13: note = artificial amino acid, wherein the artificial amino acid is an oligo(alkylamino) acid of formula IIa as defined for X2
  SITE 9: note = side chain of the lysine is linked via an amide linkage to another lysine
  VARIANT 4: note = residue may be deleted
  VARIANT 6: note = residue may be deleted
  VARIANT 8: note = residue may be deleted
  VARIANT 10: note = residue may be deleted
  VARIANT 12: note = residue may be deleted
  VARIANT 14: note = residue may be deleted
YYYHXHXHKHXHXHYYYC

SEQ ID NO: 55 - protein, 18 aa, synthetic construct
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 12: note = artificial amino acid, wherein the artificial amino acid is an oligo(alkylamino) acid of formula IIa as defined for X2
  VARIANT 14: note = artificial amino acid, wherein the artificial amino acid is an oligo(alkylamino) acid of formula IIa as defined for X2
  SITE 10: note = side chain of the lysine is linked via an amide linkage to another lysine
CYYYHXHXHKHXHXHYYY

SEQ ID NO: 56 - protein, 18 aa, synthetic construct
  VARIANT 5: note = artificial amino acid, wherein the artificial amino acid is an oligo(alkylamino) acid of formula IIa as defined for X2
  VARIANT 7: note = artificial amino acid, wherein the artificial amino acid is an oligo(alkylamino) acid of formula IIa as defined for X2
  VARIANT 11: note = artificial amino acid, wherein the artificial amino acid is an oligo(alkylamino) acid of formula IIa as defined for X2
  VARIANT 13: note = artificial amino acid, wherein the artificial amino acid is an oligo(alkylamino) acid of formula IIa as defined for X2
  SITE 9: note = side chain of the lysine is linked via an amide linkage to another lysine
YYYHXHXHKHXHXHYYYC

SEQ ID NO: 57 - protein, 12 aa, synthetic construct
  VARIANT 5: note = artificial amino acid, wherein the artificial amino acid is an oligo(alkylamino) acid of formula IIa as defined for X2
  VARIANT 6: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  VARIANT 9: note = artificial amino acid, wherein the artificial amino acid is an oligo(alkylamino) acid of formula IIa as defined for X2
  SITE 7: note = side chain of the lysine is linked via an amide linkage to another lysine
CYYYXXKXXYYY

SEQ ID NO: 58 - protein, 12 aa, synthetic construct
  VARIANT 4: note = artificial amino acid, wherein the artificial amino acid is an oligo(alkylamino) acid of formula IIa as defined for X2
  VARIANT 5: note = artificial amino acid, wherein the artificial amino acid is an oligo(alkylamino) acid of formula IIa as defined for X2
  VARIANT 7: note = artificial amino acid, wherein the artificial amino acid is an oligo(alkylamino) acid of formula IIa as defined for X2
  VARIANT 8: note = artificial amino acid, wherein the artificial amino acid is an oligo(alkylamino) acid of formula IIa as defined for X2
  SITE 6: note = side chain of the lysine is linked via an amide linkage to another lysine
YYYXXKXXYYYC

SEQ ID NO: 59 - protein, 19 aa, synthetic construct
  SITE 6: note = artificial oligoamino acid succinyl-tetraethylenepentamine
  SITE 8: note = artificial oligoamino acid succinyl-tetraethylenepentamine
  SITE 12: note = artificial oligoamino acid succinyl-tetraethylenepentamine
  SITE 14: note = artificial oligoamino acid succinyl-tetraethylenepentamine
  SITE 10: note = side chain of the lysine is linked via an amide linkage to another lysine
CYYYHXHXHKHXHXHYYYC

SEQ ID NO: 60 - protein, 17 aa, synthetic construct
  SITE 5: note = artificial oligoamino acid succinyl-tetraethylenepentamine
  SITE 7: note = artificial oligoamino acid succinyl-tetraethylenepentamine
  SITE 11: note = artificial oligoamino acid succinyl-tetraethylenepentamine
  SITE 13: note = artificial oligoamino acid succinyl-tetraethylenepentamine
  SITE 9: note = side chain of the lysine is linked via an amide linkage to another lysine
YYYHXHXHKHXHXHYYY

SEQ ID NO: 61 - protein, 13 aa, synthetic construct
  SITE 6: note = artificial oligoamino acid succinyl-tetraethylenepentamine
  SITE 5: note = artificial oligoamino acid succinyl-tetraethylenepentamine
  SITE 8: note = artificial oligoamino acid succinyl-tetraethylenepentamine
  SITE 9: note = artificial oligoamino acid succinyl-tetraethylenepentamine
  SITE 7: note = side chain of the lysine is linked via an amide linkage to another lysine
CYYYXXKXXYYYC

SEQ ID NO: 62 - protein, 11 aa, synthetic construct
  SITE 5: note = artificial oligoamino acid succinyl-tetraethylenepentamine
  SITE 4: note = artificial oligoamino acid succinyl-tetraethylenepentamine
  SITE 8: note = artificial oligoamino acid succinyl-tetraethylenepentamine
  SITE 7: note = artificial oligoamino acid succinyl-tetraethylenepentamine
  SITE 6: note = side chain of the lysine is linked via an amide linkage to another lysine
YYYXXKXXYYY

SEQ ID NO: 63 - protein, 15 aa, synthetic construct
  SITE 6: note = artificial oligoamino acid succinyl-tetraethylenepentamine
  SITE 10: note = artificial oligoamino acid succinyl-tetraethylenepentamine
  SITE 8: note = side chain of the lysine is linked via an amide linkage to another lysine
CYYYHXHKHXHYYYC

SEQ ID NO: 64 - protein, 13 aa, synthetic construct
  SITE 9: note = artificial oligoamino acid succinyl-tetraethylenepentamine
  SITE 5: note = artificial oligoamino acid succinyl-tetraethylenepentamine
  SITE 7: note = side chain of the lysine is linked via an amide linkage to another lysine
YYYHXHKHXHYYY

SEQ ID NO: 65 - protein, 11 aa, synthetic construct
  SITE 5: note = artificial oligoamino acid succinyl-tetraethylenepentamine
  SITE 7: note = artificial oligoamino acid succinyl-tetraethylenepentamine
  SITE 6: note = side chain of the lysine is linked via an amide linkage to another lysine
CYYYXKXYYYC

SEQ ID NO: 66 - protein, 9 aa, synthetic construct
  SITE 4: note = artificial oligoamino acid succinyl-tetraethylenepentamine
  SITE 6: note = artificial oligoamino acid succinyl-tetraethylenepentamine
  SITE 5: note = side chain of the lysine is linked via an amide linkage to another lysine
YYYXKXYYY

SEQ ID NO: 67 - protein, 11 aa, synthetic construct
  SITE 6: note = side chain of the lysine is linked via an amide linkage to another lysine
  SITE 4: note = artificial amino acid succinyl-tetraethylenepentamine
  SITE 8: note = artificial amino acid succinyl-tetraethylenepentamine
YYHXHKHXHYY

SEQ ID NO: 68 - protein, 9 aa, synthetic construct
  SITE 3: note = artificial amino acid succinyl-tetraethylenepentamine
  SITE 7: note = artificial amino acid succinyl-tetraethylenepentamine
  SITE 5: note = side chain of the lysine is linked via an amide linkage to another lysine
YHXHKHXHY

SEQ ID NO: 69 - protein, 7 aa, synthetic construct
  SITE 4: note = side chain of the lysine is linked via an amide linkage to another lysine
  SITE 2: note = artificial amino acid succinyl-tetraethylenepentamine
  SITE 6: note = artificial amino acid succinyl-tetraethylenepentamine
HXHKHXH

SEQ ID NO: 70 - protein, 13 aa, synthetic construct
  SITE 5: note = artificial amino acid glutaryl-tetraethylenepentamine
  SITE 6: note = artificial amino acid glutaryl-tetraethylenepentamine
  SITE 8: note = artificial amino acid glutaryl-tetraethylenepentamine
  SITE 9: note = artificial amino acid glutaryl-tetraethylenepentamine
  SITE 7: note = artificial amino acid glutaryl-tetraethylenepentamine
CYYYXXKXXYYYC

SEQ ID NO: 71 - protein, 13 aa, synthetic construct
  SITE 5: note = artificial amino acid cyclohexanedicarboxyl-tetraethylenepentamine
  SITE 6: note = artificial amino acid cyclohexanedicarboxyl-tetraethylenepentamine
  SITE 8: note = artificial amino acid cyclohexanedicarboxyl-tetraethylenepentamine
  SITE 9: note = artificial amino acid cyclohexanedicarboxyl-tetraethylenepentamine
  SITE 7: note = side chain of the lysine is linked via an amide linkage to another lysine
CYYYXXKXXYYYC

SEQ ID NO: 72 - protein, 13 aa, synthetic construct
  SITE 7: note = side chain of the lysine is linked via an amide linkage to another lysine
  SITE 5: note = artificial amino acid phthalyl-tetraethylenepentamine
  SITE 6: note = artificial amino acid phthalyl-tetraethylenepentamine
  SITE 8: note = artificial amino acid phthalyl-tetraethylenepentamine
  SITE 9: note = artificial amino acid phthalyl-tetraethylenepentamine
CYYYXXKXXYYYC

SEQ ID NO: 73 - protein, 13 aa, synthetic construct
  SITE 7: note = side chain of the lysine is linked via an amide linkage to another lysine
  SITE 5: note = artificial amino acid naphthalenedicarboxyl-tetraethylenepentamine
  SITE 6: note = artificial amino acid naphthalenedicarboxyl-tetraethylenepentamine
  SITE 8: note = artificial amino acid naphthalenedicarboxyl-tetraethylenepentamine
  SITE 9: note = artificial amino acid naphthalenedicarboxyl-tetraethylenepentamine
CYYYXXKXXYYYC

SEQ ID NO: 74 - protein, 13 aa, synthetic construct
  SITE 7: note = side chain of the lysine is linked via an amide linkage to another lysine
  SITE 5: note = artificial amino acid glutaryl-triethylenetetramine
  SITE 6: note = artificial amino acid glutaryl-triethylenetetramine
  SITE 8: note = artificial amino acid glutaryl-triethylenetetramine
  SITE 9: note = artificial amino acid glutaryl-triethylenetetramine
CYYYXXKXXYYYC

SEQ ID NO: 75 - protein, 13 aa, synthetic construct
  SITE 7: note = side chain of the lysine is linked via an amide linkage to another lysine
  SITE 5: note = artificial amino acid glutaryl-N,N'-bis-(3-aminopropyl)-ethylenediamine (GEIPA)
  SITE 6: note = artificial amino acid glutaryl-N,N'-bis-(3-aminopropyl)-ethylenediamine (GEIPA)
  SITE 8: note = artificial amino acid glutaryl-N,N'-bis-(3-aminopropyl)-ethylenediamine (GEIPA)
  SITE 9: note = artificial amino acid glutaryl-N,N'-bis-(3-aminopropyl)-ethylenediamine (GEIPA)
CYYYXXKXXYYYC

SEQ ID NO: 76 - protein, 13 aa, synthetic construct
  SITE 7: note = side chain of the lysine is linked via an amide linkage to another lysine
  SITE 5: note = artificial amino acid 1,1-cyclohexanediacetyl-tetraethylenepentamine
  SITE 6: note = artificial amino acid 1,1-cyclohexanediacetyl-tetraethylenepentamine
  SITE 8: note = artificial amino acid 1,1-cyclohexanediacetyl-tetraethylenepentamine
  SITE 9: note = artificial amino acid 1,1-cyclohexanediacetyl-tetraethylenepentamine
CYYYXXKXXYYYC

SEQ ID NO: 77 - protein, 13 aa, synthetic construct
  SITE 7: note = side chain of the lysine is linked via an amide linkage to another lysine
  SITE 5: note = artificial amino acid diglycolyl-tetraethylenepentamine
  SITE 6: note = artificial amino acid diglycolyl-tetraethylenepentamine
  SITE 8: note = artificial amino acid diglycolyl-tetraethylenepentamine
  SITE 9: note = artificial amino acid diglycolyl-tetraethylenepentamine
CYYYXXKXXYYYC

SEQ ID NO: 78 - protein, 13 aa, synthetic construct
  SITE 7: note = side chain of the lysine is linked via an amide linkage to another lysine
  SITE 5: note = artificial amino acid trifluoroethyl-iminodiacetyl-tetraethylenepentamine
  SITE 6: note = artificial amino acid trifluoroethyl-iminodiacetyl-tetraethylenepentamine
  SITE 8: note = artificial amino acid trifluoroethyl-iminodiacetyl-tetraethylenepentamine
  SITE 9: note = artificial amino acid trifluoroethyl-iminodiacetyl-tetraethylenepentamine
CYYYXXKXXYYYC

SEQ ID NO: 79 - protein, 13 aa, synthetic construct
  SITE 7: note = side chain of the lysine is linked via an amide linkage to another lysine
  SITE 5: note = artificial amino acid iminodiacetyl-tetraethylenepentamine
  SITE 6: note = artificial amino acid iminodiacetyl-tetraethylenepentamine
  SITE 8: note = artificial amino acid iminodiacetyl-tetraethylenepentamine
  SITE 9: note = artificial amino acid iminodiacetyl-tetraethylenepentamine
CYYYXXKXXYYYC

SEQ ID NO: 80 - protein, 9 aa, synthetic construct
  SITE 5: note = side chain of the lysine is linked via an amide linkage to another lysine
  SITE 4: note = artificial amino acid glutaryl-tetraethylenepentamine
  SITE 6: note = artificial amino acid glutaryl-tetraethylenepentamine
YYYXKXYYY

SEQ ID NO: 81 - protein, 9 aa, synthetic construct
  SITE 5: note = side chain of the lysine is linked via an amide linkage to another lysine
  SITE 4: note = artificial amino acid cyclohexanedicarboxyl-tetraethylenepentamine
  SITE 6: note = artificial amino acid cyclohexanedicarboxyl-tetraethylenepentamine
YYYXKXYYY

SEQ ID NO: 82 - protein, 9 aa, synthetic construct
  SITE 5: note = side chain of the lysine is linked via an amide linkage to another lysine
  SITE 4: note = artificial amino acid cyclohexanedicarboxyl-tetraethylenepentamine
  SITE 6: note = artificial amino acid cyclohexanedicarboxyl-tetraethylenepentamine
YYYXKXYYY

SEQ ID NO: 83 - protein, 9 aa, synthetic construct
  SITE 5: note = side chain of the lysine is linked via an amide linkage to another lysine
  SITE 4: note = artificial amino acid naphthalenedicarboxyl-tetraethylenepentamine
  SITE 6: note = artificial amino acid naphthalenedicarboxyl-tetraethylenepentamine
YYYXKXYYY

SEQ ID NO: 84 - protein, 9 aa, synthetic construct
  SITE 5: note = side chain of the lysine is linked via an amide linkage to another lysine
  SITE 4: note = artificial amino acid glutaryl-triethylenetetramine
  SITE 6: note = artificial amino acid glutaryl-triethylenetetramine
YYYXKXYYY

SEQ ID NO: 85 - protein, 9 aa, synthetic construct
  SITE 5: note = side chain of the lysine is linked via an amide linkage to another lysine
  SITE 4: note = artificial amino acid glutaryl-N,N'-bis-(3-aminopropyl)-ethylenediamine (GEIPA)
  SITE 6: note = artificial amino acid glutaryl-N,N'-bis-(3-aminopropyl)-ethylenediamine (GEIPA)
YYYXKXYYY

SEQ ID NO: 86 - protein, 9 aa, synthetic construct
  SITE 5: note = side chain of the lysine is linked via an amide linkage to another lysine
  SITE 4: note = artificial amino acid 1,1-cyclohexanediacetyl-tetraethylenepentamine
  SITE 6: note = artificial amino acid 1,1-cyclohexanediacetyl-tetraethylenepentamine
YYYXKXYYY

SEQ ID NO: 87 - protein, 9 aa, synthetic construct
  SITE 5: note = side chain of the lysine is linked via an amide linkage to another lysine
  SITE 4: note = artificial amino acid diglycolyl-tetraethylenepentamine
  SITE 6: note = artificial amino acid diglycolyl-tetraethylenepentamine
YYYXKXYYY

SEQ ID NO: 88 - protein, 9 aa, synthetic construct
  SITE 5: note = side chain of the lysine is linked via an amide linkage to another lysine
  SITE 4: note = artificial amino acid trifluoroethyl-iminodiacetyl-tetraethylenepentamine
  SITE 6: note = artificial amino acid trifluoroethyl-iminodiacetyl-tetraethylenepentamine
YYYXKXYYY

SEQ ID NO: 89 - protein, 9 aa, synthetic construct
  SITE 5: note = side chain of the lysine is linked via an amide linkage to another lysine
  SITE 4: note = artificial amino acid iminodiacetyl-tetraethylenepentamine
  SITE 6: note = artificial amino acid iminodiacetyl-tetraethylenepentamine
YYYXKXYYY

SEQ ID NO: 90 - protein, 13 aa, synthetic construct
  SITE 7: note = side chain of the lysine is linked via an amide linkage to another lysine
  SITE 5: note = artificial amino acid glutaryl-tetraethylenepentamine (Gtp)
  SITE 9: note = artificial amino acid glutaryl-tetraethylenepentamine (Gtp)
YYYHXHKHXHYYY

SEQ ID NO: 91 - protein, 13 aa, synthetic construct
  SITE 7: note = side chain of the lysine is linked via an amide linkage to another lysine
  SITE 5: note = artificial amino acid cyclohexanedicarboxyl-tetraethylenepentamine (Htp)
  SITE 9: note = artificial amino acid cyclohexanedicarboxyl-tetraethylenepentamine (Htp)
YYYHXHKHXHYYY

SEQ ID NO: 92 - protein, 13 aa, synthetic construct
  SITE 7: note = side chain of the lysine is linked via an amide linkage to another lysine
  SITE 5: note = artificial amino acid phthalyl-tetraethylenepentamine (Ptp)
  SITE 9: note = artificial amino acid phthalyl-tetraethylenepentamine (Ptp)
YYYHXHKHXHYYY

SEQ ID NO: 93 - protein, 13 aa, synthetic construct
  SITE 7: note = side chain of the lysine is linked via an amide linkage to another lysine
  SITE 5: note = artificial amino acid naphthalenedicarboxyl-tetraethylenepentamine (Ntp)
  SITE 9: note = artificial amino acid naphthalenedicarboxyl-tetraethylenepentamine (Ntp)
YYYHXHKHXHYYY

SEQ ID NO: 94 - protein, 13 aa, synthetic construct
  SITE 7: note = side chain of the lysine is linked via an amide linkage to another lysine
  SITE 5: note = artificial amino acid glutaryl-triethylenetetramine (Gtt)
  SITE 9: note = artificial amino acid glutaryl-triethylenetetramine (Gtt)
YYYHXHKHXHYYY

SEQ ID NO: 95 - protein, 13 aa, synthetic construct
  SITE 7: note = side chain of the lysine is linked via an amide linkage to another lysine
  SITE 5: note = artificial amino acid glutaryl-N,N'-bis-(3-aminopropyl)-ethylenediamine (GEIPA)
  SITE 9: note = artificial amino acid glutaryl-N,N'-bis-(3-aminopropyl)-ethylenediamine (GEIPA)
YYYHXHKHXHYYY

SEQ ID NO: 96 - protein, 13 aa, synthetic construct
  SITE 7: note = side chain of the lysine is linked via an amide linkage to another lysine
  SITE 5: note = artificial amino acid 1,1-cyclohexanediacetyl-tetraethylenepentamine (chGtp)
  SITE 9: note = artificial amino acid 1,1-cyclohexanediacetyl-tetraethylenepentamine (chGtp)
YYYHXHKHXHYYY

SEQ ID NO: 97 - protein, 13 aa, synthetic construct
  SITE 7: note = side chain of the lysine is linked via an amide linkage to another lysine
  SITE 5: note = artificial amino acid diglycolyl-tetraethylenepentamine (dGtp)
  SITE 9: note = artificial amino acid diglycolyl-tetraethylenepentamine (dGtp)
YYYHXHKHXHYYY

SEQ ID NO: 98 - protein, 13 aa, synthetic construct
  SITE 7: note = side chain of the lysine is linked via an amide linkage to another lysine
  SITE 5: note = artificial amino acid trifluoroethyl-iminodiacetyl-tetraethylenepentamine (TFE-IDAtp)
  SITE 9: note = artificial amino acid trifluoroethyl-iminodiacetyl-tetraethylenepentamine (TFE-IDAtp)
YYYHXHKHXHYYY

SEQ ID NO: 99 - protein, 13 aa, synthetic construct
  SITE 7: note = side chain of the lysine is linked via an amide linkage to another lysine
  SITE 5: note = artificial amino acid iminodiacetyl-tetraethylenepentamine (IDAtp)
  SITE 9: note = artificial amino acid iminodiacetyl-tetraethylenepentamine (IDAtp)
YYYHXHKHXHYYY

SEQ ID NO: 100 - protein, 16 aa, synthetic construct
  SITE 1: note = azido-lysine
  SITE 9: note = side chain of the lysine is linked via an amide linkage to another lysine
  SITE 7: note = artificial amino acid succinyl-tetraethylenepentamine (Stp)
  SITE 11: note = artificial amino acid succinyl-tetraethylenepentamine (Stp)
KCYYYHXHKHXHYYYC

SEQ ID NO: 101 - protein, 14 aa, synthetic construct
  SITE 1: note = azido-lysine
  SITE 6: note = artificial amino acid succinyl-tetraethylenepentamine (Stp)
  SITE 10: note = artificial amino acid succinyl-tetraethylenepentamine (Stp)
  SITE 8: note = side chain of the lysine is linked via an amide linkage to another lysine
KYYYHXHKHXHYYY

SEQ ID NO: 102 - protein, 12 aa, synthetic construct
  SITE 1: note = azido-lysine
  SITE 7: note = side chain of the lysine is linked via an amide linkage to another lysine
  SITE 6: note = artificial amino acid succinyl-tetraethylenepentamine (Stp)
  SITE 8: note = artificial amino acid succinyl-tetraethylenepentamine (Stp)
KCYYYXKXYYYC

SEQ ID NO: 103 - protein, 10 aa, synthetic construct
  SITE 1: note = azido-lysine
  SITE 5: note = artificial amino acid succinyl-tetraethylenepentamine (Stp)
  SITE 7: note = artificial amino acid succinyl-tetraethylenepentamine (Stp)
  SITE 6: note = side chain of the lysine is linked via an amide linkage to another lysine
KYYYXKXYYY

SEQ ID NO: 104 - protein, 20 aa, synthetic construct
  SITE 1: note = azido-lysine
  SITE 11: note = side chain of the lysine is linked via an amide linkage to another lysine
  SITE 7: note = artificial amino acid succinyl-tetraethylenepentamine (Stp)
  SITE 9: note = artificial amino acid succinyl-tetraethylenepentamine (Stp)
  SITE 13: note = artificial amino acid succinyl-tetraethylenepentamine (Stp)
  SITE 15: note = artificial amino acid succinyl-tetraethylenepentamine (Stp)
KCYYYHXHXHKHXHXHYYYC

SEQ ID NO: 105 - protein, 18 aa, synthetic construct
  SITE 1: note = azido-lysine
  SITE 6: note = artificial amino acid succinyl-tetraethylenepentamine (Stp)
  SITE 8: note = artificial amino acid succinyl-tetraethylenepentamine (Stp)
  SITE 12: note = artificial amino acid succinyl-tetraethylenepentamine (Stp)
  SITE 14: note = artificial amino acid succinyl-tetraethylenepentamine (Stp)
  SITE 10: note = side chain of the lysine is linked via an amide linkage to another lysine
KYYYHXHXHKHXHXHYYY

SEQ ID NO: 106 - protein, 14 aa, synthetic construct
  SITE 1: note = azido-lysine
  SITE 8: note = side chain of the lysine is linked via an amide linkage to another lysine
  SITE 6: note = artificial amino acid succinyl-tetraethylenepentamine (Stp)
  SITE 7: note = artificial amino acid succinyl-tetraethylenepentamine (Stp)
  SITE 9: note = artificial amino acid succinyl-tetraethylenepentamine (Stp)
  SITE 10: note = artificial amino acid succinyl-tetraethylenepentamine (Stp)
KCYYYXXKXXYYYC

SEQ ID NO: 107 - protein, 12 aa, synthetic construct
  SITE 1: note = azido-lysine
  SITE 5: note = artificial amino acid succinyl-tetraethylenepentamine (Stp)
  SITE 6: note = artificial amino acid succinyl-tetraethylenepentamine (Stp)
  SITE 8: note = artificial amino acid succinyl-tetraethylenepentamine (Stp)
  SITE 9: note = artificial amino acid succinyl-tetraethylenepentamine (Stp)
  SITE 7: note = side chain of the lysine is linked via an amide linkage to another lysine
KYYYXXKXXYYY

SEQ ID NO: 108 - RNA, 100 nt, synthetic construct
  source 1..100: mol_type = other RNA
  modified_base 1: mod_base = gm; note = phosphorothioated RNA base
  modified_base 2: mod_base = OTHER; note = phosphorothioated 2'-O-methyladenosine
  modified_base 3: mod_base = cm; note = phosphorothioated RNA base
  modified_base 97: mod_base = um; note = phosphorothioated RNA base
  modified_base 98: mod_base = um; note = phosphorothioated RNA base
  modified_base 99: mod_base = um; note = phosphorothioated RNA base
gaccaggatgggcaccacccgttttagagctagaaatagcaagttaaaataaggctagtccgttatcaacttgaaaaagtggcaccgagtcggtgctttt

SEQ ID NO: 109 - RNA, 100 nt, synthetic construct
  source 1..100: mol_type = other RNA
  modified_base 1: mod_base = gm; note = phosphorothioated RNA base
  modified_base 2: mod_base = cm; note = phosphorothioated RNA base
  modified_base 3: mod_base = um; note = phosphorothioated RNA base
  modified_base 97: mod_base = um; note = phosphorothioated RNA base
  modified_base 98: mod_base = um; note = phosphorothioated RNA base
  modified_base 99: mod_base = um; note = phosphorothioated RNA base
gctgaagcactgcacgccgtgttttagagctagaaatagcaagttaaaataaggctagtccgttatcaacttgaaaaagtggcaccgagtcggtgctttt

SEQ ID NO: 110 - DNA, 127 bp, synthetic construct
  source 1..127: mol_type = other DNA
  modified_base 1: mod_base = OTHER; note = phosphorothioated DNA base
  modified_base 2: mod_base = OTHER; note = phosphorothioated DNA base
  modified_base 125: mod_base = OTHER; note = phosphorothioated DNA base
  modified_base 126: mod_base = OTHER; note = phosphorothioated DNA base
gccacctacggcaagctgaccctgaagttcatctgcaccaccggcaagctgcccgtgccctggcccaccctcgtgaccaccctgagccacggcgtgcagtgcttcagccgctaccccgaccacatga

SEQ ID NO: 111 - protein, 14 aa, synthetic construct
  SITE 6: note = artificial amino acid glutaryl-tetraethylenepentamine
  SITE 7: note = artificial amino acid glutaryl-tetraethylenepentamine
  SITE 9: note = artificial amino acid glutaryl-tetraethylenepentamine
  SITE 10: note = artificial amino acid glutaryl-tetraethylenepentamine
  SITE 8: note = artificial amino acid glutaryl-tetraethylenepentamine
  SITE 1: note = azido-lysine
KCYYYXXKXXYYYC

SEQ ID NO: 112 - protein, 14 aa, synthetic construct
  SITE 6: note = artificial amino acid cyclohexanedicarboxyl-tetraethylenepentamine
  SITE 7: note = artificial amino acid cyclohexanedicarboxyl-tetraethylenepentamine
  SITE 9: note = artificial amino acid cyclohexanedicarboxyl-tetraethylenepentamine
  SITE 10: note = artificial amino acid cyclohexanedicarboxyl-tetraethylenepentamine
  SITE 8: note = side chain of the lysine is linked via an amide linkage to another lysine
  SITE 1: note = azido-lysine
KCYYYXXKXXYYYC

SEQ ID NO: 113 - protein, 14 aa, synthetic construct
  SITE 8: note = side chain of the lysine is linked via an amide linkage to another lysine
  SITE 6: note = artificial amino acid phthalyl-tetraethylenepentamine
  SITE 7: note = artificial amino acid phthalyl-tetraethylenepentamine
  SITE 9: note = artificial amino acid phthalyl-tetraethylenepentamine
  SITE 10: note = artificial amino acid phthalyl-tetraethylenepentamine
  SITE 1: note = azido-lysine
KCYYYXXKXXYYYC

SEQ ID NO: 114 - protein, 14 aa, synthetic construct
  SITE 8: note = side chain of the lysine is linked via an amide linkage to another lysine
  SITE 6: note = artificial amino acid naphthalenedicarboxyl-tetraethylenepentamine
  SITE 7: note = artificial amino acid naphthalenedicarboxyl-tetraethylenepentamine
  SITE 9: note = artificial amino acid naphthalenedicarboxyl-tetraethylenepentamine
  SITE 10: note = artificial amino acid naphthalenedicarboxyl-tetraethylenepentamine
  SITE 1: note = azido-lysine
KCYYYXXKXXYYYC

SEQ ID NO: 115 - protein, 14 aa, synthetic construct
  SITE 8: note = side chain of the lysine is linked via an amide linkage to another lysine
  SITE 7: note = artificial amino acid glutaryl-triethylenetetramine
  SITE 6: note = artificial amino acid glutaryl-triethylenetetramine
  SITE 10: note = artificial amino acid glutaryl-triethylenetetramine
  SITE 9: note = artificial amino acid glutaryl-triethylenetetramine
  SITE 1: note = azido-lysine
KCYYYXXKXXYYYC

SEQ ID NO: 116 - protein, 14 aa, synthetic construct
  SITE 8: note = side chain of the lysine is linked via an amide linkage to another lysine
  SITE 7: note = artificial amino acid glutaryl-N,N'-bis-(3-aminopropyl)-ethylenediamine (GEIPA)
  SITE 6: note = artificial amino acid glutaryl-N,N'-bis-(3-aminopropyl)-ethylenediamine (GEIPA)
  SITE 10: note = artificial amino acid glutaryl-N,N'-bis-(3-aminopropyl)-ethylenediamine (GEIPA)
  SITE 9: note = artificial amino acid glutaryl-N,N'-bis-(3-aminopropyl)-ethylenediamine (GEIPA)
  SITE 1: note = azido-lysine
KCYYYXXKXXYYYC

SEQ ID NO: 117 - protein, 14 aa, synthetic construct
  SITE 8: note = side chain of the lysine is linked via an amide linkage to another lysine
  SITE 7: note = artificial amino acid 1,1-cyclohexanediacetyl-tetraethylenepentamine
  SITE 6: note = artificial amino acid 1,1-cyclohexanediacetyl-tetraethylenepentamine
  SITE 10: note = artificial amino acid 1,1-cyclohexanediacetyl-tetraethylenepentamine
  SITE 9: note = artificial amino acid 1,1-cyclohexanediacetyl-tetraethylenepentamine
  SITE 1: note = azido-lysine
KCYYYXXKXXYYYC

SEQ ID NO: 118 - protein, 14 aa, synthetic construct
  SITE 8: note = side chain of the lysine is linked via an amide linkage to another lysine
  SITE 7: note = artificial amino acid diglycolyl-tetraethylenepentamine
  SITE 6: note = artificial amino acid diglycolyl-tetraethylenepentamine
  SITE 10: note = artificial amino acid diglycolyl-tetraethylenepentamine
  SITE 9: note = artificial amino acid diglycolyl-tetraethylenepentamine
  SITE 1: note = azido-lysine
KCYYYXXKXXYYYC

SEQ ID NO: 119 - protein, 14 aa, synthetic construct
  SITE 8: note = side chain of the lysine is linked via an amide linkage to another lysine
  SITE 7: note = artificial amino acid trifluoroethyl-iminodiacetyl-tetraethylenepentamine
  SITE 6: note = artificial amino acid trifluoroethyl-iminodiacetyl-tetraethylenepentamine
  SITE 10: note = artificial amino acid trifluoroethyl-iminodiacetyl-tetraethylenepentamine
  SITE 9: note = artificial amino acid trifluoroethyl-iminodiacetyl-tetraethylenepentamine
  SITE 1: note = azido-lysine
KCYYYXXKXXYYYC

SEQ ID NO: 120 - protein, 14 aa, synthetic construct
  SITE 8: note = side chain of the lysine is linked via an amide linkage to another lysine
  SITE 7: note = artificial amino acid iminodiacetyl-tetraethylenepentamine
  SITE 6: note = artificial amino acid iminodiacetyl-tetraethylenepentamine
  SITE 10: note = artificial amino acid iminodiacetyl-tetraethylenepentamine
  SITE 9: note = artificial amino acid iminodiacetyl-tetraethylenepentamine
  SITE 1: note = azido-lysine
KCYYYXXKXXYYYC

SEQ ID NO: 121 - protein, 10 aa, synthetic construct
  SITE 6: note = side chain of the lysine is linked via an amide linkage to another lysine
  SITE 5: note = artificial amino acid glutaryl-tetraethylenepentamine
  SITE 7: note = artificial amino acid glutaryl-tetraethylenepentamine
  SITE 1: note = azido-lysine
KYYYXKXYYY

SEQ ID NO: 122 - protein, 10 aa, synthetic construct
  SITE 6: note = side chain of the lysine is linked via an amide linkage to another lysine
  SITE 5: note = artificial amino acid cyclohexanedicarboxyl-tetraethylenepentamine
  SITE 7: note = artificial amino acid cyclohexanedicarboxyl-tetraethylenepentamine
  SITE 1: note = azido-lysine
KYYYXKXYYY

SEQ ID NO: 123 - protein, 10 aa, synthetic construct
  SITE 6: note = side chain of the lysine is linked via an amide linkage to another lysine
  SITE 5: note = artificial amino acid cyclohexanedicarboxyl-tetraethylenepentamine
  SITE 7: note = artificial amino acid cyclohexanedicarboxyl-tetraethylenepentamine
  SITE 1: note = azido-lysine
KYYYXKXYYY

SEQ ID NO: 124 - protein, 10 aa, synthetic construct
  SITE 6: note = side chain of the lysine is linked via an amide linkage to another lysine
  SITE 5: note = artificial amino acid naphthalenedicarboxyl-tetraethylenepentamine
  SITE 7: note = artificial amino acid naphthalenedicarboxyl-tetraethylenepentamine
  SITE 1: note = azido-lysine
KYYYXKXYYY

SEQ ID NO: 125 - protein, 10 aa, synthetic construct
  SITE 6: note = side chain of the lysine is linked via an amide linkage to another lysine
  SITE 5: note = artificial amino acid glutaryl-triethylenetetramine
  SITE 7: note = artificial amino acid glutaryl-triethylenetetramine
  SITE 1: note = azido-lysine
KYYYXKXYYY

SEQ ID NO: 126 - protein, 10 aa, synthetic construct
  SITE 6: note = side chain of the lysine is linked via an amide linkage to another lysine
  SITE 5: note = artificial amino acid glutaryl-N,N'-bis-(3-aminopropyl)-ethylenediamine (GEIPA)
  SITE 7: note = artificial amino acid glutaryl-N,N'-bis-(3-aminopropyl)-ethylenediamine (GEIPA)
  SITE 1: note = azido-lysine
KYYYXKXYYY

SEQ ID NO: 127 - protein, 10 aa, synthetic construct
  SITE 6: note = side chain of the lysine is linked via an amide linkage to another lysine
  SITE 5: note = artificial amino acid 1,1-cyclohexanediacetyl-tetraethylenepentamine
  SITE 7: note = artificial amino acid 1,1-cyclohexanediacetyl-tetraethylenepentamine
  SITE 1: note = azido-lysine
KYYYXKXYYY

SEQ ID NO: 128 - protein, 10 aa, synthetic construct
  SITE 6: note = side chain of the lysine is linked via an amide linkage to another lysine
  SITE 5: note = artificial amino acid diglycolyl-tetraethylenepentamine
  SITE 7: note = artificial amino acid diglycolyl-tetraethylenepentamine
  SITE 1: note = azido-lysine
KYYYXKXYYY

SEQ ID NO: 129 - protein, 10 aa, synthetic construct
  SITE 6: note = side chain of the lysine is linked via an amide linkage to another lysine
  SITE 5: note = artificial amino acid trifluoroethyl-iminodiacetyl-tetraethylenepentamine
  SITE 7: note = artificial amino acid trifluoroethyl-iminodiacetyl-tetraethylenepentamine
  SITE 1: note = azido-lysine
KYYYXKXYYY

SEQ ID NO: 130 - protein, 10 aa, synthetic construct
  SITE 6: note = side chain of the lysine is linked via an amide linkage to another lysine
  SITE 5: note = artificial amino acid iminodiacetyl-tetraethylenepentamine
  SITE 7: note = artificial amino acid iminodiacetyl-tetraethylenepentamine
  SITE 1: note = azido-lysine
KYYYXKXYYY

SEQ ID NO: 131 - protein, 14 aa, synthetic construct
  SITE 8: note = side chain of the lysine is linked via an amide linkage to another lysine
  SITE 6: note = artificial amino acid glutaryl-tetraethylenepentamine (Gtp)
  SITE 10: note = artificial amino acid glutaryl-tetraethylenepentamine (Gtp)
  SITE 1: note = azido-lysine
KYYYHXHKHXHYYY

SEQ ID NO: 132 - protein, 14 aa, synthetic construct
  SITE 8: note = side chain of the lysine is linked via an amide linkage to another lysine
  SITE 6: note = artificial amino acid cyclohexanedicarboxyl-tetraethylenepentamine (Htp)
  SITE 10: note = artificial amino acid cyclohexanedicarboxyl-tetraethylenepentamine (Htp)
  SITE 1: note = azido-lysine
KYYYHXHKHXHYYY

SEQ ID NO: 133 - protein, 14 aa, synthetic construct
  SITE 8: note = side chain of the lysine is linked via an amide linkage to another lysine
  SITE 6: note = artificial amino acid phthalyl-tetraethylenepentamine (Ptp)
  SITE 10: note = artificial amino acid phthalyl-tetraethylenepentamine (Ptp)
  SITE 1: note = azido-lysine
KYYYHXHKHXHYYY

SEQ ID NO: 134 - protein, 14 aa, synthetic construct
  SITE 8: note = side chain of the lysine is linked via an amide linkage to another lysine
  SITE 6: note = artificial amino acid naphthalenedicarboxyl-tetraethylenepentamine (Ntp)
  SITE 10: note = artificial amino acid naphthalenedicarboxyl-tetraethylenepentamine (Ntp)
  SITE 1: note = azido-lysine
KYYYHXHKHXHYYY

SEQ ID NO: 135 - protein, 14 aa, synthetic construct
  SITE 8: note = side chain of the lysine is linked via an amide linkage to another lysine
  SITE 6: note = artificial amino acid glutaryl-triethylenetetramine (Gtt)
  SITE 10: note = artificial amino acid glutaryl-triethylenetetramine (Gtt)
  SITE 1: note = azido-lysine
KYYYHXHKHXHYYY

SEQ ID NO: 136 - protein, 14 aa, synthetic construct
  SITE 8: note = side chain of the lysine is linked via an amide linkage to another lysine
  SITE 6: note = artificial amino acid glutaryl-N,N'-bis-(3-aminopropyl)-ethylenediamine (GEIPA)
  SITE 10: note = artificial amino acid glutaryl-N,N'-bis-(3-aminopropyl)-ethylenediamine (GEIPA)
  SITE 1: note = azido-lysine
KYYYHXHKHXHYYY

SEQ ID NO: 137 - protein, 14 aa, synthetic construct
  SITE 8: note = side chain of the lysine is linked via an amide linkage to another lysine
  SITE 6: note = artificial amino acid 1,1-cyclohexanediacetyl-tetraethylenepentamine (chGtp)
  SITE 10: note = artificial amino acid 1,1-cyclohexanediacetyl-tetraethylenepentamine (chGtp)
  SITE 1: note = azido-lysine
KYYYHXHKHXHYYY

SEQ ID NO: 138 - protein, 14 aa, synthetic construct
  SITE 8: note = side chain of the lysine is linked via an amide linkage to another lysine
  SITE 6: note = artificial amino acid diglycolyl-tetraethylenepentamine (dGtp)
  SITE 10: note = artificial amino acid diglycolyl-tetraethylenepentamine (dGtp)
  SITE 1: note = azido-lysine
KYYYHXHKHXHYYY

SEQ ID NO: 139 - protein, 14 aa, synthetic construct
  SITE 8: note = side chain of the lysine is linked via an amide linkage to another lysine
  SITE 6: note = artificial amino acid trifluoroethyl-iminodiacetyl-tetraethylenepentamine (TFE-IDAtp)
  SITE 10: note = artificial amino acid trifluoroethyl-iminodiacetyl-tetraethylenepentamine (TFE-IDAtp)
  SITE 1: note = azido-lysine
KYYYHXHKHXHYYY

SEQ ID NO: 140 - protein, 14 aa, synthetic construct
  SITE 8: note = side chain of the lysine is linked via an amide linkage to another lysine
  SITE 6: note = artificial amino acid iminodiacetyl-tetraethylenepentamine (IDAtp)
  SITE 10: note = artificial amino acid iminodiacetyl-tetraethylenepentamine (IDAtp)
  SITE 1: note = azido-lysine
KYYYHXHKHXHYYY

SEQ ID NO: 141 - protein, 12 aa, synthetic construct
  SITE 7: note = side chain of the lysine is linked via an amide linkage to another lysine
  SITE 5: note = artificial amino acid succinyl-tetraethylenepentamine
  SITE 9: note = artificial amino acid succinyl-tetraethylenepentamine
  SITE 1: note = azido-lysine
KYYHXHKHXHYY

SEQ ID NO: 142 - protein, 10 aa, synthetic construct
  SITE 4: note = artificial amino acid succinyl-tetraethylenepentamine
  SITE 8: note = artificial amino acid succinyl-tetraethylenepentamine
  SITE 6: note = side chain of the lysine is linked via an amide linkage to another lysine
  SITE 1: note = azido-lysine
KYHXHKHXHY

SEQ ID NO: 143 - protein, 8 aa, synthetic construct
  SITE 5: note = side chain of the lysine is linked via an amide linkage to another lysine
  SITE 3: note = artificial amino acid succinyl-tetraethylenepentamine
  SITE 7: note = artificial amino acid succinyl-tetraethylenepentamine
  SITE 1: note = azido-lysine
KHXHKHXH

SEQ ID NO: 144 - DNA, 21 bp, synthetic construct
  source 1..21: mol_type = other DNA
  misc_feature 1..19: note = RNA
  misc_feature 20..21: note = DNA
  modified_base 7: mod_base = um
  modified_base 4: mod_base = um
  modified_base 2: mod_base = um
  modified_base 5: mod_base = cm
  modified_base 10: mod_base = cm
  modified_base 11: mod_base = cm
  modified_base 14: mod_base = cm
  modified_base 18: mod_base = cm
  modified_base 21: mod_base = OTHER; note = phosphorothioated DNA base
atatcatggccgacaagcatt

SEQ ID NO: 145 - DNA, 21 bp, synthetic construct
  modified_base 21: mod_base = OTHER; note = phosphorothioated DNA base
  source 1..21: mol_type = other DNA
  misc_feature 1..19: note = RNA
  misc_feature 20..21: note = DNA
  modified_base 12: mod_base = cm
  modified_base 17: mod_base = um
tgcttgtcggccatgatattt

SEQ ID NO: 146 - DNA, 21 bp, synthetic construct
  source 1..21: mol_type = other DNA
  misc_feature 1..19: note = RNA
  misc_feature 20..21: note = DNA
  modified_base 2: mod_base = um
  modified_base 4: mod_base = um
  modified_base 6..7: mod_base = um
  modified_base 10..11: mod_base = cm
  modified_base 12: mod_base = um
  modified_base 14: mod_base = um
  modified_base 16..17: mod_base = um
  modified_base 21: mod_base = OTHER; note = phosphorothioated DNA base
atgtattggcctgtattagtt

SEQ ID NO: 147 - DNA, 21 bp, synthetic construct
  source 1..21: mol_type = other DNA
  misc_feature 1..19: note = RNA
  misc_feature 20..21: note = DNA
  modified_base 2: mod_base = um
  modified_base 5: mod_base = um
  modified_base 7: mod_base = cm
  modified_base 12: mod_base = cm
  modified_base 15: mod_base = um
  modified_base 17: mod_base = cm
  modified_base 21: mod_base = OTHER; note = phosphorothioated DNA base
ctaatacaggccaatacattt

(19) United States
(12) Patent Application Publication
(10) Pub. No.: US 2026/0295075 A1
(43) Pub. Date: Oct. 1, 2026

(54) ARTIFICIAL AMINO ACID CONTAINING LIPO-OLIGOMERS FOR RIBONUCLEOPROTEIN DELIVERY

(71) Applicant: LUDWIG-MAXIMILIANS-UNIVERSITÄT MÜNCHEN, München (DE)

(72) Inventors: Ernst WAGNER, München (DE); Ulrich LÄCHELT, München (DE); Yi LIN, München (DE); Mina YAZDI, München (DE); Anna-Lina LESSL, München (DE)

(21) Appl. No.: 19/127,576

(22) PCT Filed: Nov. 6, 2023
(86) PCT No.: PCT/EP2023/080853

§ 371 Date: May 6, 2025

(30) Foreign Application Priority Data
Nov. 7, 2022 (EP) 22205883.6
Nov. 22, 2022 (EP) 22208928.6

(51) Int. Cl. A61K 48/00; A61K 47/54; A61K 47/64; A61K 47/69; C12N 9/22; C12N 15/11; C12N 15/113; C12N 15/88; C12N 15/90

(52) U.S. Cl. CPC A61K 48/0033; A61K 47/543; A61K 47/64; A61K 47/6929; C12N 9/226; C12N 15/11; C12N 15/113; C12N 15/88; C12N 15/907; C12N 2310/14; C12N 2310/20; C12N 2310/3233

(57) ABSTRACT

The invention relates to nanoparticles for Cas protein/gRNA ribonucleoprotein (RNP) complex, siRNA or PMO delivery comprising one or more Cas protein/gRNA RNP complex(es), siRNA(s) or PMO(s) as cargo; and a carrier comprising a sequence-defined T-shape lipo-oligomer comprising a tyrosine tripeptide and either a single artificial amino acid or two hydrophobic artificial amino acids at either side of a central lysine branching point and a hydrophobic tail comprising two fatty acids, wherein the artificial amino acid is an oligo(alkylamino) acid. The invention further relates to therapeutic and non-therapeutic uses thereof and to an in vitro method for transfecting mammalian cells.

### CROSS REFERENCE TO RELATED APPLICATIONS

This application is a national stage application under 35 U.S.C. 371 and claims the benefit of PCT Application No. PCT/EP2023/080853 having an international filing date of Nov. 6, 2023, which designated the United States, which PCT application claimed the benefit of European Patent Application No. 22205883.6 filed 7 Nov. 2022, and European Patent Application No. 22208928.6 filed 22 Nov. 2022, the disclosures of each of which are incorporated herein by reference in their entireties.

### REFERENCE TO SEQUENCE LISTING

This application contains a Sequence Listing submitted as an electronic ST.26 XML file named 118371P1272PCUS_sequence_listing.xml, having a size of 458,752 bytes, and created on Apr. 28, 2026. The information contained in this XML file is hereby incorporated by reference in its entirety.

### FIELD OF THE INVENTION

The invention relates to nanoparticles for Cas protein/gRNA ribonucleoprotein (RNP) complex, SIRNA or PMO delivery comprising one or more Cas protein/gRNA RNP complex(es), siRNA(s) or PMO(s) as cargo; and a carrier comprising a sequence-defined T-shape lipo-oligomer comprising a tyrosine tripeptide and either a single artificial amino acid or two hydrophobic artificial amino acids at either side of a central lysine branching point and a hydrophobic tail comprising two fatty acids, wherein the artificial amino acid is an oligo(alkylamino) acid. The invention further relates to therapeutic and non-therapeutic uses thereof and to an in vitro method for transfecting mammalian cells.

### BACKGROUND

Biopharmaceuticals, such as therapeutic proteins and therapeutic nucleic acids have become increasingly important. Also, the Clustered Regularly Interspaced Short Palindromic Repeat (CRISPR) system, an adaptable DNA cleavage system found in bacteria, has evolved as a groundbreaking genetic engineering tool. It allows genome editing in both single cells and whole organisms with high precision by introducing double-strand breaks (DSB) at targeted genomic loci and offers immense therapeutic opportunities. It has been utilized as an efficient RNA-guided genome-editing tool in numerous species, including human cells, and has become the most popular genome engineering approach. The CRISPR/Cas system has been used for biomedical applications such as transcriptional control, epigenetic modifications, genome-wide screening and chromosomal imaging, and treatment of genetic disorders. Engineered CRISPR systems contain at least two components, a guide RNA (gRNA) and a CRISPR-associated endonuclease (Cas protein).

The target sequence of the programmable endonuclease Cas is controlled by a guide RNA (a combination of crRNA and tracrRNA) or a single guide RNA (sgRNA). Endogenous cellular mechanisms are exploited for repair of double strand breaks, non-homologous end joining (NHEJ) results in mutations and gene knockout, while homology directed repair (HDR) can result in precise gene editing and DNA sequence knock-in when using donor-DNA (template).

For efficient genome editing, a successful intracellular delivery of the CRISPR/Cas9 components is essential and represents one of the major challenges for CRISPR/Cas-based genome editing. So far, the most common strategy is based on the delivery of the CRISPR/Cas encoding DNA sequences, such as plasmid DNA encoding the Cas protein and a guide RNA or single guide RNA or in vitro transcribed RNA molecules.

However, delivery of pre-assembled ribonucleoproteins (RNPs) comprising the Cas protein and the guide RNA (gRNA) is generally considered as the most straightforward and efficient strategy. The direct delivery of the Cas protein complexed with gRNA or sgRNA has several advantages over the delivery of the corresponding nucleic acid precursors, as the ribonucleoprotein (RNP) complex is immediately functional without the requirement of transcription and translation. Furthermore, there is no risk of spontaneous genome integration, and timely degradation reduces potential off-target effects.

Yet, direct delivery of the Cas/gRNA complex remains to be a challenge as it requires suitable carrier systems, due to poor membrane permeability. Also, nucleic acids and proteins are susceptible to enzymatic degradation and incorporation into a carrier system potentially increases their stability in vivo. Different non-viral delivery technologies have evolved for the direct delivery of the RNP complexes (Wan, T et al., Material solutions for delivery of CRISPR/Cas-based genome editing tools:

Current status and future outlook, Materials Today, 2019, 26:40-66), including cell-penetrating peptides, DNA nanoclews, gold nanoparticles, polymeric systems, black phosphorus nanosheets, hydrogels, or lipid nanoparticles. Nonetheless, the need for better carriers for stable RNP packaging, high cellular uptake, efficient endosomal escape, and nuclear entry while preserving biological activity remains.

Sequence-defined cationic oligomers containing artificial amino acids provide delivery systems with high chemical precision and flexibility. Artificial amino acids, such as oligo(ethylamino) acids containing the PEI-like aminoethylene motif, are assembled together with natural amino acids on solid-phase to sequence-defined oligomers.

Sequence-defined oligomers, such as oligo(ethylamino) amides, based on artificial oligoamino acids and solid-phase synthesis have recently been developed as a platform for the delivery of nucleic acids (Schaffert, D. et al., Solid-phase synthesis of sequence-defined T-, i-, and U-shape polymers for pDNA and siRNA Delivery, Angew. Chem., Int. Ed. 2011, 50 (38), 8986-8989), proteins (Zhang, P. et al., Enhanced Intracellular Protein Transduction by Sequence Defined Tetra-Oleoyl Oligoaminoamides Targeted for Cancer Therapy, Adv. Funct. Mater., 2015, 25, 6627-6636), and drugs (Truebenbach, I. et al., Combination Chemotherapy of L1210 Tumors in Mice with Pretubulysin and Methotrexate Lipo-Oligomer Nanoparticles, Mol. Pharmaceutics, 2019, 16, 2405-2417). They combine the advantages of aminoethylene-based polymers with the chemical precision of peptides and enable cargo-specific optimization. These sequence-defined oligomers may further contain a lipid portion (lipo-oligomer).

Particularly for nucleic acids, a convenient and frequently used mechanism is based on the ionic interaction between the negatively charged nucleic acid and polycations. The nucleic acid condensation by positively charged polymers is an entropy driven process and produces nanosized complexes, also referred to as polyplexes. Stable packaging predominantly depends on the size and charge density of the cationic polymer. The nucleic acid complexation by cationic polymers produces particles with positive surface potential, which can induce internalization by electrostatic interaction with the negatively charged cell membrane. This may be further improved by attachment of targeting ligands. Following uptake, the entrapped polyplexes have to be released from the vesicles for nuclear delivery. Endosomal escape seems to be enhanced by specific protonation of ionizable groups in the endosomal pH range, which is, e.g., an intrinsic property of polyethylenimine (PEI). The proposed mechanism relies on the presence of basic groups, which are unprotonated at physiological pH, but can get protonated in the mildly acidic endosomal environment. The aminoethylene motifs contained in PEI or structurally related reagents provide a favorable combination of positive charge at neutral pH for electrostatic binding of nucleic acids as well as additional protonation capacity at endosomal pH.

Sequence-defined oligomers with a favorable stability, biocompatibility, and toxicity profile have been generated previously (Schaffert, D. et al., Angew. Chem., Int. Ed. 2011, 50 (38), 8986-8989; Schaffert, D. et al, Org. Lett., 2011, 13, 1586-9). WO 2011/154331 describes various oligo(alkylamino) acids for siRNA delivery in vivo. However, the drug substance affects the suitability of the oligo(alkylamino) acids as well as the sequence and shape of the oligomer or lipo-oligomer. For instance, ribonucleoprotein (RNP) complexes comprising a guide RNA (gRNA) or single guide RNA (sgRNA) bound by a positively charged Cas protein are not comparable to RNA (such as siRNA), plasmid DNA (pDNA) or artificial antisense oligonucleotides (such as phosphorodiamidate morpholino oligomers (PMOs)) as cargos. Further, lipo-oligomers comprising oligo(alkylamino) acids were generated as a delivery platform for co-delivery of the Cas9 protein and sgRNA, disclosing a T-shape lipid-oligomer comprising a headgroup with two succinyl-tetraethylenepentamines (Stp) on either side of the central lysine and a tail comprising hydroxysteric acid (Kuhn, J. et al., Delivery of Cas9/sgRNA Ribonucleoprotein Complexes via Hydroxystearyl Oligoamino Amides, Bioconjugate Chemistry, 2020, 31, 729-742). The artificial amino acid Stp complexes the negatively charged RNP complex and facilitates its cellular delivery.

However, there is the need for further improved cargo-specific optimized lipo-oligomers for delivery of Cas9/sgRNA complexes, as well as for delivery of siRNA and PMOs.

### SUMMARY OF THE INVENTION

In the present invention it was found that more hydrophobic lipo-oligomers are advantageous for Cas protein/gRNA RNP complexes, which also turned out to be advantageous for siRNA and PMO delivery. More specifically, the present invention relates to a nanoparticle for Cas protein/gRNA ribonucleoprotein (RNP) complex (cargo) delivery comprising one or more Cas protein/gRNA RNP complex(es) as cargo; and a carrier comprising a sequence-defined T-shape lipo-oligomer, comprising an artificial amino acid, wherein the artificial amino acid is an oligo(alkylamino) acid, and wherein the T-shape lipo-oligomer comprises the sequence of formula I:

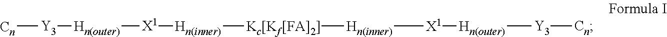

wherein
K_{c} is a central lysine;
K_{f} is a further lysine linked to the ε-amino group of K_{c};
FA is a fatty acid covalently linked to the ε- and α-amino group of K_{f};
C is cysteine and n=0 or 1, preferably n=0
Y₃ is a tyrosine tripeptide;
H is histidine and n₍ₒᵤₜₑᵣ₎ and n₍ᵢₙₙₑᵣ₎ are independently selected from 0, 1 or 2; and
X¹ is an oligo(alkylamino) acid of formula IIa:

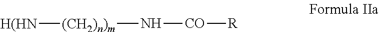

wherein n=2 or 3, alternating 2 and 3 or alternating 3 and 2; and m=3 or 4; and
wherein R is
—(CH₂)_{y}—CO₂H, wherein y=2, 3 or 4;

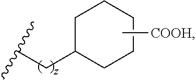

wherein z=0 or 1, preferably more preferably

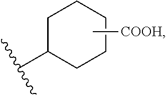

more preferably

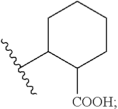 [SMILES: *C1CCCCC1C(=O)O]

—CH₂—CH(c-hexyl)-CH₂—CO₂H; or
—CH₂—NRₐ—CH₂—CO₂H, wherein Rₐ═H, CH₂—CF₃, or
wherein the T-shape lipo-oligomer comprises the sequence of formula III:

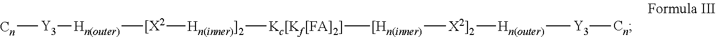

wherein
K_{c} is a central lysine;
K_{f} is a further lysine linked to the ε-amino group of K_{c};
FA is a fatty acid covalently linked to the ε- and α-amino group of Kᵢ;
C is cysteine and n=0 or 1, preferably n=1
Y₃ is a tyrosine tripeptide;
H is histidine and n₍ₒᵤₜₑᵣ₎ and n₍ᵢₙₙₑᵣ₎ are independently selected from 0 or 1; and
X² is an oligo(alkylamino) acid of formula IIa:

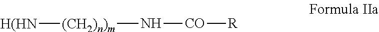

wherein n=2 or 3, alternating 2 and 3 or alternating 3 and 2; and m=3 or 4;
wherein R is
—(CH₂)_{y}—CO₂H, wherein y=3 or 4;

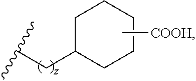

wherein z=0 or 1, preferably

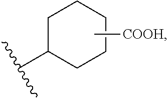

more preferably

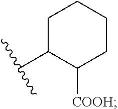 [SMILES: *C1CCCCC1C(=O)O]

—CH₂—CH(c-hexyl)—CH₂—CO₂H; or
—CH₂—NRₐ—CH₂—CO₂H, wherein Rₐ═CH₂—CF₃.

In certain embodiments, the two fatty acids are saturated or unsaturated C12 to C22 fatty acids, preferably the two fatty acids are selected from the group consisting of oleic acid (OleA), linoleic acid (LinA), linolenic acid (LenA) or hydroxy steric acid (OHSteA), preferably OleA, LinA or OHSteA, more preferably LinA or OHSteA. The oligo(alkylamino) acid is in certain embodiments a tetraethylenepentamine or a triethylenetetramine of formula IIb: H(HN—(CH₂)₂)ₘ=3 or ₄—NH—CO—R (Formula IIb); preferably a tetraethylenepentamine. The T-shape lipo-oligomer comprises in certain embodiments the sequence of formula I:

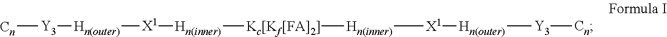

(a) wherein
   C is cysteine and n=0 or 1, preferably n=0
   Y₃ is a tyrosine tripeptide;
   H is histidine and n₍ₒᵤₜₑᵣ₎ and n₍ᵢₙₙₑᵣ₎ are 1; or
(b) wherein
   C is cysteine and n=0
   Y₃ is a tyrosine tripeptide;
   H is histidine and n₍ₒᵤₜₑᵣ₎ and n₍ᵢₙₙₑᵣ₎ are 0; or
the sequence of formula III:

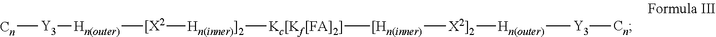

(c) wherein
   C is cysteine and n=1
   Y₃ is a tyrosine tripeptide;
   H is histidine and n₍ₒᵤₜₑᵣ₎ and n₍ᵢₙₙₑᵣ₎ are 1; or
(d) wherein
   C is cysteine and n=1
   Y₃ is a tyrosine tripeptide;
   H is histidine and n₍ₒᵤₜₑᵣ₎ and n₍ᵢₙₙₑᵣ₎ are 0.

The sequence-defined T-shape lipo-oligomer may further comprise a terminal functional group at the N-terminus or the C-terminus of the T-shape lipo-oligomer, wherein the function group is (i) an azido group, or (ii) a thiol group, provided that the sequence of formula I does not comprise a cysteine; More specifically, the sequence-defined T-shape lipo-oligomer may further comprises a terminal azido group and a targeting ligand is coupled to the azido group, preferably wherein the targeting ligand is coupled to the azido group via click chemistry, more preferably click chemistry with a dibenzocylcooctyne-coupled targeting ligand.

The RNP complex in certain embodiments comprises Cas protein/gRNA at a ratio of about 1:1 to about 1:2 and/or wherein the Cas protein/gRNA RNP complex and the sequence-defined T-shape lipo-oligomer are mixed at a lipo-oligomer nitrogen (N) to gRNA phosphate (P) ratio (N/P ratio) of about 1:12 to 1:30, preferably about 1:20 to 1:30, more preferably of about 1:22 to 1:26. The Cas protein may be (a) a Cas9, a Cas12, a Cas13 protein or an engineered variant thereof and/or (b) a base editor or a prime editor. Moreover, the one or more Cas protein/gRNA RNP complex(es) may be Cas9/gRNA RNP complex(es). The nanoparticle may further comprises two or more Cas protein/gRNA RNP complexes as cargo, wherein the two or more Cas protein/gRNA RNP complexes comprise two or more different gRNAs targeting the same or different genes, preferably different genes. In certain embodiments, the nanoparticle comprises one or more Cas protein/gRNA RNP complexes and a donor DNA.

In another aspect the invention relates to a nanoparticle for siRNA or phosphorodiamidate morpholino oligomers (PMO) (cargo) delivery comprising one or more siRNA(s) or PMO(s) as cargo; and a carrier comprising a sequence-defined T-shape lipo-oligomer comprising an artificial amino acid, wherein the artificial amino acid is an oligo(alkylamino) acid, and wherein the T-shape lipo-oligomer comprises the sequence of formula I:

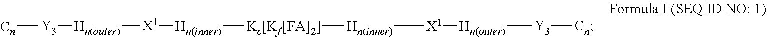

wherein
K_{c} is a central lysine;
K_{f} is a further lysine linked to the ε-amino group of K_{c};
FA is a fatty acid covalently linked to the ε- and α-amino group of Kᵣ;
C is cysteine and n=0 or 1, preferably n=0
Y₃ is a tyrosine tripeptide;
H is histidine and n₍ₒᵤₜₑᵣ₎ and n₍ᵢₙₙₑᵣ₎ are independently selected from 0, 1 or 2; and
X¹ is an oligo(alkylamino) acid of formula IIIa:

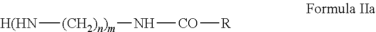

wherein n=2 or 3, alternating 2 and 3 or alternating 3 and 2; and m=3 or 4;
wherein R is
—(CH₂)_{y}—CO₂H, wherein y=2, 3 or 4;

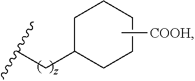

wherein z=0 or 1, preferably

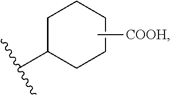

more preferably

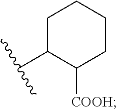 [SMILES: *C1CCCCC1C(=O)O]

—CH₂—CH(c-hexyl)-CH₂—CO₂H; or
—CH₂—NRₐ—CH₂—CO₂H, wherein Rₐ═H, CH₂—CF₃,
wherein the two fatty acids are saturated or unsaturated C12 to C22 fatty acids, preferably the two fatty acids are selected from the group consisting of oleic acid (OleA), linoleic acid (LinA), linolenic acid (LenA) or hydroxy steric acid (OHSteA), preferably OleA, LinA or OHSteA, more preferably LinA or OHSteA.

In yet another aspect the invention also relates to a conjugate comprising a phosphorodiamidate morpholino oligomer (PMO) covalently linked to a carrier comprising a sequence-defined T-shape lipo-oligomer, comprising an artificial amino acid, wherein the artificial amino acid is an oligo(alkylamino) acid, and wherein the T-shape lipo-oligomer comprises the sequence of formula I:

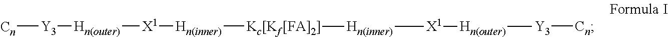

wherein
K_{c} is a central lysine;
Kᵣ is a further lysine linked to the ε-amino group of K_{c};
FA is a fatty acid covalently linked to the ε- and α-amino group of K_{f},
C is cysteine and n=0 or 1, preferably n=0
Ys is a tyrosine tripeptide;
H is histidine and n₍ₒᵤₜₑᵣ₎ and n₍ᵢₙₙₑᵣ₎ are independently selected from 0, 1 or 2; and
X¹ is an oligo(alkylamino) acid of formula IIIa:

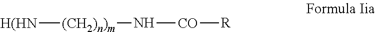

wherein n=2 or 3, alternating 2 and 3 or alternating 3 and 2; and m=3 or 4; and
wherein R is
(CH₂)_{y}—CO₂H, wherein y=2, 3 or 4;

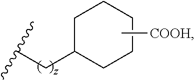

wherein z=0 or 1, preferably

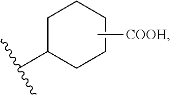

more preferably

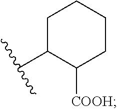 [SMILES: *C1CCCCC1C(=O)O]

—CH₂—CH(c-hexyl)-CH₂—CO₂H; or
—CH₂—NRₐ—CH₂—CO₂H, wherein Rₐ═H, CH₂—CF₃, and
wherein the two fatty acids are saturated or unsaturated C12 to C22 fatty acids, preferably the two fatty acids are selected from the group consisting of oleic acid (OleA), linoleic acid (LinA), linolenic acid (LenA) or hydroxy steric acid (OHSteA), preferably OleA, LinA or OHSteA, more preferably LinA or OHSteA.

In a further aspect, the invention relates to the nanoparticle of the invention for use in therapy.

In yet another aspect, the invention relates to the nanoparticle of the invention for use in treating cancer, a genetic disease, an infectious disease a cardiovascular disease, a metabolic disease, a neurodegenerative or neuromuscular disease, a hematological disease, a hereditary eye disease or an autoimmune disease or an autoimmune disease.

In yet another aspect, the invention relates to an in vitro method for transfecting mammalian cells with one or more Cas protein/gRNA complex(es) comprising contacting a mammalian cell in vitro with the nanoparticle according to the invention.

In yet another aspect, the invention relates to a use of the sequence-defined T-shape lipo-oligomer for cellular delivery of a Cas protein/gRNA ribonucleoprotein (RNP) complex into a target cell, wherein the sequence defined T-shape lipo-oligomer is as defined for the nanoparticles according to the invention.

In yet another aspect, provided is the use of an artificial amino acid in a sequence-defined T-shape lipo-oligomer for cellular delivery of a Cas protein/gRNA ribonucleoprotein (RNP) complex into a target cell, wherein the artificial amino acid is an oligo(alkylamino) acid selected from the group consisting of Gtp, Gtt, Htp, Ptp, IDAtp, TFE-IDAtp, GEIPA, Ntp, and chGtp. Preferably, the oligo(alkylamino) acid is selected from the group consisting of Gtp, Gtt, Htp, TFE-IDAtp, GEIPA, and chGtp, preferably Htp, TFE-IDAtp, and chGtp. The target cell is preferably a mammalian cell, such as a cell line, e.g., CHO cell or a HEK 293 cell or a primary cell in cell culture (in vitro).

In yet another aspect, an artificial amino acid is provided, wherein the artificial amino acid is an oligo(alkylamino) acid selected from the group consisting of the following formulas:

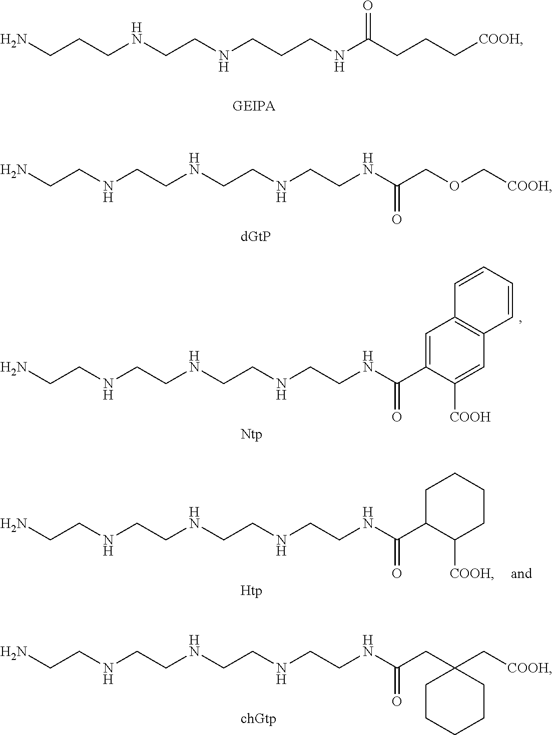 [SMILES: NCCCNCCNCCCNC(=O)CCCC(=O)O ; NCCNCCNCCNCCNC(=O)COCC(=O)O ; NCCNCCNCCNCCNC(=O)c1cc2ccccc2cc1C(=O)O ; NCCNCCNCCNCCNC(=O)C1CCCCC1C(=O)O ; NCCNCCNCCNCCNC(=O)CC1(CC(=O)O)CCCCC1]

in its protected and unprotected form.

The use is for (in vitro) cellular delivery of a Cas protein/gRNA ribonucleoprotein (RNP) complex into a target cell.

### DESCRIPTION OF THE FIGURES

FIG. 1. Schematic illustration of oligomers with optimized sequences comprising (A) one (1 Stp Series; from top to bottom SEQ ID NOs: 103, 102, 101, 100) or (B) two consecutive artificial amino acids (2 Stp Series, from top to bottom SEQ ID NOs: 107, 106, 105, 104) showing the lead compound succinyl-tetraethylenepentamine (Stp) on either side of the central lysine K as a representative artificial amino acid; (C) chemical structure of sequence defined T-shape lipo-oligomers showing Stp as one representative artificial amino acid.

FIG. 2. eGFP knockout experiments illustrating the effect of sequence optimization on Stp-based oligomers. (A) The percentage of GFP knockout in HeLa eGFP/tub cells was determined using a FACS analysis after treatment with Cas9 RNP nanoparticles at 75 nM RNP dose for 48 h. Shown is the eGFP knockout efficiency (%) and cell viability using the abbreviations of Table 1. (B) The percentage of GFP knockout in HeLa eGFP/tub cells was determined using a FACS analysis after 48 h treatment with Cas9 RNP nanoparticles at a series of RNP concentrations at concentrations ranging from 0.1 to 100 nM using the sequence-based oligomers as indicated by oligomer ID. Shown is the eGFP knockout efficiency (%) versus the ribonucleoprotein (RNP) concentration (nM). Data are presented as mean±SD (n=3).

FIG. 3. Schematic illustration of sequence-defined lipo-oligomers based on α-amino acids cysteine (C), tyrosine (Y), lysine (K), histidine (H) and the artificial oligoamino acid succinyl tetraethylenepentamine (Stp).

FIG. 4. Chemical formulas of artificial amino acid building blocks as Stp analogs in their unprotected (A) and their protected forms (B).

FIG. 5. eGFP knockout-experiments illustrating the effect of optimized artificial amino acid building blocks on lipo-oligomers with 1445-based structure. The percentage of GFP knockout in HeLa eGFP/tub cells was determined using a FACS analysis after 48 h treatment with Cas 9 RNP nanoparticles at a series of RNP concentrations ranging from 0.1 nM to 100 nM. Shown is the eGFP knockout efficiency (%) versus the ribonucleoprotein (RNP) concentration (nM).

FIG. 6. eGFP knockout-experiments illustrating the effect of optimized artificial amino acid building blocks on oligomers with 1392-based structure. The percentage of GFP knockout in HeLa eGFP/tub cells was determined using a FACS analysis after 48 h treatment with Cas 9 RNP nanoparticles at a series of RNP concentrations ranging from 0.1 nM to 100 nM. Shown is the eGFP knockout efficiency (%) versus the ribonucleoprotein (RNP) concentration (nM).

FIG. 7. eGFP knockout-experiments illustrating the effect of optimized artificial amino acid building blocks on oligomers with 1396-based structure. The percentage of GFP knockout in HeLa eGFP/tub cells was determined using a FACS analysis after 48 h treatment with Cas 9 RNP nanoparticles at a series of RNP concentrations ranging from 0.1 nM to 100 nM. Shown is the eGFP knockout efficiency (%) versus the ribonucleoprotein (RNP) concentration (nM).

FIG. 8: Plot of eGFP knockout EC50 values versus oligomer logD7.4 values of (A) 1445-based structures (B) 1396-based structures and (C) 1392-based structures with varied artificial amino acid building blocks.

FIG. 9: Evaluation of nanoparticle size, nanoparticle stability and cellular uptake in a side-by-side comparison of selected 1445-based structures, 1396-based structures and 1392-based structures. Shown is (A) Effect of dilution on the nanoparticle size determined by DLS; value=0 indicates “not detectable”; (B) nanoparticle stability against different concentrations of NaCl (0, 0.05, 0.15, 0.25, 0.5, and 5 M); Ribogreen was used for the detection of free RNA (C) nanoparticle stability against different concentrations of heparin (0, 0.25, 0.5, 1, 2.5, and 5 IU/μg sgRNA); Ribogreen was used for the detection of free RNA; and (D) Cellular uptake of Cas9 RNP (75 nM) containing 20% ATTO647N-Cas9 and 20% ATTO488-sgRNA into HeLa cells determined by flow cytometry after 4 h incubation.

FIG. 10. (A) Gating strategy to differentiate non-edited (eGFP positive), NHEJ (eGFP negative), and HDR (BFP positive) cell populations. (B) Evaluation of editing percentage of non-homologous end joining (NHEJ) and homology-directed repair (HDR) by quantification of GFP knockout (NHEJ) and GFP-to-BFP conversion (HDR) in HeLa-GFPd2 cells treated with TFE-IDAtp1-Lin1 (ID oligomer #1740) Cas9 RNP/ssDNA nanoparticles at various concentrations and ratios of RNP/ssDNA of 1/1 (top) and 1/8 (bottom).

FIG. 11: Evaluation of non-homologous end joining (NHEJ) efficiency, homology-directed repair (HDR) efficiency, total editing efficiency and non-edited cell percentage in a side-by-side comparison of selected 1445-based structures, 1396-based structures and 1392-based structures comprising the artificial amino acids as indicated. Results are shown as (A) heat map of NHEJ efficiency in HeLa GFPd2 cells 48 h after treatment with different Cas9 RNP/ssDNA nanoparticle (fixed at ¼) formulations at varied RNP concentrations; (B) heat map of HDR efficiency in HeLa GFPd2 cells 48 h after treatment with different Cas9 RNP/ssDNA (fixed at ¼) nanoparticle formulations at varied RNP concentrations; (C) heat map of total editing efficiency in HeLa GFPd2 cells 48 h after treatment with different Cas9 RNP/ssDNA nanoparticle (fixed at ¼) formulations at varied RNP concentrations; (D) heat map of non-edited cell percentages in HeLa GFPd2 cells 48 h after treatment with different Cas9 RNP/ssDNA (fixed at ¼) nanoparticle formulations at varied RNP concentrations.

FIG. 12: Structure-activity relationship of PMO (Ex23)-LP 1:3 formulations. (A) Schematic illustrations of lipo-oligomers (LP) architectures classified according to the ionizable backbone into “Stp” (#1391- #1393), “H-Stp-H” (#1395- #1397) and “Stp-Stp” (#1195). The table summarizes individual fatty acids contained. K: lysin, Y: tyrosine, Stp: succinyl tetraethylene pentamine, FA: fatty acid. (B) Splice switching activity in HeLa mCherry-DMDEx23 cells after 24 h treatment with PMO (Ex23)-LP 1:3 formulations (0.078 to 5 PM PMO). (C) Metabolic activity of HeLa mCherry-DMDEx23 cells after 24 h treatment with PMO (Ex23)-LP 1:3 formulations. (D) Dose titration of PMO (Ex23)-1195 and 1395 formulations (2.4 nM to 312.5 nM PMO) with exposure of HeLa mCherry-DMDEx23 cells for 24 h. Percentage of mCherry expressing cells was determined 24 h after treatment. Data are presented as mean±SD (n=3).

FIG. 13: Comparison of various artificial amino acids in 1392-based and 1396-based structures for PMO delivery. (A) Splice-switching activity in HeLa mCherry-DMDEx23 cells after 24 h treatment with PMO (Ex23)-LP 1:3 formulations using 1392-based oligomers. Data are presented as mean #SD (n=3). (B) Splice-switching activity in HeLa mCherry-DMDEx23 cells after 24 h treatment with PMO (Ex23)-LP 1:3 formulations using 1396-based oligomers. Data are presented as mean±SD (n=3).

FIG. 14: Gene silencing activity of siRNA polyplexes in KB/eGFPLuc cells. SiRNA polyplexes were formulated with different carriers with Stp2-HC (N/P 12), Stp1-H (N/P 24), and Stp1-HC carriers (N/P 24) in HBG (25 μg siRNA/mL), and tested at doses of 250, 125 and 63 ng siRNA per well. As siRNAs, eGFP-targeted siRNA (siGFP) and control siRNA (siCtrl) were used. Luciferase expression was measured 48 h after transfection without change of the medium. RLU was presented as percentage of the luciferase gene expression of the HBG buffer-treated cells (n=3, mean±SD).

FIG. 15: Gel electrophoresis of siRNA polyplexes in comparison to free siRNA. siCtrl polyplexes were formed with different carriers at indicated N/P ratios in HBG (25 μg siRNA/mL), and their stability was evaluated by standard agarose (2.5%, TBE buffer) gel shift assay.

### DETAILED DESCRIPTION

The term “comprises” or “comprising” means “including, but not limited to”. The term is intended to be open-ended, to specify the presence of any stated features, elements, integers, steps or components, but not to preclude the presence or addition of one or more other features, elements, integers, steps, components or groups thereof. The term “comprising” thus includes the more restrictive terms “consisting of” and “essentially consisting of”. With regard to sequences the terms “having an amino acid sequence of′ and “comprising an amino acid of” are used interchangeably and include the embodiment “consisting of the amino acid sequence of”. Similarly, the term “encoding” or “encodes” is intended to be open-ended and allows the presence or addition or one or more other features, elements or components. Furthermore, singular and plural forms are not used in a limiting way. As used herein, the singular forms “a”, “an” and “the” designate both the singular and the plural, unless expressly stated to designate the singular only.

The term “protein” is used interchangeably with “amino acid sequence” or “polypeptide” and refers to polymers of amino acids of any length. These terms also include proteins that are post-translationally modified through reactions that include, but are not limited to, glycosylation, acetylation, phosphorylation, glycation or protein processing. Modifications and changes, for example fusions to other proteins, amino acid sequence substitutions, deletions or insertions, can be made in the structure of a polypeptide while the molecule maintains its biological functional activity. For example, certain amino acid sequence substitutions can be made in a polypeptide or its underlying nucleic acid coding sequence and a protein can be obtained with the same properties. The sequence-defined (T-shape) lipo-oligomers comprising at least two artificial amino acids as used in the nanoparticle according to the invention are distinct from a polypeptide or protein.

A “peptide bond” as used herein is an amide type of covalent chemical bond linking two amino acids via the carboxylic group of one amino acid with the amino group of the other amino acid. Typically, the peptide bond refers to the bond between two a-functional groups of two amino acids between C-1 of one amino acid (carboxylate) and N of C-2 of the other amino acid (α-amino group), which may also be referred to as a-peptide bond in contrast to an amide bond with the ε-amino group of lysine (side chain amino group). For the artificial amino acids described herein the peptide bond is analogously formed e.g., between a C-terminal carboxylic group of one artificial amino acid X and the N-terminal primary amino group of the adjacent amino acid (natural or artificial).

The term “nucleic acid sequence” is used interchangeably with “polynucleotide” and refers to DNA or RNA of any length. In the context of a non-viral expression vector, particularly a plasmid and/or integration into the host cell's genome the person skilled in the art would understand that it refers to a DNA sequence or molecule. The term “pDNA” as used herein means plasmid DNA.

The term “eukaryotic cell” as used herein refers to cells that have a nucleus within a nuclear envelop and include animal cells, human cells, plant cells and yeast cells. In the present invention a “eukaryotic cell” particularly encompasses mammalian cell, such as human or rodent cells, including without being limited thereto Chinese hamster ovary (CHO) cells, Neuro-2a cells, BHK cells, HEK293 cells, HeLa cells, HepG2 cells or derivatives thereof as well as primary cells, particularly human primary cells. Mammalian cells as used herein refer to all cells of mammalian origin, such as human or rodent cells.

The term “about” as used herein refers to a variation of 10% of the value specified, for example, about 50% carries a variation from 45 to 55%.

The term “sequence-defined” as used herein refers to a sequential assembly of the compounds, particularly using a solid-phase supported synthesis, such as common Fmoc solid-phase synthesis (SPS). In the present context this involves the use of artificial amino acids with appropriate protecting groups, such as compatible with Fmoc SPS together with Fmoc α-amino acids.

The term “guide RNA” abbreviated to gRNA as used herein refers to an RNA that is partially complementary to a target DNA locus and guides the Cas protein endonuclease to this site. The gRNA may be a CRISPR RNA (crRNA), a crRNA that pairs with trans-activating crRNAs (tracrRNA), an artificial single-guide RNA, an artificial prime editing guide RNA (pegRNA) or other RNA molecules which form a complex with a Cas protein and guide it to the target DNA sequence. The term “single-guide RNA” abbreviated to sgRNA as used herein refers to an artificial RNA consisting of tracr RNA, crRNA and an artificial RNA linker. It also includes modified/improved sgRNAs, such as tru-gRNA, using a spacer sequence with <20 nucleotides complementary to the protospacer target, and hp-sgRNA, comprising an extension on the 5′end of the spacer. Wherein modified/improved means compared to the canonical guide RNA. Many Cas12 nucleases are guided by a single crRNA. The term “artificial” in the context of an RNA means an engineered non-naturally occurring RNA. A guide RNA may also be chemically modified to increase stability, reduce TLR activation and increase specificity.

The binding of the gRNA to the Cas protein results in the formation of a ribonucleoprotein (RNP) complex. The CRISPR/Cas system composed of the gRNA and a Cas protein as a targeted nuclease can identify a targeting sequence next to a protospacer adjacent motif (PAM) through guidance by a gRNA which is specific for the targeting sequence and then cleave the DNA (or RNA in specific cases) at specific sites. The gRNA therefore confers sequence specificity to the RNP complex and several gRNA (with different target specificity) can be used with the CRISPR/Cas system. In a preferred embodiment the gRNA is a sgRNA.

The terms “Cas” and “Cas protein” are used interchangeably herein and refers to a CRISPR-associated endonuclease. Suitable Cas proteins include, without being limited thereto, type II Cas proteins, e.g., Cas9 (such as SpCas9, SaCas9, CjCas9, StCas9 or NmeCas9); type V Cas proteins, e.g., Cas12a (formerly Cpf1), Cas12f (formerly Cas14), Cas12b (formerly c2c1), Cas12i, Cas12e (formerly CasX) or Cas12g; and type VI Cas proteins, e.g., Cas13a, all of which include engineered variants thereof (engineered Cas variants). Engineered Cas variants include, without being limited thereto, variants with altered PAM compatibilities, such as less restrictive or different PAM compatibility of Cas9 or Cas12 variants (e.g., Anzalone et al., Nature Biotechnology, 38, 2020: pages 824-844, supplementary Table 1); variants with higher DNA specificity, such as variants with reduced off-target Cas nuclease activity (e.g., eSpCas (1.1), SpCas9-HF1, HypaCas9, evoCas9, Sniper-Cas9, HiFiCas9, enAsCas12a-HF1); base editors (e.g., as listed in Anzalone et al., Nature Biotechnology, 38, 2020: pages 824-844, supplementary Tables 2 and 3); CRISPR-associated transposases and engineered Cas-domain-fused transposase and recombinase systems; and prime editors, as well as mutated or truncated variants, such as Cas nickase and dCas. For example, a Cas nickase (e.g., nickase Cas9n and Cas9D10A) comprising an inactivating mutation in one or more of the nuclease domains (cleaving only one of the DNA strands) and a nuclease-deficient dCas mutant with only sgRNA binding ability, optionally further fused to another enzyme, expanded the conventional editing applications. A Cas protein ortholog refers to one of two or more homologous Cas proteins derived from different species, for example Cas9 orthologs include, without being limited thereto, Cas9 protein derived from a different bacterial species, such as SpCas9 derived from Streptococcus pyogenes, SaCas9 derived from Staphylococcus aureus, CjCas9 derived from Campylobacter jejuni, StCas9 derived from Streptococcus thermophilus, and NmeCas9 from Neisseria meningitidis. Cas orthologs typically differ in the recognized PAM sequences and size. The most often used Cas9 protein is SpCas9.

The term “engineered” in the context of a protein, particularly a Cas protein, means an artificial, non-naturally protein, particularly Cas protein, such as a protein with a deleted domain and/or a fusion protein and/or a mutated protein, wherein the mutation may for example result in a different specificity, e.g., a different PAM specificity, or an inactivated or enhanced enzyme activity of the protein or of one or more of the distinct nuclease domain(s) (e.g., RuvC and/or HNH of Cas9).

The term “Cas9 protein” as used herein refers to Cas9 nucleases that are guided by guide RNAs to generate predominantly blunt-end DSBs using two distinct nucleases (RuvC and HNH), as well as engineered variants thereof, e.g., Cas9 nickase comprising an inactivated HNH and/or RuvC nuclease domain and the nuclease-deficient dCas9.

Typically, a double-strand break at the target site in the cellular genome is introduced. Strand breaks can be repaired by non-homologous end joining (NHEJ), which can introduce insertions or deletions (indels) or in the presence of a donor DNA by homology-directed repair (HDR). The donor DNA may be double-stranded DNA (dsDNA) or single-stranded DNA (ssDNA), such as single stranded oligonucleotide donors (ssODNs). It can be delivered as plasmid, linear double-stranded DNA or single stranded DNA. Also, the donor DNA may be co-delivered together with the RNP complex or may be delivered separately by non-viral or viral delivery. For example, the donor DNA may be delivered using a separate nanoparticle, wherein the carrier may be the same, i.e., the sequence-defined T-shape lipo-oligomer according to the invention or the carrier may be different. Exemplary viral-delivery methods include, e.g., adeno-associated virus (AAV), lentivirus or adenovirus, preferably AAV.

The term “indel” or “indels” as used herein refers to a variety of insertions and deletions, typically introduced by error-prone non-homologous end joining processes during the cellular repair of double-stranded DNA breaks (DSBs). Indel products that result from DSB cannot be controlled, but are not random. In open reading frames they usually generate frameshift mutations in coding sequences that abrogate protein function.

The term “chimeric single-guide RNA” abbreviated to cgRNA as used herein refers to a modified sgRNA which carries a first sequence to generate double-stranded breaks and a second sequence for homology-directed repair.

The term “base editor” as used herein introduces targeted point mutations without the requirement of DSBs or donor DNA template. Typically, a base editor containing a catalytically impaired CRISPR-Cas nuclease (that cannot make DSBs), such as Cas9 nickases or dCas9, fused to a DNA deaminase enzyme. Two main classes of base editors have been developed, cytosine base editors (CBEs), which catalyze the conversion from C/G base pairs to T/A base pairs (cytidine deaminase); and adenine base editors (ABEs), which catalyze conversion from A/T base pairs to G/C base pairs (adenosine deaminase). The base editor may optionally further be fused to proteins that modify the DNA repair machinery, (e.g., uracil glycosylase inhibitor domain (UGI) for CBEs or N-methylpurine DNA glycosylase for ABEs. Most base editors, such as CBEs BE3, BE4, BE4max and ABEs ABE7.10, ABEmax use a Cas nickase. The person skilled in the art understands that the Cas nickase nicks the non-deaminated DNA strand. Alternatively, a base editor may comprise a dCas mutant.

The term “prime editor” as used herein refers to a a combination of a Cas9 nickase domain (inactivated HNH nuclease) and an engineered reverse transcriptase domain, which may be fused or untethered. Prime editors can introduce all possible types of point mutations, including all base pair conversions, small insertions and small deletions in a precise and targeted manner with favorable editing to indel ratios. The prime editor is targeted to the editing site by an engineered prime editing guide RNA (pegRNA), which specifies the target site in its spacer sequence and the desired edit in an extension that is typically at the 3′end of the pegRNA. Upon targeted binding, the Cas9 RuvC nuclease domain nicks the PAM-containing DNA strand and uses the newly liberated 3′ end at the target DNA site to prime reverse transcription using the extension of the pegRNA. Successful priming requires that the extension in the pegRNA contain a primer binding sequence (PBS) that hybridizes with the 3′end of the nicked target DNA strand to form a primer-template complex. The reverse transcriptase domain then copies the template from the pegRNA extension into the genomic DNA directly adding the edited sequence to the target locus. The edited 3′flap replaces the redundant 5′flap, presumably by cellular DNA repair processes. Finally, the non-edited complementary strand is replaced by DNA repair using the edited strand as a template. This is facilitated by the addition of a sgRNA to nick the non-edited strand, which stimulates resynthesis of the non-edited strand using the edited strand as a template, resulting in a double-stranded edited DNA. Known prime editors, without being limited thereto are PE1 (fusion of Cas9 nickase to wild-type Moloney murine leukemia virus (M-MLV) reverse transcriptase (RT), PE2 (fusion of Cas9 nickase to engineered pentamutant M-MLV RT with increased editing efficiency), PE3 (PE2 and pegRNA and additional sgRNA), PE3b (PE3 using a nicking sgRNA that targets only the edited sequence), PE4 (PE2 in combination with DNA mismatch repair inhibiting protein MLH1dn), PE5 (PE3 in combination with MLH1dn) and PEmax (optimization of PE2) or split Prime editors, such as Split-PE (Cas9 nickase and reverse transcriptase are expressed separately and combined at the mRNA or protein level). The use of prime editors is known in the art, such as derivable from Anzalone et al., (Nature Biotechnology, 38, 2020: pages 824-844), Chen et al., (Cell, 184 (22), 2021: pages 5635-5652.e29), Liu, B. et al., (Nat Biotechnol 40, 2022:1388-1393) and Grünewald, J. et al., (Nat Biotechnol (2022); doi: 10.1038/s41587-022-01473-1).

The term “click chemistry” as used herein refers to a class of highly specific, in many cases biorthogonal, covalent conjugation reactions, that are modular, efficient, relatively insensitive to solvent parameters, water and oxygen. Typical click reactions, without being limited thereto are copper-catalyzed azide-alkylene cycloaddition (CuAAC) (copper-catalyzed reaction of an azide with an alkyne), copper-free azide-alkyne cycloaddition, such as strain-promoted azide-alkyne cycloaddition (SPAAC), Diels-Alder or inverse electron Diels-Alder reaction, thiol-ene or thiol-yne reaction, and alkene-tetrazole photoclick reaction.

The term “targeting ligand” as used herein refers to a ligand that binds to a receptor resulting in receptor-mediated endocytosis. Coupling a targeting ligand to the nanoparticles of the invention, or more specifically to the carrier comprised in the nanoparticle of the invention, allows targeted delivery and hence receptor or even cell specific delivery. For example, by incorporating an azido functional group or a thiol group into the sequence-defined T-shape lipo-oligomer (carrier) the nanoparticle can be converted to a receptor-targeted nanoparticle, e.g, via copper-free click chemistry for reacting azido functional groups with dibenzocyclooctyne (DBCO)-containing targeting ligands or thiol chemistry including thiol-maleimide addition for coupling targeting ligands such as folic acid (FolA)-PEG for folate receptor a (FRa)-specific delivery.

The term “carrier” as used herein relates to a non-viral carrier for the delivery of nucleic acid and/or protein. In the context of the present invention the carrier is a sequence-defined artificial polymer, more specifically a sequence-defined T-shape lipo-oligomer, that forms complexes, i.e., nanoparticles, with its cargo such as the Cas protein/gRNA ribonucleoprotein (RNP) complex. The term “nanoparticle” as used herein relates to the complex of the carrier (e.g., the sequence-defined T-shape lipo-oligomer) formed with its cargo (e.g. Cas protein/gRNA RNP complex) and can be as small as 6 nm up to several hundreds of nanometers, depending on the sequence-defined oligomer and/or the cargo. Such nanoparticles are also sometimes referred to as polyplexes.

Precise sequence-defined artificial polymers can be generated to meet the requirements for specific delivery. These sequence-defined artificial polymers may be formed by peptide-like artificial macromolecular structures comprising an artificial amino acid and one or more lipids or fatty acids (lipo-oligomer). The sequence-defined artificial polymers, such as sequence-defined T-shape lipo-oligomers, may also be referred to as “carrier” herein. According to the present invention the sequence-defined artificial polymer is a sequence-defined T-shape lipo-oligomer, wherein the peptide-like artificial macromolecule structure (oligomer) comprising an artificial amino acid forms a headgroup and two fatty acids covalently linked to a lysine form the hydrophobic tail.

In one aspect the invention relates to a nanoparticle for Cas protein/gRNA ribonucleoprotein (RNP) complex (cargo) delivery comprising one or more Cas protein/gRNA RNP complex(es) as cargo; and a carrier comprising a sequence-defined T-shape lipo-oligomer comprising an artificial amino acid, wherein the artificial amino acid is an oligo(alkylamino) acid, and wherein the T-shape lipo-oligomer comprises the sequence of formula I:

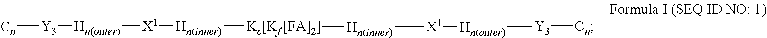

wherein
K_{c} is a central lysine;
Kᵣ is a further lysine linked to the ε-amino group of K_{c};
FA is a fatty acid covalently linked to the ε- and α-amino group of Kᵢ;
C is cysteine and n=0 or 1, preferably n=0
Y₃ is a tyrosine tripeptide;
H is histidine and n₍ₒᵤₜₑᵣ₎ and n₍ᵢₙₙₑᵣ₎ are independently selected from 0, 1 or 2; and
X¹ is an oligo(alkylamino) acid of formula IIa:

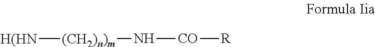

wherein n=2 or 3, alternating 2 and 3 or alternating 3 and 2; and m=3 or 4;
wherein R is
—(CH₂)_{y}—CO₂H, wherein y=2, 3 or 4;

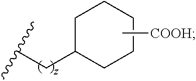

wherein z=0 or 1, preferably more preferably

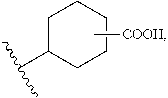

more preferably

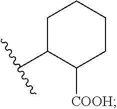 [SMILES: *C1CCCCC1C(=O)O]

—CH₂—CH(c-hexyl)-CH₂—CO₂H; or
—CH₂—NRₐ—CH₂—CO₂H, wherein Rₐ═H, CH₂-CFs, or
wherein the T-shape lipo-oligomer comprises the sequence of formula III:

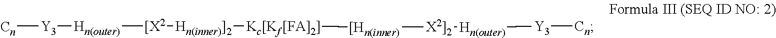

wherein
K_{c} is a central lysine;
Kᵣ is a further lysine linked to the ε-amino group of K_{c},
FA is a fatty acid covalently linked to the ε- and α-amino group of K_{f},
C is cysteine and n=0 or 1, preferably n=1
Y₃ is a tyrosine tripeptide;
H is histidine and n₍ₒᵤₜₑᵣ₎ and n₍ᵢₙₙₑᵣ₎ are independently selected from 0 or, 1; and
X² is an oligo(alkylamino) acid of formula IIa:

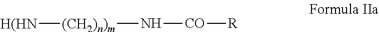

wherein n=2 or 3, alternating 2 and 3 or alternating 3 and 2; and m=3 or 4;
wherein R is
—(CH₂)_{y}—CO₂H, wherein y=3 or 4;

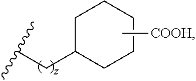

wherein z=0 or 1, preferably

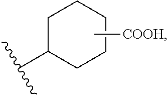

more preferably

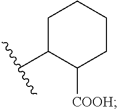 [SMILES: *C1CCCCC1C(=O)O]

—CH₂—CH(c-hexyl)-CH₂—CO₂H; or
—CH₂—NRₐ—CH₂—CO₂H, wherein Rₐ═CH₂—CF₃.

In an alternative aspect the invention relates to a nanoparticle for Cas protein/gRNA ribonucleoprotein (RNP) complex (cargo) delivery comprising one or more Cas protein/gRNA RNP complex(es) as cargo; and a carrier comprising a sequence-defined T-shape lipo-oligomer comprising
(a) a headgroup comprising an oligomer comprising
i. a central lysine (K_{c}), and
ii. an N-terminal peptide chain and a symmetrical C-terminal peptide chain referred to the central lysine, each comprising an artificial amino acid, wherein the artificial amino acid is an oligo(alkylamino) acid; and
(b) a hydrophobic tail comprising
i. two fatty acids (FA) covalently linked to the ε- and α-amino group of a further lysine (K_{f})
ii, wherein Kᵣ is linked to the ε-amino group of the central lysine (K_{c});
wherein the T-shape lipo-oligomer comprises the sequence of formula I:

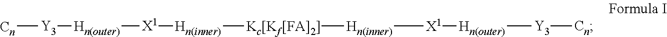

wherein
C is cysteine and n=0 or 1, preferably n=0
Y₃ is a tyrosine tripeptide;
H is histidine and n₍ₒᵤₜₑᵣ₎ and n₍ᵢₙₙₑᵣ₎ are independently selected from 0, 1 or 2; and
X¹ is an oligo(alkylamino) acid of formula IIIa:

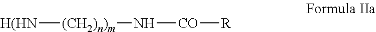

wherein n=2 or 3, alternating 2 and 3 or alternating 3 and 2; and m=3 or 4;
wherein R is
(CH₂)_{y}—CO₂H, wherein y=2, 3 or 4;

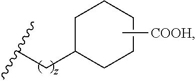

wherein z=0 or 1, preferably

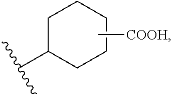

more preferably

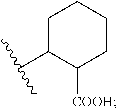 [SMILES: *C1CCCCC1C(=O)O]

—CH₂—CH(c-hexyl)-CH₂—CO₂H; or
—CH₂—NR—CH₂—CO₂H, wherein R═H, CH₂—CF₃; or
wherein the T-shape lipo-oligomer comprises the sequence of formula III:

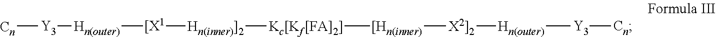

wherein
C is cysteine and n=0 or 1, preferably n=1
Y₃ is a tyrosine tripeptide;
H is histidine and n₍ₒᵤₜₑᵣ₎ and n₍ᵢₙₙₑᵣ₎ are independently selected from 0 or 1; and
X² is an oligo(alkylamino) acid of formula IIa:

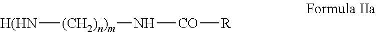

wherein n=2 or 3, alternating 2 and 3 or alternating 3 and 2; and m=3 or 4;
wherein R is
—(CH₂)_{y}—CO₂H, wherein y=3 or 4;

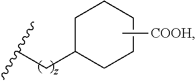

wherein z=0 or 1, preferably

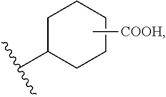

more preferably

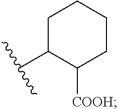 [SMILES: *C1CCCCC1C(=O)O]

—CH₂—CH(c-hexyl)-CH₂—CO₂H; or
—CH₂—NRₐ—CH₂—CO₂H, wherein Rₐ═CH₂—CF₃.

In yet another related aspect, the invention relates to a sequence-defined T-shape lipo-oligomer comprising the sequence of formula I:

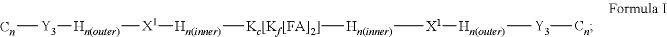

as disclosed and specified herein, or the sequence of formula III:

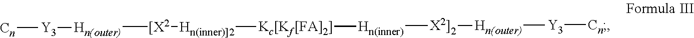

as disclosed and specified herein. The embodiments as specified for the nanoparticle of the invention similarly apply to this aspect. Preferably the sequence-defined T-shape lipo-oligomer is used as carrier for Cas protein/gRNA ribonucleoprotein (RNP) complex delivery as in the nanoparticles comprising one more Cas protein/gRNA RNP complex(es) as cargo according to the invention. However, the sequence-defined T-shape lipo-oligomer may also be used as carrier for delivery of nucleic acids, such as siRNA or phosphorodiamidate morpholino oligomers (PMO). The sequence-defined T-shape lipo-oligomer as carrier may form nanoparticles with the nucleic acid, such as siRNA or PMO, for delivery. Alternatively, the sequence-defined T-shape lipo-oligomer as carrier may be covalently linked to the PMO, preferably to the 5′- or 3′-end of the PMO. Preferably the sequence-defined T-shape lipo-oligomer as carrier covalently linked to the PMO comprises the sequence of formula I and/or the artificial amino acid is selected from Stp, chGtp, dGtp or TFEIDAtp. The fatty acid is preferably OleA or LinA.

### The Sequence-Defined T-Shape Lipo-Oligomer

The sequence-defined T-shape lipo-oligomer of formula I or III of the nanoparticle according to the invention is T-shaped with an oligomer comprising the artificial amino acid as the horizontal bar (headgroup) and two fatty acids forming the vertical (hydrophobic) tail. The sequence-defined T-shape lipo-oligomer comprises a central lysine (K_{c}) as branching point, with a further lysine linked to the ε-amino group of K_{c}, which provides a further branching point to the covalently linked fatty acids ([FA]2). The sequence-defined T-shape lipo-oligomer is preferably essentially symmetrical, more preferably it is symmetrical in sequence. This means the N-terminal and the C-terminal sequence of the headgroup is symmetrical referred to the central lysine (K_{c}), e.g., Y₃-X¹-K_{c}[K/[FA]]-X¹-Y₃ (SEQ ID NO: 10) or Y₃-H-X¹-H-K_{c}[K[FA]₂]-H-X¹-H-Y₃ (SEQ ID NO: 8). The sequence-defined T-shape lipo-oligomer of formula I or III of the nanoparticle according to the invention may further comprise a glycine between K_{c} and Kᵣ. Such an additional glycine is functionally inert. It therefore serves as a short spacer and has been added into some lipo-oligomers in the past to simplify synthesis.

In certain embodiments the T-shape lipo-oligomer comprises the sequence of formula I wherein C is cysteine and n=0 or 1, preferably n=0; Y₃ is a tyrosine tripeptide; and H is histidine and n₍ₒᵤₜₑᵣ₎ and n₍ᵢₙₙₑᵣ₎ are 1. In certain other embodiments the T-shape lipo-oligomer comprises the sequence of formula I wherein C is cysteine and n=0; Y₃ is a tyrosine tripeptide; and H is histidine and n₍ₒᵤₜₑᵣ₎ and n₍ᵢₙₙₑᵣ₎ are 0.

Formula I comprises (a) a central lysine; (b) two tyrosine tripeptides (YYY or Y₃), one tyrosine tripeptide at either side of the central lysine (K_{c}) and at the same distance to the central lysine; (c) two oligo(alkylamino) acids (X¹), one X¹ at either side of the central lysine (K_{c}) and at the same distance to the central lysine, wherein the oligo(alkylamino) acid has the formula IIa as disclosed herein; (d) a further lysine (Kᵣ) linked to the ε-amino group of the central lysine (K_{c}); and (e) two fatty acids (FA) covalently linked to the ε- and α-amino group of a further lysine (Kᵢ). Thus, the central lysine (K_{c}) serves as a branching point between the headgroup and the hydrophobic tail and the further lysine (Kᵣ) serves as a branching point for the covalent linkage of the two fatty acids (FA). The sequence-defined T-shape lipo-oligomers contain a single artificial amino acid at either side of the central lysine, wherein the artificial amino acid is an oligo(alkylamino) acid. Due to the symmetry of the T-shape lipo-oligomer the artificial amino acid on opposite side of K_{c}. is the same. The tyrosine tripeptides at either side of the central lysine represent a hydrophobic motif. This sequence provides a more hydrophobic lipo-oligomer and may allow for decreasing size of the sequence-defined T shape lipo-oligomer, without losing stability. Thus, in certain embodiments the T-shape lipo-oligomer comprises the sequence of

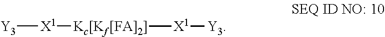

The T-shape lipo-oligomer comprising the sequence of formula I according to the invention may further comprise no, one or two histidine(s) (n=0, 1 or 2) (a) between the central lysine (K_{c}) and the oligo(alkylamino) acid (X¹) on either side of the central lysine (n (inner)=0, 1, 2); and/or (b) between the oligo(alkylamino) acid (X¹) and the tyrosine tripeptide (Y₃) on either side of the central lysine (K_{c}) (n₍ₒᵤₜₑᵣ₎=0, 1 or 2), wherein n₍ᵢₙₙₑᵣ₎ and n₍ₒᵤₜₑᵣ₎ may be independently 0, 1 or 2, preferably 0 or 1. Without being bound by theory, histidines or other imidazole derivatives with a pKa of around 6 have been incorporated into oligomers as they increase their endosomal buffer capacity, which may result in improved endosomal escape and delivery.

In certain embodiments, n₍ᵢₙₙₑᵣ₎ and/or n₍ₒᵤₜₑᵣ₎ are 0, n₍ᵢₙₙₑᵣ₎ and/or n₍ₒᵤₜₑᵣ₎ are 1, n₍ᵢₙₙₑᵣ₎ and/or n (cuter) are 2, n₍ᵢₙₙₑᵣ₎ is 0 and n₍ₒᵤₜₑᵣ₎ is 1, f (inner) is 1 and n₍ₒᵤₜₑᵣ₎ is 0, n₍ᵢₙₙₑᵣ₎ and n₍ₒᵤₜₑᵣ₎ are 0, n₍ᵢₙₙₑᵣ₎ and n₍ₒᵤₜₑᵣ₎ are 1, or n₍ᵢₙₙₑᵣ₎ and n₍ₒᵤₜₑᵣ₎ are 2; preferably n₍ᵢₙₙₑᵣ₎ and n₍ₒᵤₜₑᵣ₎ are 0, n₍ᵢₙₙₑᵣ₎ and n₍ₒᵤₜₑᵣ₎ are 1, or n₍ᵢₙₙₑᵣ₎ and n₍ₒᵤₜₑᵣ₎ are 2; more preferably n₍ᵢₙₙₑᵣ₎ and n₍ₒᵤₜₑᵣ₎ are 0, or n₍ᵢₙₙₑᵣ₎ and n₍ₒᵤₜₑᵣ₎ are 1. In certain embodiments the T-shape lipo-oligomer comprises a sequence selected from the group consisting of:

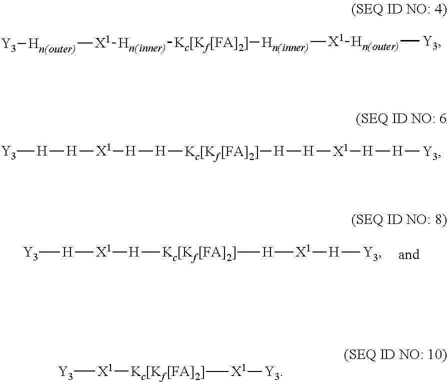

Further, cysteine (C) in formula I may be present (n=1) or absent (n=0). According to formula I cysteine is either present at the N-terminal and the C-terminal end or is absent from both ends. In a preferred embodiment cysteine is not present (n=0). Cysteine may help to stabilize the nanoparticle by formation of disulfide bonds. Particularly for less stable sequence-defined oligomers cysteines may be important. As demonstrated in the examples, the presence of cysteine is not required in small hydrophobically stabilized sequence-defined T-shape lipo-oligomers. In certain embodiments the sequence-defined T-shape lipo-oligomer comprises a sequence selected from the group consisting of:

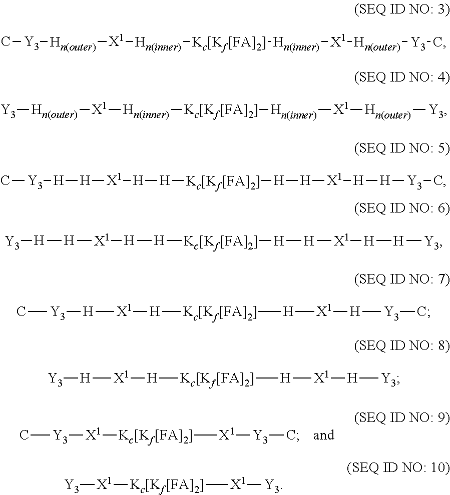

The sequence-defined T-shape lipo-oligomer comprising the sequence of formula I as disclosed herein may further comprise a terminal functional group at the N-terminus or the C-terminus of the sequence-defined T-shape lipo-oligomer, wherein the functional group is (i) an azido group, such as an azido-hexanoic acid or an azido-lysine, or (ii) a thiol group provided that the sequence of formula l does not comprise a cysteine (i.e., in formula I, n=0 for Cₙ). The functional group is, e.g., for coupling a targeting ligand. The functional group may further be used for coupling cargo, such as PMOs. The functional group (azido group or thiol group) may be N-terminally or C-terminally, preferably the functional group is N-terminally. Thus, the sequence-defined T-shape lipo-oligomer comprising the sequence of formula I may comprise a terminal azido group, such as an azido-hexanoic acid or an azido-lysine, preferably an azido-lysine. The azido group may be N-terminally or C-terminally, preferably the azido group is N-terminally. Although a single terminal azido group is sufficient, theoretically the sequence-defined T-shape lipo-oligomer may also comprise an azido group at the N-terminal and/or the C-terminal. In certain embodiments the azido-lysine (K(N₃)) comprising sequence-defined T-shape lipo-oligomer comprises a sequence selected from the group consisting of:

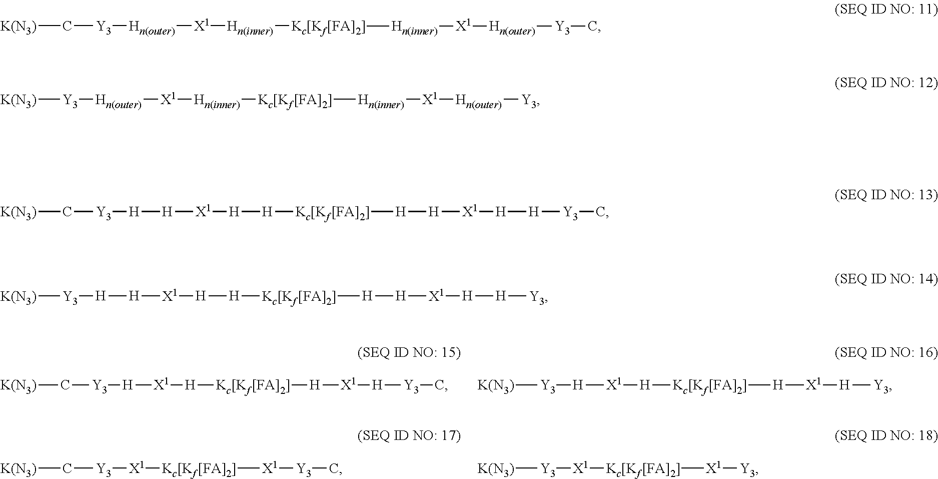

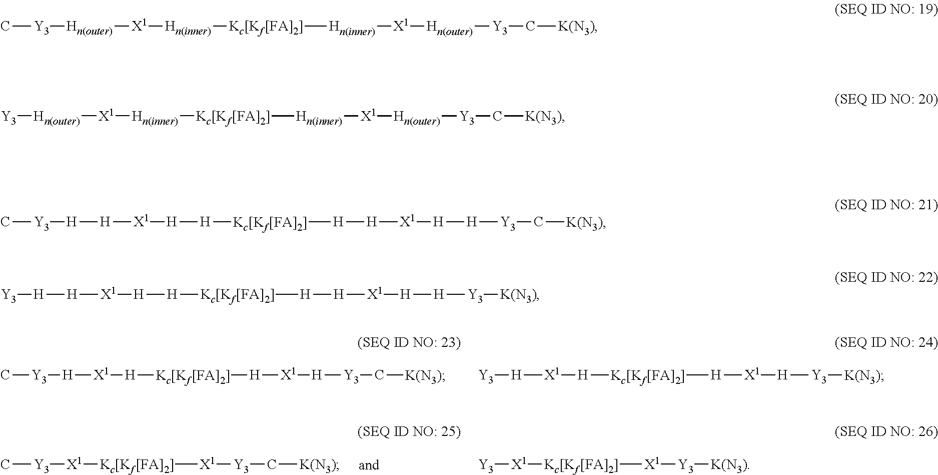

Alternatively, the sequence-defined T-shape lipo-oligomer comprising the sequence of formula I may comprise a terminal thiol group (a single terminal thiol group), provided that that the sequence of formula I does not comprise a cysteine (i.e., in formula I n=0 for Cₙ). Thus, the sequence-defined T-shape lipo-oligomer comprises a single terminal thiol group. This single terminal thiol group may be N-terminally or C-terminally, preferably the thiol group is N-terminally. Preferably the thiol group is a cysteine and the sequence-defined T-shape lipo-oligomer comprises a single cysteine at the N-terminus or the C-terminus. Thus, if the cysteine is used for coupling a targeting ligand only a single terminal cysteine should be present. In certain embodiments the single cysteine comprising sequence-defined T-shape lipo-oligomer comprises a sequence selected from the group consisting of:

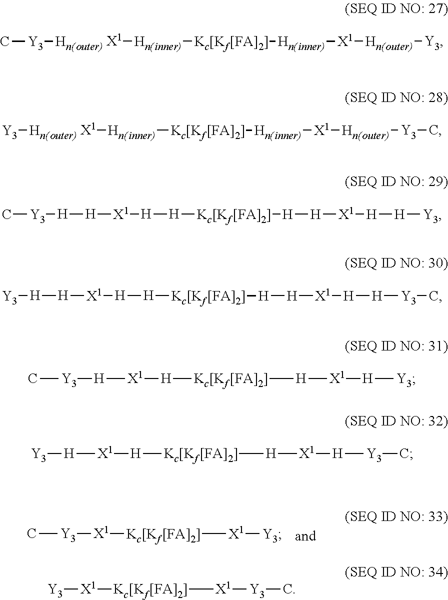

In certain embodiments the T-shape lipo-oligomer comprises the sequence of formula III wherein C is cysteine and n=0 or 1, preferably n=1; Y₃ is a tyrosine tripeptide; and H is histidine and n₍ₒᵤₜₑᵣ₎ and n₍ᵢₙₙₑᵣ₎ are 1. In certain other embodiments the T-shape lipo-oligomer comprises the sequence of formula III wherein C is cysteine and n=1; Y₃ is a tyrosine tripeptide; and H is histidine and n₍ₒᵤₜₑᵣ₎ and n₍ᵢₙₙₑᵣ₎ are 0.

Formula III comprises (a) a central lysine; (b) two tyrosine tripeptides (YYY or Y₃), one tyrosine tripeptide at either side of the central lysine (K_{c}) and at the same distance to the central lysine; (c) two oligo(alkylamino) acids (X²) at either side of the central lysine (K_{c}) and at the same distance to the central lysine, wherein the oligo(alkylamino) acid has the formula IIa as disclosed herein for formula III; (d) a further lysine (Kᵣ) linked to the ε-amino group of the central lysine (K_{c}); and (e) two fatty acids (FA) covalently linked to the ε- and α-amino group of a further lysine (K_{f}). Thus, the central lysine (K_{c}) serves as a branching point between the headgroup and the hydrophobic tail and the further lysine (Kᵣ) serves as a branching point for the covalent linkage of the two fatty acids (FA). The sequence-defined T-shape lipo-oligomers contain either (a) two consecutive artificial amino acid at either side of the central lysine, wherein the artificial amino acid is an oligo(alkylamino) acid in the absence of H_{n (inner)} (H is histidine and n₍ᵢₙₙₑᵣ₎ or n₍ₒᵤₜₑᵣ₎ and n₍ᵢₙₙₑᵣ₎ are absent (n=0)), or (b) two artificial amino acids separated by a histidine (H) at either side of the central lysine in the presence of H_{n (inner)} (H is histidine and n₍ᵢₙₙₑᵣ₎ Of n₍ₒᵤₜₑᵣ₎ and n₍ᵢₙₙₑᵣ₎ are present (n=1)). The two artificial amino acids (X²) on the same side of K_{c} may be the same or different, preferably the two artificial amino acids (X²) on the same side of K_{c} are the same. Due to the symmetry of the T-shape lipo-oligomer the artificial amino acids on opposite side of K_{c} are the same. The tyrosine tripeptides at either side of the central lysine represent a hydrophobic motif. The more hydrophobic artificial amino acids used in formula III may allow for decreasing size of the sequence-defined T shape lipo-oligomer, without losing stability. Thus, in certain embodiments the T-shape lipo-oligomer comprises the sequence of

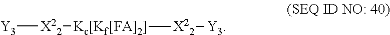

The T-shape lipo-oligomer comprising the sequence of formula III according to the invention may further comprise no or one histidine (n=0 or 1) (a) between the central lysine (K_{c}) and each of the two oligo(alkylamino) acids (X²) on either side of the central lysine (n (inner)=0 or 1); and/or (b) between the outer oligo(alkylamino) acid (X²) and the tyrosine tripeptide (Y₃) on either side of the central lysine (K_{c}) (n₍ₒᵤₜₑᵣ₎=0 or 1), wherein n₍ᵢₙₙₑᵣ₎ and n₍ₒᵤₜₑᵣ₎ may be independently 0 or 1. Preferably n₍ᵢₙₙₑᵣ₎ and n₍ₒᵤₜₑᵣ₎ are the same, both either absent or present (n (inner) and (outer)=0 or 1), more preferably n₍ᵢₙₙₑᵣ₎ and n₍ₒᵤₜₑᵣ₎ are both absent (n₍ᵢₙₙₑᵣ₎ and n₍ₒᵤₜₑᵣ₎=0). Without being bound by theory. histidines or other imidazole derivatives with a pKa of around 6 have been incorporated into oligomers as they increase their endosomal buffer capacity, which may result in improved endosomal escape and delivery.

In certain embodiments, n₍ᵢₙₙₑᵣ₎ and/or n₍ₒᵤₜₑᵣ₎ are 0, n₍ᵢₙₙₑᵣ₎ and/or n₍ₒᵤₜₑᵣ₎ are 1, Iₙ₍ᵢₙₙₑᵣ₎ is 0 and n₍ₒᵤₜₑᵣ₎ IS 1, N (inner) IS 1 and n₍ₒᵤₜₑᵣ₎ is 0, n₍ᵢₙₙₑᵣ₎ and n₍ₒᵤₜₑᵣ₎ are 0, n₍ᵢₙₙₑᵣ₎ and n₍ₒᵤₜₑᵣ₎ are 1; preferably n (inner) and n₍ₒᵤₜₑᵣ₎ are 0 or n₍ᵢₙₙₑᵣ₎ and n₍ₒᵤₜₑᵣ₎ are 1; more preferably n₍ᵢₙₙₑᵣ₎ and n₍ₒᵤₜₑᵣ₎ are 0. In certain embodiments the T-shape lipo-oligomer comprises a sequence selected from the group consisting of:

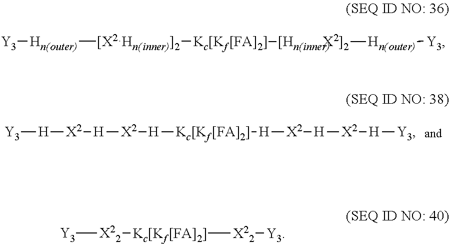

Further, cysteine (C) in formula III may be present (n=1) or absent (n=0), preferably cysteine is present. According to formula I cysteine is either present at the N-terminal and the C-terminal end or is absent from both ends. In a preferred embodiment cysteine is not present (n=0). Cysteine may help to stabilize the nanoparticle by formation of disulfide bonds. Particularly for less stable sequence-defined oligomers cysteines may be important. As demonstrated in the examples, the presence of cysteine is not required in small hydrophobically stabilized sequence-defined T-shape lipo-oligomers and may further not be necessary when using more hydrophobic artificial amino acids. In certain embodiments the sequence-defined T-shape lipo-oligomer comprises a sequence of formula III selected from the group consisting of:

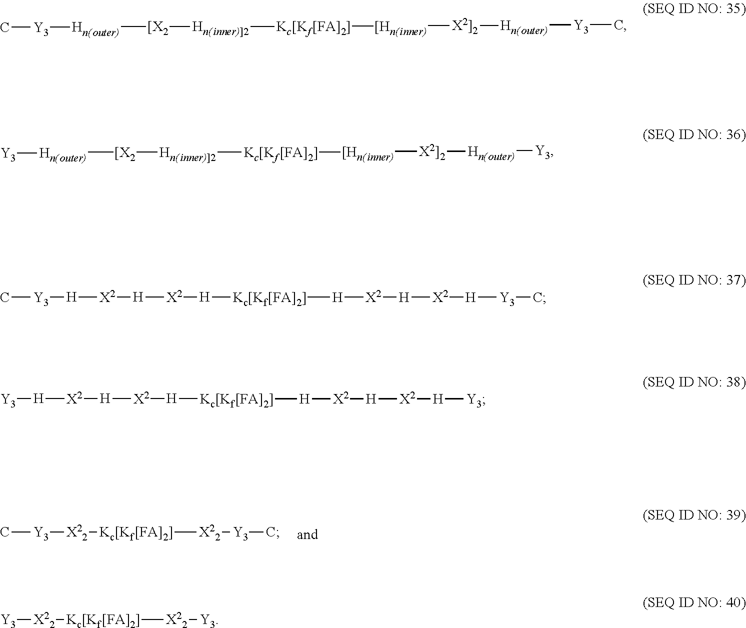

The sequence-defined T-shape lipo-oligomer comprising the sequence of formula III as disclosed herein may further comprise a terminal functional group at the N-terminus or the C-terminus of the sequence-defined T-shape lipo-oligomer, wherein the functional group is (i) an azido group, such as an azido-hexanoic acid or an azido-lysine, or (ii) a thiol group provided that the sequence of formula III does not comprise a cysteine (i.e., in formula III, n=0 for Cₙ). The functional group is, e.g., for coupling a targeting ligand. The functional group may further be used for coupling cargo, such as PMOs. The functional group (azido group or thiol group) may be N-terminally or C-terminally, preferably the functional group is N-terminally. Thus, the sequence-defined T-shape lipo-oligomer comprising the sequence of formula III may comprise a terminal azido group, such as an azido-hexanoic acid or an azido-lysine, preferably an azido-lysine. The azido group may be N-terminally or C-terminally, preferably the azido group is N-terminally. Although a single terminal azido group is sufficient, theoretically the sequence-defined T-shape lipo-oligomer may also comprise an azido group at the N-terminal and/or the C-terminal. In certain embodiments the azido-lysine (K(N₃) comprising sequence-defined T-shape lipo-oligomer comprises a sequence of formula III selected from the group consisting of:

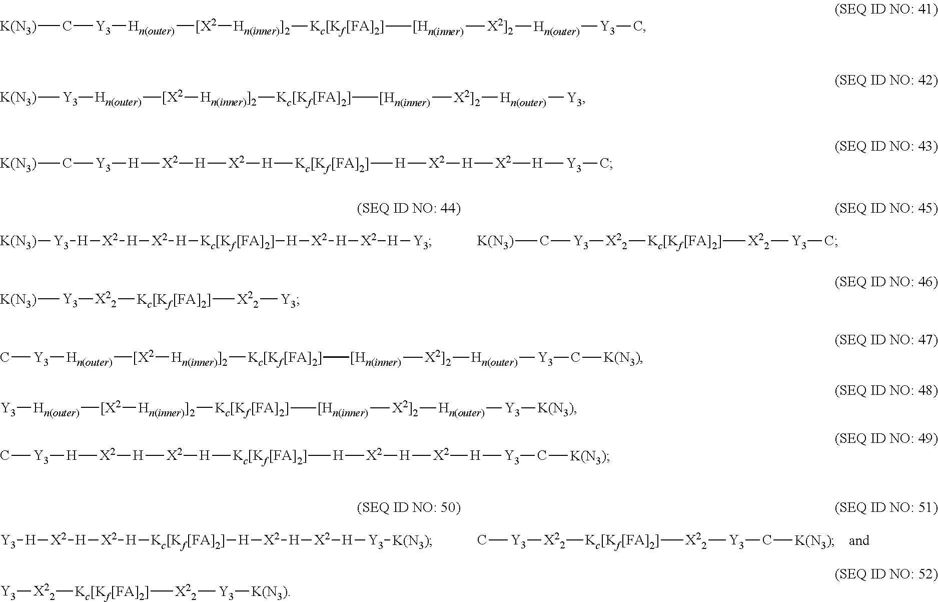

Alternatively, the sequence-defined T-shape lipo-oligomer comprising the sequence of formula III may comprise a terminal thiol group (a single terminal thiol group), provided that that the sequence of formula III does not comprise a cysteine (i.e., in formula I n=0 for Cₙ). Thus, the sequence-defined T-shape lipo-oligomer comprises a single terminal thiol group. This single terminal thiol group may be N-terminally or C-terminally, preferably the thiol group is N-terminally. Preferably the thiol group is a cysteine and the sequence-defined T-shape lipo-oligomer comprises a single cysteine at the N-terminus or the C-terminus. Thus, if the cysteine is used for coupling a targeting ligand only a single terminal cysteine should be present. In certain embodiments the single cysteine comprising sequence-defined T-shape lipo-oligomer comprises a sequence selected from the group consisting of:

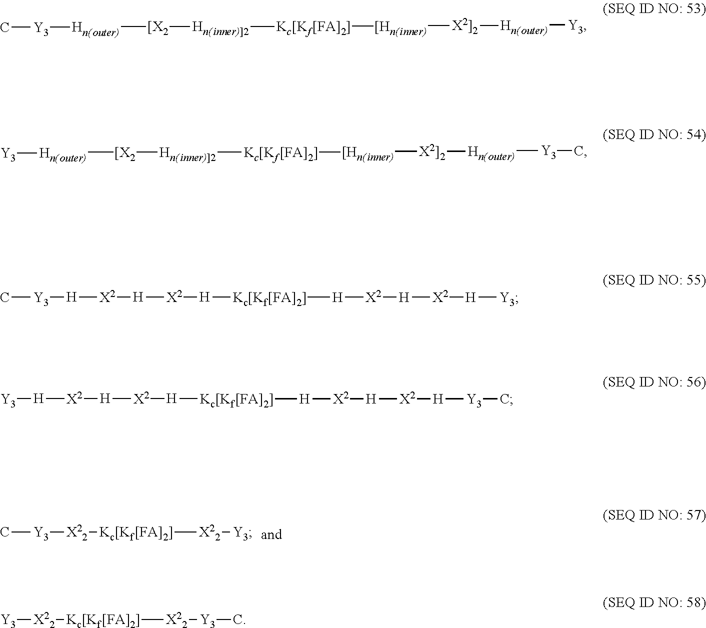

A terminal azido group or a single terminal thiol group allows coupling of, e.g., a targeting ligand using click chemistry or using thiol conjugation, respectively. The functional group may further be used for coupling cargo, such as PMOs. Thus, in certain embodiments the sequence-defined T-shape lipo-oligomer comprises a sequence of formula I further comprising a terminal azido group, such as a terminal azido-lysine or azido-hexanoic acid and a targeting ligand is coupled to the azido-lysine or azido-hexanoic acid. Preferably the terminal azido group is a terminal azido-lysine. More preferably the targeting ligand is coupled to the azido-group via click chemistry, more preferably click chemistry with a dibenzocylooctyne-coupled targeting ligand. Click chemistry is a general term for highly specific, in many cases biorthogonal, covalent conjugation reactions, that are modular, efficient and relatively insensitive to solvent parameters, water and oxygen. Various click chemistry reactions are known in the art and the person skilled in the art would know how to select a specific click chemistry reaction for a certain conjugation and particular for a certain protein or oligomer, such as the sequence-defined T-shape lipo-oligomer. Typical click reactions, without being limited thereto, are copper-catalyzed azide-alkylene cycloaddition (CuAAC) (copper-catalyzed reaction of an azide with an alkyne), copper-free azide-alkyne cycloaddition, such as strain-promoted azide-alkyne cycloaddition (SPAAC), Diels-Alder or inverse electron Diels-Alder reaction, and alkene-tetrazole photoclick reaction. Alternatively, a single terminal cysteine may be used for coupling via disulfide bond formation, thiol-maleimide or other thiol click reaction, and preferably a targeting ligand is coupled to the cysteine.

The targeting ligand may be a ligand that binds to a receptor resulting in receptor-mediated endocytosis (also referred to as receptor-mediated internalization). This may be a natural ligand or an artificial ligand, such as an antibody, a fusion protein or a small molecule binding to a receptor and mediating uptake. Coupling a targeting ligand to the carrier of the nanoparticles of the invention allows targeted delivery and hence receptor or even cell specific delivery. For example, by incorporating an azido functional group into the sequence-defined oligomer the nanoparticle can be converted to a receptor-targeted nanoparticle, e.g, via copper-free click chemistry with dibenzocyclooctyne (DBCO)-containing targeting ligands, such as folic acid (FolA)-PEG as targeting ligand for folate receptor a (FRα)-specific delivery. Alternatively, a single terminal thiol group in combination with thiol chemistry may be used for coupling targeting ligands, such as folic acid (FolA)-PEG as targeting ligand for folate receptor a (FRα)-specific delivery. For example, targeting ligands such as B6, cRGD, folic acid, methotrexate (MTX), c-Met-binding peptide (cMBP2), transferrin (Tf), AP-1, EGF, EGF receptor-binding peptide (GE11) and IL-6 receptor binding 16P7 peptide may be used. Other suitable receptors for receptor-mediated internalization and specific delivery may, e.g., be tumor antigens and the like.

The two fatty acids in the sequence-defined T-shape lipo-oligomer ([FA]₂ in formula 1) may be the same or different, preferably the two fatty acids are the same fatty acid. In certain embodiments, the two fatty acids are saturated or unsaturated C12 to C22 fatty acids, preferably the two fatty acids are linear (non-branched) saturated or unsaturated C12 or C22 fatty acids, more preferably unsaturated C14 to C20 fatty acids or saturated C12 to C18 fatty acids, more preferably unsaturated C16 to C20 fatty acids or saturated C12 to C14 fatty acids, more preferably unsaturated C18 fatty acids. In certain embodiments, the two fatty acids are selected from the group consisting of oleic acid (OleA), linoleic acid (LinA), linolenic acid (LenA) or hydroxy steric acid (OHSteA), preferably OleA, LinA or OHSteA, more preferably LinA or OHSteA In certain specific embodiments the sequence defined T-shape lipo-oligomer comprises the sequence of formula I, wherein FA is LinA, more specifically wherein (i) H is histidine and n₍ₒᵤₜₑᵣ₎ and n₍ᵢₙₙₑᵣ₎ are both 1 or 0; and (ii) C is cysteine and n=0; and (ill) FA is LinA, optionally the sequence defined T-shape lipo-oligomer comprises a terminal functional group selected from an azido group and a thiol group (single thiol group). In certain other specific embodiments, the sequence defined T-shape lipo-oligomer comprises the sequence of formula III, wherein FA is OHSteA, more specifically wherein (i) H is histidine and n₍ₒᵤₜₑᵣ₎ and n (inner) are both 1 or 0; and (ii) C is cysteine and n=1; and (ill) FA is OHSteA, optionally the sequence defined T-shape lipo-oligomer comprises a terminal azido group.

### The Artificial Amino Acid of the Sequence-Defined T-Shape Lipo-Oligomer

The sequence defined T-shape lipo-oligomer of formula I of the nanoparticle according to the invention comprises two artificial amino acids as building-block, wherein the artificial amino acid is an oligo(alkylamino) acid (X¹), one oligo(alkylamino) acid (X¹) at either side of the central lysine (K_{c}) and at the same distance to the central lysine. Thus, X¹ in formula I represents a single artificial amino acid that is an oligo(alkylamino) acid in contrast to X² (e.g., in sequence 1445) or [X²-Hₙ₍ᵢₙₙₑᵣ₎]₂ and [Hₙ₍ᵢₙₙₑᵣ₎−X²₂ as in the T-shape lipo-oligomer of formula III. X²² represents two consecutive artificial amino acids (X²₂, e.g., in sequence #1445) at either side of the central lysine, wherein the artificial amino acid is an oligo(alkylamino) acid. In these cases Hₙ₍ᵢₙₙₑᵣ₎ of formula III is absent (H is histidine and n₍ᵢₙₙₑᵣ₎ or n₍ₒᵤₜₑᵣ₎ and n₍ᵢₙₙₑᵣ₎ are absent (n=0). [X²-Hₙ₍ᵢₙₙₑᵣ₎]₂ and [Hₙ₍ᵢₙₙₑᵣ₎-X²]₂ represents two artificial amino acids separated by a histidine (H) and followed or preceded, respectively, at either side of the central lysine. In these cases Hₙ₍ᵢₙₙₑᵣ₎ is present (H is histidine and n₍ᵢₙₙₑᵣ₎ of n₍ₒᵤₜₑᵣ₎ and n₍ᵢₙₙₑᵣ₎ are present (n=1). The two artificial amino acids (X²) on the same side of K_{c} may be the same or different, preferably the two artificial amino acids (X²) on the same side of K_{c} are the same. Due to the symmetry of the T-shape lipo-oligomer the artificial amino acids on opposite side of K_{c} are the same. The oligo(alkylamino) acid is linked via its C-terminal carboxylic group and its N-terminal primary amino group. The artificial amino acid of the sequence-defined T-shape lipo-oligomer of formula I according to the present invention is an oligo(alkylamino) acid (X¹) of formula IIa:

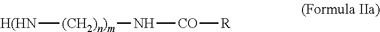

wherein n=2 or 3, alternating 2 and 3 or alternating 3 and 2; and m=3 or 4; and
wherein R is
—(CH₂)_{y}—CO₂H, wherein y=2, 3 or 4;

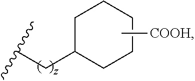

wherein z=0 or 1, preferably

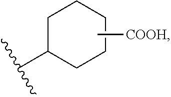

more preferably

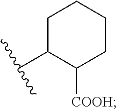 [SMILES: *C1CCCCC1C(=O)O]

—CH₂—CH(c-hexyl)-CH₂—CO₂H; or
—CH₂—NR—CH₂—CO₂H, wherein R═H, CH₂—CF₃.

The artificial amino acids in the sequence-defined T-shape lipo-oligomer of formula III according to the present invention are two oligo(alkylamino) acids (X²) of formula IIIa:

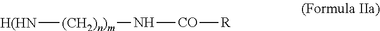

wherein n=2 or 3, alternating 2 and 3 or alternating 3 and 2; and m=3 or 4;
wherein R is
   —(CH₂)_{y}—CO₂H, wherein y=3 or 4;

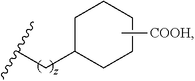

wherein z=0 or 1, preferably

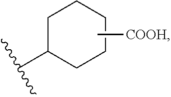

more preferably

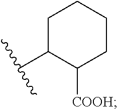 [SMILES: *C1CCCCC1C(=O)O]

—CH₂—CH(c-hexyl)-CH₂—CO₂H; or
—CH₂—NR—CH₂—CO₂H, wherein R═CH₂—CF₃.

The oligo(alkylamino) acid (X¹ and X²) of formula IIa contains 3 or 4 (m=3 or 4) protonatable alkylamino repeats, particularly 3 or 4 repeats of protonatable amino ethylene (HN—(CH₂) 2; i.e., n=2) or amino propylene motifs (HN—(CH₂)₃; i.e., n=3) or alternating amino ethylene and amino propylene motifs (n=2 and 3, preferably alternating) or vice versa. In certain embodiments n is 2, 2, 2 (for m=3) or 2, 2, 2, 2 (for m=4), or n is 3, 3, 3 (for m=3) or 3, 3, 3, 3 (for m=4), or n is 2, 3, 2 (for m=3) or 2, 3, 2, 3 (for m=4), or n is 3, 2, 3 (for m=3) or 3, 2, 3, 2 (for m=4).

According to the invention, the oligo(alkylamino) acid (of formula I and III) may be a tetraethylenepentamine or a triethylenetetramine of formula IIb:

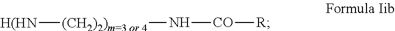

preferably a tetraethylenepentamine of formula IIc:

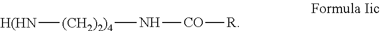

In certain embodiments, the oligo(alkylamino) acid (of formula I or III) is selected from the group consisting of the following formulas:

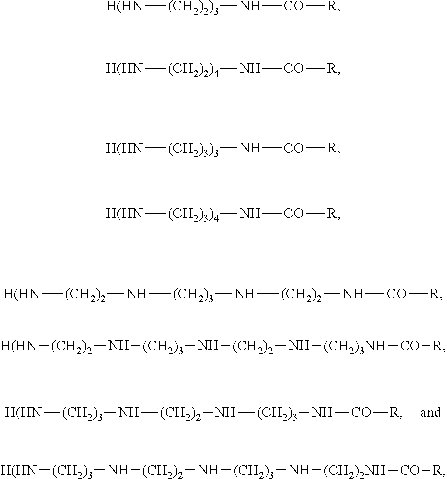

wherein R is as defined for formula IIa of formula I or III.

In the context of formula I (comprising X¹), residue R according to formula II may be —(CH₂)_{y}—CO₂H, wherein y=2, 3 or 4; preferably 3 or 4

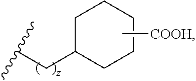

wherein z=0 or 1, preferably

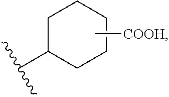

more preferably

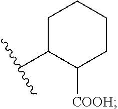 [SMILES: *C1CCCCC1C(=O)O]

—CH₂—CH(c-hexyl)-CH₂—CO₂H; or
—CH₂—NRₐ—CH₂—CO₂H, wherein Rₐ═H or CH₂—CF₃.

In a preferred embodiment the oligo(alkylamino) acid comprises a cyclohexyl group, for example R is —CH₂—CH(c-hexyl)-CH₂—CO₂H;

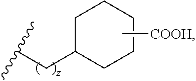

wherein z=0 or 1;

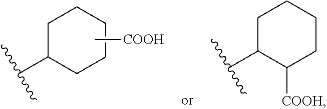

preferably —CH₂—CH(c-hexyl)-CH₂—CO₂H;

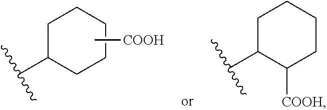

more preferably —CH₂—CH(c-hexyl)-CH₂—CO₂H or

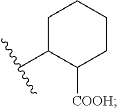 [SMILES: *C1CCCCC1C(=O)O]

or the oligo(alkylamino) acid comprises a trifluormethyl group, for example R is —CH₂—N(CH₂—CF₃)—CH₂—CO₂H. Suitable oligo(alkylamino) acids of formula IIa for the nanoparticle according to the invention comprising the T-shape lipo-oligomers comprising the sequence of formula I are, without being limited thereto, succinyl-cyclohexanedicarboxyl-tetraethylenepentamine (Htp), phthalyl-tetraethylenepentamine (Stp), tetraethylenepentamine (Ptp), naphthalenedicarboxyl-tetraethylenepentamine (Ntp), glutaryl-tetraethylenepentamine (Gtp), 1,1-cyclohexanediacetyl-tetraethylenepentamine (chGtp), iminodiacetyl-tetraethylenepentamine (IDAtp), trifluoroethyl-iminodiacetyl-tetraethylenepentamine (TFE-IDAtp), glutaryl-triethylenetetramine (Gtt), or glutaryl-N,N′-bis-(3-aminopropyl)-ethylenediamine (GEIPA) having the formula as shown below.

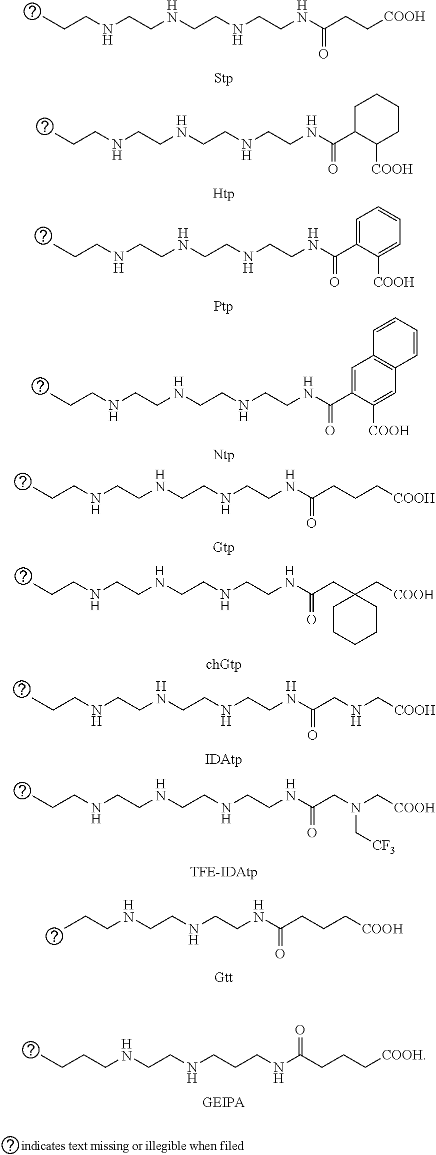

Preferably the oligo(alkylamino) acid is selected from the group consisting of Stp, Htp, Gtp, chGtp, TFE-IDAtp, Gtt and GEIPA, more preferably Htp, Gtp, chGtp and TFE-IDAtp.

In the context of formula III (comprising X²₂ or [X²-Hₙ₍ᵢₙₙₑᵣ₎] and [Hₙ₍ᵢₙₙₑᵣ₎-X²₂), the residue R according to formula IIa may be
(CH₂)_{y}-CO₂H, wherein y=3 or 4; preferably 3 or 4

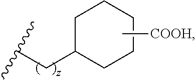

wherein z=0 or 1, preferably

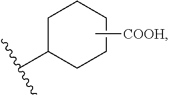

more preferably

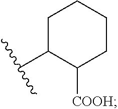 [SMILES: *C1CCCCC1C(=O)O]

—CH₂—CH(c-hexyl)-CH₂—CO₂H; or
—CH₂—NRₐ—CH₂—CO₂H, wherein Rₐ═CH₂—CF₃.

In a preferred embodiment the oligo(alkylamino) acid comprises a cyclohexyl group, for example R is —CH₂—CH(c-hexyl)-CH₂—CO₂H;

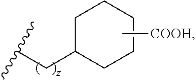

wherein z=0 or 1;

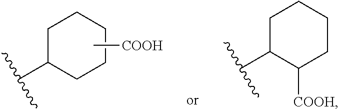

preferably —CH₂—CH(c-hexyl)-CH₂—CO₂H;

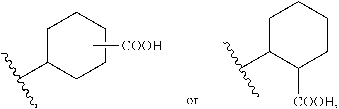

more preferably —CH₂—CH(c-hexyl)-CH₂—CO₂H or

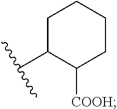 [SMILES: *C1CCCCC1C(=O)O]

or the oligo(alkylamino) acid comprises a trifluormethyl group, for example R is —CH₂—N(CH₂—CF₃)—CH₂—CO₂H. Suitable oligo(alkylamino) acids of formula IIa for the nanoparticle according to the invention comprising formula III are, without being limited thereto, chGtp, Htp, TFE-IDAtp, Gtt or GEIPA. Preferably the oligo(alkylamino) acid is TFE-IDAtp or Gtt.

An optimal sequence-defined lipo-oligomer for nanoparticle packaging and delivery depends on the cargo. For example, RNA tends to require stabilization, which may be achieved with the overall longer sequences and long fatty acids of the T-shape lipo-oligomers (head group), while DNA packaging generally is less problematic and rather cargo release inside cells can be critical, which may be achieved with shorter fatty acids and overall shorter sequences of the T-shape lipo-oligomers (headgroup). Overall, the right balance of stabilization and destabilization needs to be identified for each cargo. However, this is not trivial or predictable and sequence-defined lipo-oligomers need to be empirically tested for each cargo, and other aspects further play a role. Critical bottle-necks within the delivery pathway of the CRISPR/Cas system are the cellular uptake, endosomal release and nuclear entry. The T-shape lipo-oligomers of the nanoparticles according to the invention are particularly hydrophobic and it has been shown that more hydrophobic lipo-oligomers are advantageous for Cas protein/gRNA RNP complex delivery. It has been surprising that the short lipo-oligomers (comprising only one artificial amino acid on each site of the branching point, i.e., the central lysine) are sufficient to stabilize RNPs. Without being bound by theory, this may be due to the overall high hydrophobicity, stabilizing the nanoparticles formed with the RNP complexes. While some of the artificial amino acids used in the nanoparticles according to the invention have been used previously, they have been generated to provide basic properties, while a tuned or increased hydrophobicity of artificial amino acids has not been recognized as being an important parameter in this context.

### Ribonucleoproteins as Cargo

The nanoparticle according to the invention in certain embodiments comprises one or more Cas protein/gRNA RNP complexes as cargo. Without being limited thereto, suitable Cas protein/gRNA ratios are about 1:1 to about 1:2. Moreover, the Cas protein/gRNA RNP complex and the sequence-defined T-shape lipo-oligomer are preferably mixed at a lipo-oligomer nitrogen (N) to nucleic acid phosphate (P) (gRNA or gRNA and donor DNA) ratio (N/P ratio) of about 1:12 to 1:30, preferably of about 1:12 to 1:26. The person skilled in the art would know that only the protonatable nitrogens are considered for determining the N/P ratio. In an oligo(alkylamino) acid with five nitrogens, three are protonatable and with 4 nitrogens, two are protonatable. The nanoparticle may further comprise a donor DNA for HDR. The donor DNA may be provided as plasmid DNA, as linear double-stranded DNA or as single-stranded DNA.

The guide RNA (gRNA) is complementary to a target DNA locus and guides the Cas protein endonuclease to this site. The gRNA may be a CRISPR RNA (crRNA), a crRNA that pairs with trans-activating crRNAs (tracrRNA), an artificial single-guide RNA (sgRNA), an artificial prime editing guide RNA (pegRNA), a chimeric single-guide RNA (cgRNA) or other RNA molecules which form a complex with a Cas protein and guide it to the target DNA sequence. Preferably, the guide RNA is a single-guide RNA (sgRNA), an artificial RNA consisting of tracr RNA, crRNA and an artificial RNA linker. The single-guide RNA may also be a modified and/or improved sgRNAs, such as tru-gRNA (using a spacer sequence with <20 nucleotides complementary to the protospacer target) and hp-sgRNA (comprising an extension on the 5′end of the spacer). However, the person skilled in the art would know (see, e.g., Anzalone et al., Nature Biotechnology, 2020, 38:824-844) that the type of guide RNA may depend on the intended use and on the Cas protein used. While Cas9 proteins typically used in complex with an sgRNA, many Cas12 nucleases are guided by a single crRNA. Cas 12 nucleases possess just a single RuvC-like nuclease domain that mediates targeted DNA cleavage of both strands, typically staggered cuts within the region of the protospacer.

The Cas protein may be any CRISPR-associated endonuclease, preferably class 2 (types II, V and VI) proteins, which have single-subunit effectors. Suitable Cas proteins include, without being limited thereto, type II Cas proteins, e.g., Cas9 (such as SpCas9, SaCas9, CjCas9, StCas9 or NmeCas9); type V Cas proteins, e.g., Cas12, including without being limited thereto Cas 12a, Cas 12f, Cas12b, Cas12i, Cas12e and Cas12g; and type VI Cas proteins, e.g., Cas13, including without being limited thereto Cas13a, Cas13b, Cas13c and Cas13d, including engineered variants thereof (engineered Cas variants). In certain embodiments the Cas protein is selected from the group consisting of Cas9 protein, Cas 12 protein, Cas 13 protein, and engineered variants thereof, preferably the Cas protein is a Cas9 protein, a Cas 12 protein or an engineered variant thereof, more preferably the Cas protein is a Cas9 protein or an engineered variant thereof, such as a base editor or a prime editor.

Engineered Cas variants include, without being limited thereto, mutant and/or fusion proteins, such as variants with altered PAM compatibilities, such as less restrictive or different PAM compatibility of Cas9 or Cas12 variants; variants with higher DNA specificity, such as variants with reduced off-target Cas nuclease activity (e.g., eSpCas (1.1), SpCas9-HF1, HypaCas9, evoCas9, Sniper-Cas9, HiFiCas9, enAsCas12a-HF1); engineered Cas-domain-fused transposase and recombinase systems; base editors and prime editors.

Commonly used exemplary engineered variants are, for example, Cas nickase (e.g., nickase Cas9n) comprising an inactivated nuclease (e.g., HNH) and a nuclease-deficient dCas (e.g., dCas9), with impaired or no nuclease activity.

Cas proteins may be derived from different species, such as Streptococcus pyogenes, Staphylococcus aureus, Campylobacter jejuni, Streptococcus thermophilus or Neisseria meningitidis. For example, Cas9 orthologs include, without being limited thereto, Streptococcus pyogenes Cas9 (SpCas9), Staphylococcus aureus Cas9 (SaCas9), Campylobacter jejuni Cas9 (CjCas9), Streptococcus thermophilus Cas9 (StCas9), and Neisseria meningitidis Cas9 (NmeCas9) from. Cas orthologs may differ in the recognized PAM sequences and in size. The most often used Cas9 protein is SpCas9.

In certain embodiments the Cas protein is a Cas9, a Cas12, a Cas13 protein or an engineered variant thereof (also referred to as derivative thereof). In some embodiments the Cas protein is a base editor or a prime editor, preferably a Cas9 base editor or a Cas9 prime editor.

The Cas protein forms the Cas protein/gRNA complex, thus in certain embodiments the one or more Cas protein/gRNA RNP complex(es) is/are Cas9/gRNA RNP complex(es), preferably Cas9/sgRNA RNP complex(es), optionally further comprising a donor DNA. This includes Cas9 nucleases as well as fusion proteins thereof, such as base editors and prime editors.

The nanoparticle according to the invention comprises one or more Cas protein/gRNA RNP complex(es) and hence may contain more than one Cas protein/gRNA RNP complexes. In certain embodiments, the nanoparticle comprises two or more Cas protein/gRNA RNP complexes as cargo, preferably wherein the two or more Cas protein/gRNA RNP complexes comprise two or more different gRNAs targeting the same or different genes, preferably different genes. In a preferred embodiment the two or more gRNAs are two or more different sgRNAs targeting the same or different genes, preferably different genes. Typically, the two or more Cas protein/gRNA RNP complexes only differ in the gRNA (or sgRNA), while the Cas protein is the same. The nanoparticle comprising one or more Cas protein/gRNA RNP complex(es) as cargo may further comprise a donor DNA for HDR. Thus, in certain embodiments, the nanoparticle comprises one or more Cas protein/gRNA RNP complexes and a donor DNA. While the donor DNA may be co-delivered with the one or more Cas protein/gRNA RNP complex(es), it may also be delivered separately by non-viral or viral delivery, e.g., an adeno-associated viral vector, a lentiviral vector or an adenoviral vector. The T-shape lipo-oligomers having the formula C-Y₃-Gtt2-K (K-(OHSteA) 2)-Gtt2-Y₃-C or Y₃-X¹-K (K-(LinA) 2)-X¹-Y₃ (particularly Y₃-TFEIDAtp-K (K-(LinA) 2)-TFEIDAtp-Y₃) were found to be particularly efficient in inducing homology directed repair (HDR).

### Oligonucleotides as Cargo

It was surprisingly further found that the sequence-defined T-shape lipo-oligomer as described herein may be used as carrier for nucleic acid delivery, such as siRNA or phosphorodiamidate morpholino oligomers (PMO) delivery. The sequence-defined T-shape lipo-oligomer as carrier may form nanoparticles with the nucleic acid, such as siRNA or PMO, for delivery. Because PMOs are not charged, the sequence-defined T-shape lipo-oligomer as carrier are preferably covalently linked to the PMO, preferably to the 5′- or 3′-end of the PMO. Preferably, the sequence-defined T-shape lipo-oligomer as carrier covalently linked to the PMO comprises the sequence of formula I and/or the artificial amino acid is selected from Stp, chGtp, dGtp or TFEIDAtp. The fatty acid is preferably OleA or LinA.

Thus, in another aspect the invention relates to a nanoparticle for siRNA or phosphorodiamidate morpholino oligomers (PMO) (cargo) delivery comprising one or more siRNA(s) or PMO(s) as cargo; and a carrier comprising a sequence-defined T-shape lipo-oligomer comprising an artificial amino acid, wherein the artificial amino acid is an oligo(alkylamino) acid, and wherein the T-shape lipo-oligomer comprises the sequence of formula l:

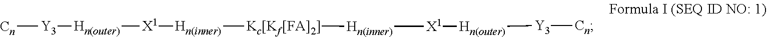

wherein
K_{c} is a central lysine;
Kᵣ is a further lysine linked to the ε-amino group of K_{c};
FA is a fatty acid covalently linked to the ε- and α-amino group of Kᵢ;
C is cysteine and n=0 or 1, preferably n=0
Y₃ is a tyrosine tripeptide;
H is histidine and n₍ₒᵤₜₑᵣ₎ and n₍ᵢₙₙₑᵣ₎ are independently selected from 0, 1 or 2; and
X¹ is an oligo(alkylamino) acid of formula IIa:

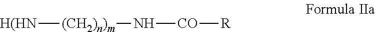

wherein n=2 or 3, alternating 2 and 3 or alternating 3 and 2; and m=3 or 4;
wherein R is
—(CH₂)_{y}—CO₂H, wherein y=2, 3 or 4;

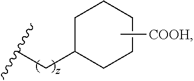

wherein z=0 or 1, preferably

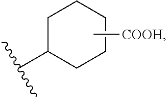

more preferably

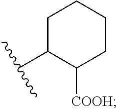 [SMILES: *C1CCCCC1C(=O)O]

—CH₂—CH(c-hexyl)-CH₂—CO₂H; or
—CH₂—NRₐ—CH₂—CO₂H, wherein Rₐ═H, CH₂—CFs, or
wherein the T-shape lipo-oligomer comprises the sequence of formula III:

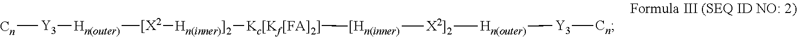

wherein
K_{c} is a central lysine;
Kᵣ is a further lysine linked to the ε-amino group of K_{c},
FA is a fatty acid covalently linked to the ε- and α-amino group of K_{f};
C is cysteine and n=0 or 1, preferably n=1
Y₃ is a tyrosine tripeptide;
H is histidine and n₍ₒᵤₜₑᵣ₎ and n₍ᵢₙₙₑᵣ₎ are independently selected from 0 or, 1; and
X² is an oligo(alkylamino) acid of formula IIa:

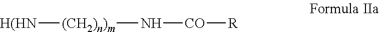

wherein n=2 or 3, alternating 2 and 3 or alternating 3 and 2; and m=3 or 4;
wherein R is
(CH₂)_{y}—CO₂H, wherein y=3 or 4;

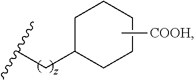

wherein z=0 or 1, preferably

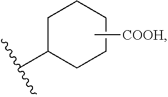

more preferably

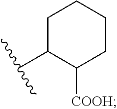 [SMILES: *C1CCCCC1C(=O)O]

—CH₂—CH(c-hexyl)-CH₂—CO₂H; or
—CH₂—NRₐ—CH₂—CO₂H, wherein Rₐ═CH₂—CF₃.

In a related aspect the invention relates to a nanoparticle for siRNA or phosphorodiamidate morpholino oligomers (PMO) (cargo) delivery comprising one or more siRNA(s) or PMO(s) as cargo; and a carrier comprising a sequence-defined T-shape lipo-oligomer comprising
(c) a headgroup comprising an oligomer comprising
   i. a central lysine (K_{c}), and
   ii. an N-terminal peptide chain and a symmetrical C-terminal peptide chain referred to the central lysine, each comprising an artificial amino acid, wherein the artificial amino acid is an oligo(alkylamino) acid; and
(d) a hydrophobic tail comprising
   i. two fatty acids (FA) covalently linked to the ε- and α-amino group of a further lysine (K_{f})
   ii. wherein Kᵣ is linked to the ε-amino group of the central lysine (K_{c});
wherein the T-shape lipo-oligomer comprises the sequence of formula I:

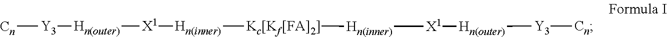

wherein
C is cysteine and n=0 or 1, preferably n=0
Y₃ is a tyrosine tripeptide;
H is histidine and n₍ₒᵤₜₑᵣ₎ and n₍ᵢₙₙₑᵣ₎ are independently selected from 0, 1 or 2; and
X¹ is an oligo(alkylamino) acid of formula IIIa:

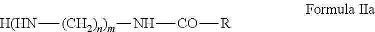

wherein n=2 or 3, alternating 2 and 3 or alternating 3 and 2; and m=3 or 4;
wherein R is
(CH₂)_{y}-CO₂H, wherein y=2, 3 or 4;

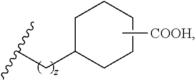

wherein z=0 or 1, preferably

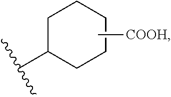

more preferably

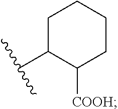 [SMILES: *C1CCCCC1C(=O)O]

—CH₂—CH(c-hexyl)-CH₂—CO₂H; or
—CH₂—NR—CH₂—CO₂H, wherein R═H, CH₂—CF₃; or
wherein the T-shape lipo-oligomer comprises the sequence of formula III:

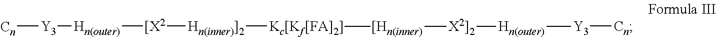

wherein
C is cysteine and n=0 or 1, preferably n=1
Y₃ is a tyrosine tripeptide;
H is histidine and n₍ₒᵤₜₑᵣ₎ and n₍ᵢₙₙₑᵣ₎ are independently selected from 0 or 1; and
X² is an oligo(alkylamino) acid of formula IIa:

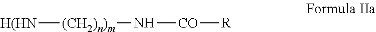

wherein n=2 or 3, alternating 2 and 3 or alternating 3 and 2; and m=3 or 4;
wherein R is
—(CH₂)_{y}—CO₂H, wherein y=3 or 4;

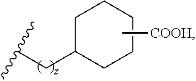

wherein z=0 or 1, preferably

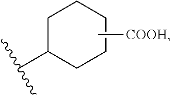

more preferably

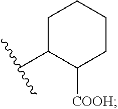 [SMILES: *C1CCCCC1C(=O)O]

—CH₂—CH(c-hexyl)-CH₂—CO₂H; or
—CH₂—NRₐ—CH₂—CO₂H, wherein Rₐ═CH₂—CF₃.

The embodiments as specified for the nanoparticle of the invention for Cas protein/gRNA ribonucleoprotein (RNP) complex delivery, particularly the embodiments further specifying the sequence-defined T-shape lipo-oligomer as carrier and the artificial amino acid similarly apply to nanoparticles for siRNA or PMO delivery.

For example, the two fatty acids are saturated or unsaturated C12 to C22 fatty acids, preferably the two fatty acids are selected from the group consisting of oleic acid (OleA), linoleic acid (LinA), linolenic acid (LenA) or hydroxy steric acid (OHSteA), preferably OleA, LinA, LenA or OHSteA, more preferably OleA, LinA, or OHSteA, or even more preferably LinA or OHSteA.

The sequence-defined T-shape lipo-oligomer as carrier forms nanoparticles with the negatively charged siRNA for delivery. Thus, in certain embodiments, the nanoparticle is for siRNA (cargo) delivery comprising one or more siRNA(s) as cargo; and a carrier comprising a sequence-defined T-shape lipo-oligomer comprising an artificial amino acid according to the invention and as specified herein.

Without being limited thereto, the siRNA and the sequence-defined T-shape lipo-oligomer are preferably mixed at a lipo-oligomer nitrogen (N) to nucleic acid phosphate (P) (siRNA) ratio (N/P ratio) of about 1:12 to 1:30, preferably of about 1:12 to 1:26. The person skilled in the art would know that only the protonatable nitrogens are considered for determining the N/P ratio. In an oligo(alkylamino) acid with five nitrogens, three are protonatable and with 4 nitrogens, two are protonatable.

The term “small interfering RNA” or “siRNA” refers to short single or double stranded ribonucleic acid molecules of typically 20-25 base pairs in length that bind to complementary single stranded ribonucleic acid molecules, such as messenger RNA (mRNA) and suppress their function, also referred to as RNA interference (RNAI). Thus, siRNA interferes with the expression of specific genes by degrading mRNA after transcription and preventing translation. Small interfering RNA (also referred to as short interfering RNA) are non-coding RNAs, i.e., not coding for a protein. Another RNA molecules operating within the RNAi pathway is microRNA (miRNA), which further has a similar length compared to siRNA.

In certain embodiments, the oligo(alkylamino) acid is a tetraethylenepentamine or a triethylenetetramine of formula IIb: H(HN—(CH₂)₂) m=3 or 4-NH—CO—R (Formula IIb); preferably a tetraethylenepentamine. Preferably, the T-shape lipo-oligomer comprises the sequence of formula I: Cₙ-Y₃-Hn₍ₒᵤₜₑᵣ₎-X¹-Hₙ₍ᵢₙₙₑᵣ₎-K₀[K[FA]-Hₙ₍ᵢₙₙₑᵣ₎—X¹-Hₙ₍ₒᵤₜₑᵣ₎-Y₃-Cₙ (Formula I); wherein C is cysteine and n=1; Y₃ is a tyrosine tripeptide; and H is histidine and n₍ₒᵤₜₑᵣ₎ and n₍ᵢₙₙₑᵣ₎ are 1.

According to formula I or III, cysteine (C) may be present (n=1) or absent (n=0) and is either present at the N-terminal and the C-terminal end or is absent from both ends. In a preferred embodiment the nanoparticles for siRNA delivery comprise one or more siRNA(s) as cargo and a carrier comprising a sequence-defined T-shape lipo-oligomer comprising the sequence of formula I or III, preferably of formula I, and the cysteine is present (n=1). This is in contrast to nanoparticles for Cas protein/gRNA ribonucleoprotein (RNP) delivery, where cysteine is not required. Cysteine may help to stabilize the nanoparticle by formation of disulfide bonds, which is beneficial for siRNA delivery as demonstrated in the examples. Stability may be further improved using artificial amino acids as described herein that are more hydrophobic than Stp. Preferably, the carrier comprising a sequence-defined T-shape lipo-oligomer comprising the sequence of formula I or III, preferably of formula I, further comprises histidine and n₍ᵢₙₙₑᵣ₎ and n₍ₒᵤₜₑᵣ₎ are 1. In certain embodiments the sequence-defined T-shape lipo-oligomer comprises a sequence of formula I selected from the group consisting of SEQ ID NOs: 7, 8, 9 and 10, preferably SEQ ID NO: 7.

The sequence-defined T-shape lipo-oligomer comprising the sequence of formula I or formula III as disclosed herein, preferably formula I, may further comprise a terminal functional group at the N-terminus or the C-terminus of the sequence-defined T-shape lipo-oligomer, wherein the functional group is an azido group, such as an azido-hexanoic acid or an azido-lysine. The functional group is, e.g., for coupling a targeting ligand. The functional group (azido group) may be N-terminally or C-terminally, preferably the functional group is N-terminally. Thus, the sequence-defined T-shape lipo-oligomer comprising the sequence of formula I or III may comprise a terminal azido group, such as an azido-hexanoic acid or an azido-lysine, preferably an azido-lysine. The azido group may be N-terminally or C-terminally, preferably the azido group is N-terminally. Although a single terminal azido group is sufficient, theoretically the sequence-defined T-shape lipo-oligomer may also comprise an azido group at the N-terminal and/or the C-terminal. In certain embodiments the azido-lysine (K (N3)) comprising sequence-defined T-shape lipo-oligomer comprises a sequence of formula I or III selected from the group consisting of SEQ ID NOs: 11-26 and 41-52, preferably 15, 17, 23 and 25, even more preferably 15.

PMOs are uncharged and it was therefore believed that they may not require carriers for cell entry. However, suitable carriers also improve PMO delivery. For efficient nanoparticle formation PMOs are preferably covalently linked to the carrier, i.e., the sequence-defined T-shape lipo-oligomer.

Thus, in certain embodiments the sequence-defined T-shape lipo-oligomer as carrier is covalently linked to the PMO, preferably to the 5′- or 3′-end of the PMO. The sequence-defined T-shape lipo-oligomer as carrier covalently linked to the PMO comprises the sequence of formula I or formula III, preferably the sequence of formula I. In a preferred embodiment the artificial amino acid is selected from Stp, Gtp, chGtp, dGtp, IDAtp or TFEIDAtp, more preferably Stp, chGtp, dGtp or TFEIDAtp.

Histidine containing sequence-defined T-shape lipo-oligomers as carrier generally mediated higher transfection efficiencies even at lower concentrations. The fatty acid is preferably OleA, LenA or LinA, more preferably OleA or LinA.

The term “phosphorodiamidate morpholino oligomer” as used herein is abbreviated as PMO and refers to antisense oligonucleotide (ASO) analogs that are artificial, uncharged antisense oligonucleotides with favorable stability, nuclease-resistance, low immunogenicity and toxicity. PMOs may be used to modulate gene expression by interfering with pre-mRNA splicing and are therefore a promising therapeutic molecule.

Without being limited thereto, the PMO and the sequence-defined T-shape lipo-oligomer are preferably conjugated at a molar ratio of PMO to sequence-defined T-shape lipo-oligomer (LP) of 1:3.

The resulting solution comprises PMO-LP conjugates and free LPs at a ratio 1:2, which forms the nanoparticles. Thus, in certain embodiments the nanoparticles comprise PMO-sequence-defined T-shape lipo-oligomer conjugates (PMO-LP) and un-conjugated sequence-defined T-shape lipo-oligomer (LP), preferably at a ratio 1:2. The terms “conjugated” and “coupled” are used synonymously herein mean covalently linked and hence also “conjugates” are covalently linked.

Thus, in another aspect the invention also relates to a conjugate comprising a phosphorodiamidate morpholino oligomer (PMO) covalently linked to a carrier comprising a sequence-defined T-shape lipo-oligomer, comprising an artificial amino acid, wherein the artificial amino acid is an oligo(alkylamino) acid, and wherein the T-shape lipo-oligomer comprises the sequence of formula I:

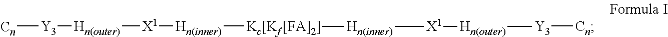

wherein
K_{c} is a central lysine;
K_{f} is a further lysine linked to the ε-amino group of K_{c};
FA is a fatty acid covalently linked to the ε- and amino group of Kᵢ,
C is cysteine and n=0 or 1, preferably n=0
Y₃ is a tyrosine tripeptide;
H is histidine and n₍ₒᵤₜₑᵣ₎ and n₍ᵢₙₙₑᵣ₎ are independently selected from 0, 1 or 2; and
X¹ is an oligo(alkylamino) acid of formula IIa:

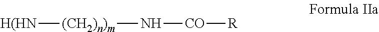

wherein n=2 or 3, alternating 2 and 3 or alternating 3 and 2; and m=3 or 4; and
wherein R is
—(CH₂)_{y}—CO₂H, wherein y=2, 3 or 4;

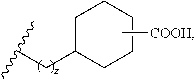

wherein z=0 or 1, preferably

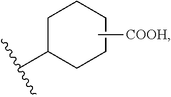

more preferably

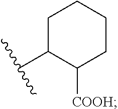 [SMILES: *C1CCCCC1C(=O)O]

—CH₂—CH(c-hexyl)-CH₂—CO₂H; or
—CH₂—NRₐ—CH₂—CO₂H, wherein Rₐ═H, CH₂—CFs, or
wherein the T-shape lipo-oligomer comprises the sequence of formula III:

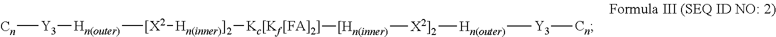

wherein
K_{c} is a central lysine;
K_{f} is a further lysine linked to the ε-amino group of K_{c}.
FA is a fatty acid covalently linked to the ε- and amino group of Kᵢ,
C is cysteine and n=0 or 1, preferably n=1
Y₃ is a tyrosine tripeptide;
H is histidine and n₍ₒᵤₜₑᵣ₎ and n₍ᵢₙₙₑᵣ₎ are independently selected from 0 or, 1; and
X² is an oligo(alkylamino) acid of formula IIIa:

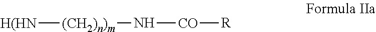

wherein n=2 or 3, alternating 2 and 3 or alternating 3 and 2; and m=3 or 4;
wherein R is
   —(CH₂)_{y}—CO₂H, wherein y=3 or 4;

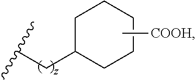

wherein z=0 or 1, preferably

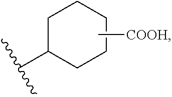

more preferably

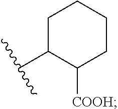 [SMILES: *C1CCCCC1C(=O)O]

—CH₂—CH(c-hexyl)-CH₂—CO₂H; or
—CH₂—NRₐ—CH₂—CO₂H, wherein Rₐ═CH₂—CF₃.
   and
wherein the two fatty acids are saturated or unsaturated C12 to C22 fatty acids, preferably the two fatty acids are selected from the group consisting of oleic acid (OleA), linoleic acid (LinA), linolenic acid (LenA) or hydroxy steric acid (OHSteA), preferably OleA, LinA or OHSteA, more preferably LinA or OHSteA.

The embodiments as specified for the nanoparticle of the invention for Cas protein/gRNA ribonucleoprotein (RNP) complex delivery, particularly the embodiments further specifying the sequence-defined T-shape lipo-oligomer as carrier and the artificial amino acid similarly apply to the conjugate comprising a phosphorodiamidate morpholino oligomer (PMO) covalently linked to a carrier comprising a sequence-defined T-shape lipo-oligomer.

In yet another aspect, the invention relates to a nanoparticle for PMO delivery comprising the conjugate comprising a phosphorodiamidate morpholino oligomer (PMO) covalently linked to a carrier comprising a sequence-defined T-shape lipo-oligomer according to the invention. The nanoparticle may further comprise the carrier comprising the sequence-defined T-shape lipo-oligomer not covalently linked to the PMO (free sequence-defined T-shape lipo-oligomer). Typically, the free sequence-defined T-shape lipo-oligomer and the covalently linked sequence-defined T-shape lipo-oligomer are the same, wherein the free sequence-defined T-shape lipo-oligomer may be additionally coupled to a targeting ligand.

In certain embodiments of the conjugate or the nanoparticle for PMO delivery, the oligo(alkylamino) acid is a tetraethylenepentamine or a triethylenetetramine of formula IIb: H(HN—(CH₂)₂)ₘ=3 or ₄—NH—CO—R (Formula IIb); preferably a tetraethylenepentamine. Preferably, the T-shape lipo-oligomer comprises the sequence of formula I: Cₙ—Y₃—Hn₍ₒᵤₜₑᵣ₎—X¹—Hₙ₍ᵢₙₙₑᵣ₎-K_{c} [K_{f} [FA]₂]-Hₙ₍ᵢₙₙₑᵣ₎-X¹-Hₙ₍ₒᵤₜₑᵣ₎-Y₃-Cₙ (Formula I); wherein C is cysteine and n=0 or 1, preferably 0; Y₃ is a tyrosine tripeptide; and H is histidine and n₍ₒᵤₜₑᵣ₎ and n₍ᵢₙₙₑᵣ₎ are 0 or 1, preferably 1.

According to formula I or III cysteine (C) may be present (n=1) or absent (n=0) and is either present at the N-terminal and the C-terminal end or is absent from both ends. In a preferred embodiment the nanoparticles for PMO delivery comprising one or more PMO as cargo and a carrier comprising a sequence-defined T-shape lipo-oligomer comprising the sequence of formula I or III, preferably of formula I, the cysteine is absent (n=0) and/or histidine is present, and n₍ᵢₙₙₑᵣ₎ and n₍ₒᵤₜₑᵣ₎ are 1. In certain embodiments the sequence-defined T-shape lipo-oligomer comprises a sequence of formula I selected from the group consisting of SEQ ID NOs: 7, 8, 9 and 10, preferably SEQ ID NO: 8.

The sequence-defined T-shape lipo-oligomer comprising the sequence of formula I or formula III as disclosed herein, preferably formula I, may further comprise a terminal functional group at the N-terminus or the C-terminus of the sequence-defined T-shape lipo-oligomer, wherein the functional group is (i) an azido group, such as an azido-hexanoic acid or an azido-lysine, or (ii) a thiol group provided that the sequence of formula I or III does not comprise a cysteine. Preferably, the functional group is an azido group. The functional group is used for coupling the PMO as cargo. The functional group (azido group or thiol group) may be N-terminally or C-terminally, preferably the functional group is N-terminally. Thus, the sequence-defined T-shape lipo-oligomer comprising the sequence of formula I or III may comprise a terminal azido group, such as an azido-hexanoic acid or an azido-lysine, preferably an azido-lysine. The azido group may be N-terminally or C-terminally, preferably the azido group is N-terminally. Although a single terminal azido group is sufficient, theoretically the sequence-defined T-shape lipo-oligomer may also comprise an azido group at the N-terminal and/or the C-terminal. In certain embodiments the azido-lysine (K (Ns)) comprising sequence-defined T-shape lipo-oligomer comprises a sequence of formula I or III selected from the group consisting of SEQ ID NOs: 11-26 and 41-52, preferably SEQ ID NOs: 15, 16, 17, 18, 23, 24, 25 and 26, more preferably SEQ ID NO: 16. The functional group may further be used for coupling a targeting ligand. Since not all LPs are covalently linked to the PMO, others may be conjugated with the targeting ligand.

A terminal azido group or a single terminal thiol group allows coupling of the PMO (and optionally the targeting ligand) using click chemistry or using thiol conjugation, respectively. Preferably, the PMO as cargo is coupled to the azido-group via click chemistry, more preferably click chemistry with a dibenzocylooctyne-coupled PMO. Click chemistry is a general term for highly specific, in many cases biorthogonal, covalent conjugation reactions, that are modular, efficient and relatively insensitive to solvent parameters, water and oxygen. Various click chemistry reactions are known in the art and the person skilled in the art would know how to select a specific click chemistry reaction for a certain conjugation and particular for a certain protein or oligomer, such as the sequence-defined T-shape lipo-oligomer. Typical click reactions, without being limited thereto, are copper-catalyzed azide-alkylene cycloaddition (CuAAC) (copper-catalyzed reaction of an azide with an alkyne), copper-free azide-alkyne cycloaddition, such as strain-promoted azide-alkyne cycloaddition (SPAAC), Diels-Alder or inverse electron Diels-Alder reaction, and alkene-tetrazole photoclick reaction.

### Therapeutic and Non-Therapeutic Uses

Cas proteins are RNA-directed nucleases, wherein a small guide RNA (gRNA) complementary to a target site guides the nuclease to this site. For example, Cas9 or Cas12 introduces double-strand breaks (DSB) at targeted DNA loci (Cas9 blunt end, Cas12 staggered ends). Since this system is modular it can be readily engineered. One of two cellular mechanisms then repairs the cut DNA by non-homologous end-joining (NHEJ), which introduces insertions or deletions (indels) or by homology-directed repair (HDR), which uses a donor DNA as template to introduce specific modifications near the target site. Thus, for some applications delivery of just the DNA nuclease is sufficient. For example, the Cas protein/gRNA RNP complex can be used to knockout alleles that underlie autosomal dominant genetic disorders, such as Huntington's disease and amyotrophic lateral sclerosis or for exon skipping or removal of a cryptic splice site, such as for Duchenne's muscular dystrophy and Leber's congenital amaurosis type 10, respectively. Moreover, base editors may be used to edit point-mutations in disease-causing alleles and the more recently developed prime editors may be used to correct not only point mutations, but also small indels without the induction of a double-stranded break. In addition to base editors and prime editors genome editing may be effected using nuclease-mediated, double-stranded break to trigger HDR via the co-delivery of a donor DNA.

In one aspect, the nanoparticle of the invention is used in therapy. Since the Cas proteins are so versatile a large variety of diseases can be treated, including, without being limited thereto, a disease selected from the group consisting of cancer, a genetic disease, an infectious disease, a cardiovascular disease, a metabolic disease, a neurodegenerative or neuromuscular disease, a hematological disease, a hereditary eye disease and an autoimmune disease. Similarly, siRNA and PMOs are versatile and can be used in treating a large variety of diseases, including, without being limited thereto, a disease selected from the group consisting of cancer, a genetic disease, an infectious disease, a cardiovascular disease, a metabolic disease, a neurodegenerative or neuromuscular disease, a hematological disease, a hereditary eye disease and an autoimmune disease.

In another aspect, the nanoparticle of the invention is used in treating cancer, a genetic disease, an infectious disease, a cardiovascular disease, a metabolic disease, a neurodegenerative or neuromuscular disease, a hematological disease, a hereditary eye disease or an autoimmune disease.

The nanoparticle according to the invention may be used in vivo or ex vivo for Cas protein/gRNA RNP, siRNA or PMO delivery. Thus, the nanoparticle for use in therapy is delivered to a cell of the subject to be treated in vivo or ex vivo. For therapeutic uses ex vivo, nanoparticle delivery to cells of the subject to be treated is followed by adoptive cell transfer of said cells to said subject. For Cas protein/gRNA RNP delivery mediating homology directed repair nanoparticles comprising the T-shape lipo-oligomers having the formula C-Y₃-Gtt₂-K(K-(OHSteA) 2)-Gtt₂-Y₃-C or Y₃-X¹-K (K-(LinA) 2)-X¹-Y₃ (particularly Y₃-TFEIDAtp-K (K-(LinA) 2)-TFEIDAtp-Y₃) may be particularly suitable.

In vivo delivery involves local administration or systemic administration and may require the coupling of a targeting ligand. Alternatively, targeted delivery may be achieved for cell types such as macrophages, with a high degree of phagocytosis and endocytosis. The nanoparticle may therefore be administered by any route, including, without being limited thereto intravenous administration, subcutaneous administration, intradermal administration, intramuscular administration oral administration, intranasal administration, vaginal administration, intravitreal administration, or intrathecal administration.

Delivery may also be ex vivo to a cell of the subject to be treated followed by adoptive cell transfer. Prior to adoptive cell transfer the cell may be screened for successful genome editing mediated by the Cas protein/gRNA RNP. Alternatively, prior to adoptive cell transfer the cell may be screened for successful gene silencing by siRNA or successful gene modulation by PMOs. Routes for adoptive transfer of the genome edited (or siRNA or PMO modified) cells to the subject are known in the art and include, without being limited thereto, intravenous administration, subcutaneous administration and intramuscular administration, particularly intravenous administration.

In yet another aspect the invention relates to a pharmaceutical composition comprising a nanoparticle and at least one pharmaceutically acceptable excipient. The pharmaceutical composition comprising the nanoparticle according to the invention may be cryo-conserved, lyophilized or in an isotonic solution (i.e., in a physiological buffer).

In addition to in vivo or ex vivo delivery, the nanoparticle according to the invention may further be used for in vitro Cas protein/gRNA RNP delivery, siRNA delivery or PMO delivery, i.e., for non-therapeutic purposes.

Thus, in another aspect the invention relates to an in vitro method for transfecting mammalian cells with one or more Cas protein/gRNA complex (or one or more siRNA(s) or PMO(s) comprising contacting a mammalian cell in vitro with the nanoparticle according to the invention or the conjugate according to the invention. Any mammalian cell may be suitable in the context of the present invention including cell lines and primary cells, suspension and adherent cells or even organoids. For example, the mammalian cells may be human or rodent cells, including, without being limited thereto CHO cells

BHK cells, HEK293 cells, HeLa cells, HepG2 cells and derivatives thereof. The person skilled in the art will understand that derivatives of CHO cells include, e.g., glutathione deficient CHO cells such as CHO-K1 cells and the like. Moreover, derivatives of HEK 293 cells include, e.g., HEK293T, HEK293E, HEK293F, HEK293SF cells and the like.

In yet another aspect, the invention relates to a use of the sequence-defined T-shape lipo-oligomer (or a method comprising said use) for cellular delivery of a Cas protein/gRNA ribonucleoprotein (RNP) complex, an siRNA or a PMO, preferably a Cas protein/gRNA ribonucleoprotein (RNP) complex, into a target cell, wherein the sequence defined T-shape lipo-oligomer comprising the sequence of formula!:

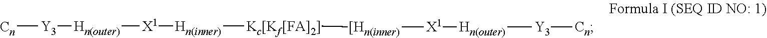

as disclosed and specified herein, or of formula III:

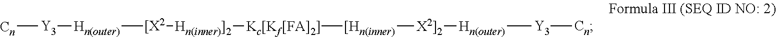

as disclosed and specified herein.

Particularly, wherein the T-shape lipo-oligomer comprises the sequence of formula I:

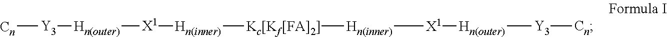

wherein
K_{c} is a central lysine;
K_{f} is a further lysine linked to the ε-amino group of K_{c};
FA is a fatty acid covalently linked to the ε- and α-amino group of K_{f},
C is cysteine and n=0 or 1, preferably n=0
Ys is a tyrosine tripeptide;
H is histidine and n₍ₒᵤₜₑᵣ₎ and n₍ᵢₙₙₑᵣ₎ are independently selected from 0, 1 or 2; and
X¹ is an oligo(alkylamino) acid of formula IIIa:

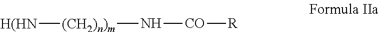

wherein n=2 or 3, alternating 2 and 3 or alternating 3 and 2; and m=3 or 4; and
wherein R is
(CH₂)_{y}—CO₂H, wherein y=2, 3 or 4;

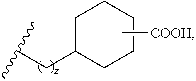

wherein z=0 or 1, preferably

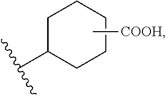

more preferably

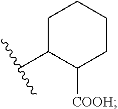 [SMILES: *C1CCCCC1C(=O)O]

—CH₂—CH(c-hexyl)-CH₂—CO₂H; or
—CH₂—NRₐ—CH₂—CO₂H, wherein Rₐ═H, CH₂—CF₃; or
wherein the T-shape lipo-oligomer comprises the sequence of formula III:

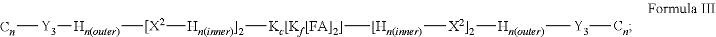

wherein
K_{c} is a central lysine;
Kᵣ is a further lysine linked to the ε-amino group of K_{c}.
FA is a fatty acid covalently linked to the ε- and α-amino group of Kᵣ,
C is cysteine and n=0 or 1, preferably n=1
Y₃ is a tyrosine tripeptide;
H is histidine and n₍ₒᵤₜₑᵣ₎ and n₍ᵢₙₙₑᵣ₎ are independently selected from 0 or 1; and
X² is an oligo(alkylamino) acid of formula IIa:

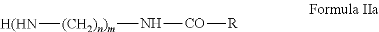

wherein n=2 or 3, alternating 2 and 3 or alternating 3 and 2; and m=3 or 4; and
wherein R is
—(CH₂)_{y}—CO₂H, wherein y=3 or 4;

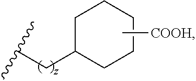

wherein z=0 or 1, preferably

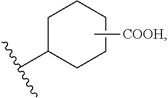

more preferably

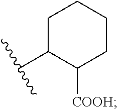 [SMILES: *C1CCCCC1C(=O)O]

—CH₂—CH(c-hexyl)-CH₂—CO₂H; or
—CH₂—NRₐ—CH₂—CO₂H, wherein Rₐ═CH₂—CF₃.

The embodiments and disclosures as specified for the nanoparticle of the invention similarly apply to this aspect. For mediating homology directed repair nanoparticles comprising the T-shape lipo-oligomers having the formula C-Y₃-Gtt₂-K(K-(OHSteA)₂)-Gtt₂-Y₃-C or Y₃-X¹-K (K-(LinA) 2)-X¹-Y₃ (particularly Y₃-TFEIDAtp-K(K-(LinA)₂)-TFEIDAtp-Y₃) may be particularly suitable.

In yet another aspect, the invention relates to a use of an artificial amino acid in a sequence-defined T-shape lipo-oligomer (or a method comprising said used) for cellular delivery of a Cas protein/gRNA ribonucleoprotein (RNP) complex, an siRNA or a PMO, preferably a Cas protein/gRNA ribonucleoprotein (RNP) complex into a target cell, wherein the artificial amino acid is an oligo(alkylamino) acid selected from the group consisting of Gtp, Gtt, Htp, Ptp, IDAtp, TFE-IDAtp, GEIPA, Ntp, and chGtp. In certain embodiments, the T-shape lipo-oligomer comprises the sequence of formula I:
Cₙ—Y₃-Hn₍ₒᵤₜₑᵣ₎-X¹-Hn (inner)-K-[K [FA] 2]-Hn (inner)-X¹-Hn₍ₒᵤₜₑᵣ₎-Y₃-Cₙ (Formula I; SEQ ID NO: 1), as disclosed and specified herein. In a preferred embodiment the oligo(alkylamino) acid (artificial amino acid) is selected from the group consisting of Gtp, Gtt, Htp, TFE-IDAtp, GEIPA, and chGtp, more preferably Htp, TFE-IDAtp, and chGtp. In certain other embodiments, the T-shape lipo-oligomer comprises the sequence of formula=disclosed and specified herein. In a preferred embodiment the oligo(alkylamino) acid (artificial amino acid) is selected from the group consisting of chGtp, Htp, TFE-IDAtp, Gtt or GEIPA. Preferably the oligo(alkylamino) acid is TFE-IDAtp or Gtt.

Preferably the uses of the invention are in vitro uses, i.e., for non-therapeutic purpose. In certain embodiments the target cell is a mammalian cell, preferably as specified herein above.

In yet another aspect, the invention relates to an artificial amino acid, wherein the artificial amino acid is an oligo(alkylamino) acid selected from the group consisting of the following formulas:

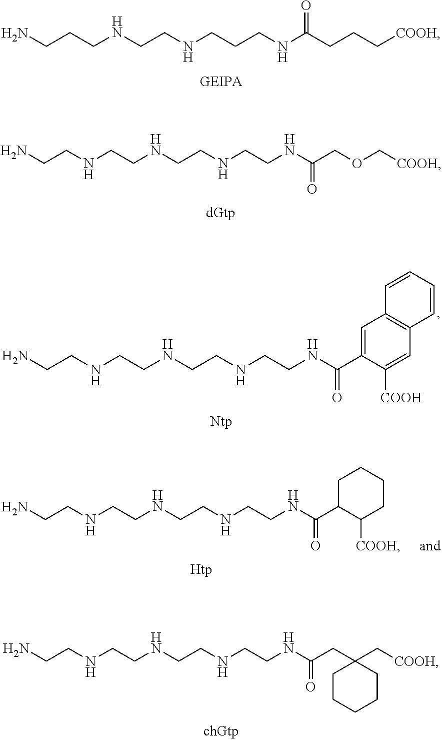 [SMILES: NCCCNCCNCCCNC(=O)CCCC(=O)O ; NCCNCCNCCNCCNC(=O)COCC(=O)O ; NCCNCCNCCNCCNC(=O)c1cc2ccccc2cc1C(=O)O ; NCCNCCNCCNCCNC(=O)C1CCCCC1C(=O)O ; NCCNCCNCCNCCNC(=O)CC1(CC(=O)O)CCCCC1]

in its protected and unprotected form.

In certain embodiments the use of the artificial amino acid according to the invention is for cellular delivery of a Cas protein/gRNA ribonucleoprotein (RNP) complex into a target cell, as specified herein above.

The oligo(alkylamino) acids are typically synthesized as protected building blocks prior to generating the sequence-defined lipo-oligomers, including the sequence-defined T-shape lipo-oligomers according to the invention. The person skilled in the art would know suitable protecting groups, such as fluorenylmethoxycarbonyl (Fmoc) and tert-butyloxycarbonyl (Boc). Building blocks with suitable amine protecting groups are known in the art and include without being limited thereto fluorenylmethoxycarbonyl (Fmoc), tert-butyloxycarbonyl (Boc), allyloxycarbonyl (Alloc), 1 methylethoxycarbonyl (Adpoc), 2,7-di-tert-buty [9-(10, 10-dioxo-10, 10, 10, 10-tetrahydrothioxanthyl)](Tmoc), benzyloxycarbonyl (Cbz, Z), 2-chlorobenzyloxycarbonyl(2-CI-Z), benzyl (Bn), triphenylmethyl (Trityl), 4-Methyltrityl (Mtt), 1-(4,4-dimethyl-2,6-dioxocyclohex-1-ylidene)ethyl (Dde), 1-(4,4-dimethyl-2,6-dioxocyclohex-1-ylidene) isovaleryl (ivDde), 2,7-disulfo-9-fluorenylmethoxycarbonyl (Smoc), trifluoroacetamide (TFA), tolylsuflonyl (Tos), 2-nitrobenzenesulfonyl (Ns). Preferred protecting groups are Fmoc at a terminal primary amine and Boc at internal secondary amines. Thus, the oligo(alkylamino) acids in their protected form may be Fmoc-GEIPA (Bocz)-OH, Fmoc-dGtp (Boca)-OH, Fmoc-Ntp (Bocs)-OH, Fmoc-Htp (Boc3)-OH and Fmoc-chGtp (Bocs)-OH:

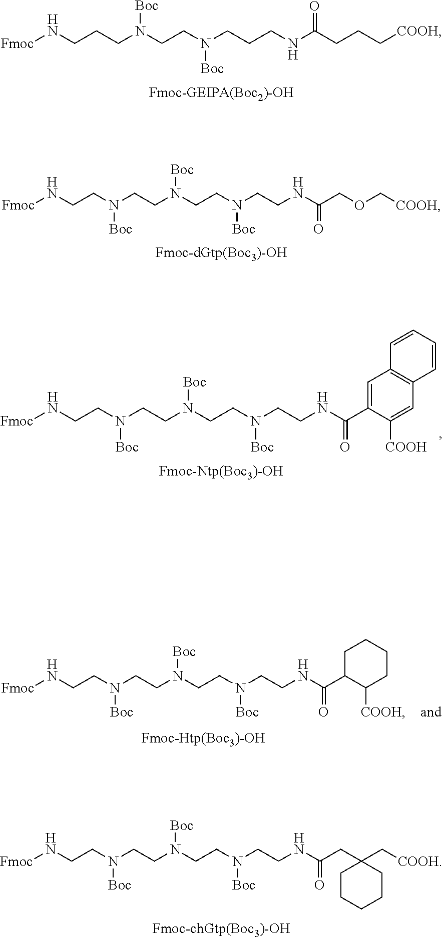

### EXAMPLES

### Synthesis of N-(tert-Butoxycarbonyl) iminodiacetic acid (Boc-IDA)

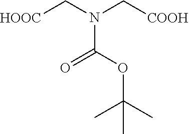 [SMILES: CC(C)(C)OC(=O)N(CC(=O)O)CC(=O)O]

N-tert-Butoxycarbonyl protected iminodiacetic acid (Boc-IDA) was synthesized using the procedure reported by Cookson et al. (Cookson, J., Amide functionalised dithiocarbamate ruthenium (II) bis-bipyridyl receptors: A new class of redox-responsive anion sensor, Inorganica Chimica Acta, 2008, 361, 1689-1698) with slight modification. Briefly 13.3 g iminodiacetic acid (0.1 mol) and 200 mL 1,4-dioxane were put into a 1 L round-bottom flask. 200 mL of 1 M sodium hydroxide solution were added and the mixture was stirred until a clear solution formed. 24.0 g of di-tert-butyl dicarbonate (0.11 mol, 1.1 eq) were dissolved in 50 mL 1,4-dioxane and added to the reaction mixture, which was stirred at RT for 72 h. The mixture was concentrated to approx. 200 mL under reduced pressure, washed twice with 150 ml diethyl ether. After acidification with 100 mL 10% HCl, the reaction product was extracted with EtOAc (3×150 mL). The organic phases were combined and dried over anhydrous sodium sulfate. The EtOAc was evaporated after filtration, yielding 22.3 g of Boc-IDA (0.096 mol, 96%) as white crystals.

### Synthesis of N-(tert-Butoxycarbonyl) iminodiacetic acid anhydride (Boc-IDA anhydride)

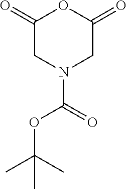

The cyclic anhydride of Boc-IDA was prepared by using dicyclohexylcarbodiimide (DCC) as dehydrating agent. 10.0 g of Boc-IDA (43 mmol) were put into a 500 mL round-bottom flask and 250 mL DCM were added. 8.9 g of DCC (43 mmol, 1 eq) were dissolved in 50 mL DCM and added into the round-bottom flask. The heterogeneous mixture was stirred at RT overnight. The next day the mixture was concentrated to a volume of approx. 100 mL under reduced pressure and the insoluble dicyclohexyl urea was removed by filtration. The DCM was removed in the rotary evaporator and at high vacuum to yield 8.4 g of Boc-IDA anhydride (39 mmol, 91%) as a solid.

### Synthesis of N-(Trifluoroethyl) iminodiacetic acid (TFE-IDA)

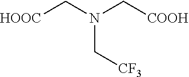 [SMILES: O=C(O)CN(CC(=O)O)CC(F)(F)F]

TFE-IDA was prepared by modification of the protocol for the synthesis of methyliminodiacetic acid published by Berchet (Berchet, G. J., METHYLIMINODIACETIC ACID, Organic Syntheses, 18 (1938) 56). 27.9 g chloroacetic acid (295.2 mmol, 2 eq) were placed in a 500 mL round-bottom flask. 22 mL H₂O were added and the flask was cooled in an ice bath. A cold solution of 29.5 g NaOH (738 mmol, 5 eq) in 93 mL H₂O was added slowly under stirring. After the complete addition, the cooling bath was removed and a solution of 20.0 g trifluoroethylamine hydrochloride (147.6 mmol, 1 eq) in 65 mL H₂O was added dropwise. After complete addition, the solution was stirred overnight. 60 g BaCl₂ dihydrate in approx. 140 mL boiling H₂O were added and the mixture was heated for 1.5 hours. The solid was filtered off and dried in the drying cabinet over 2 days yielding 27.6 g of TFE-IDA barium salt (78.8 mmol). The dry TFE-IDA barium salt was placed in a 500 mL round-bottom flask. 65 mL H₂O were added and heated to boiling. 31.5 mL of a 2.5 M H₂SO₄ solution (78.8 mmol) were added gradually over 1 hour under continuous heating to boiling. After cooling to RT, the mixture was centrifuged to remove the solid BaSO₄. The clear supernatant was freeze-dried yielding 16.1 g of TFE-IDA (74.8 mmol, 51%) as colorless crystals.

### Synthesis of N-(Trifluoroethyl) iminodiacetic acid anhydride (TFE-IDA anhydride)

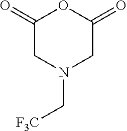 [SMILES: O=C1CN(CC(F)(F)F)CC(=O)O1]

The cyclic anhydride of TFE-IDA was prepared analogously to the anhydride of Boc-IDA by using DCC. 5.0 g TFE-IDA (23.2 mmol) were placed in a 250 mL round-bottom flask. 140 mL DCM were added. 4.8 g DCC (23.2 mmol, 1 eq) were dissolved in 30 mL DCM and added into the flask. The mixture was stirred at RT overnight. The mixture was concentrated to approx. 80 mL under reduced pressure and the insoluble dicyclohexyl urea was removed by filtration. The DCM was removed in the rotary evaporator and at high vacuum to yield 4.3 g TFE-IDA anhydride (21.8 mmol, 94%) as a solid.

### Synthesis of Protected Artificial Amino Acid Building Blocks (Depicted in Scheme 1)

The synthesis of protected building blocks Fmoc-Gtt (Boc₂)-OH, Fmoc-Stp (Bocs)-OH, Fmoc-Gtp (Bocs)-OH, Fmoc-Ptp (Bocs)-OH, Fmoc-Htp (Boc₃)-OH, Fmoc-chGtp (Boc₃)-OH, Fmoc-Ntp (Bocs)-OH, Fmoc-Boc-IDAtp (Bocs)-OH, Fmoc-TFE-IDAtp (Bocs)-OH and Fmoc-dGtp (Boc₃)-OH was carried out with cyclic dicarboxylic acid anhydrides analog to the description published by Schaffert et al. (Schaffert, D. et al., Novel Fmoc-polyamino acids for solid-phase synthesis of defined polyamidoamines, Organic letters, 2011, 13.7:1586-1589). Briefly, the two primary amines of the polyamines 1 (TETA, TEPA) were selectively protected with ethyl trifluoroacetate (EtOTFA). Subsequently the remaining secondary amines were Boc-protected by reaction with di-tert-butyl dicarbonate (Boc₂O) in a one-pot reaction. Work-up and recrystallization gave the compounds 2, bis-tfa-Tt (Boc₂) or bis-tfa-Tp (Bocs), respectively. The primary amines were deprotected by alkaline hydrolysis with aqueous NaOH containing 45% EtOH to obtain the compounds 3, Tt (Bocz) or Tp (Boc₃).

In the final step, the two primary amines were asymmetrically substituted by reaction with a cyclic anhydride (succinic anhydride for Fmoc-Stp (Boc₃)-OH; glutaric anhydride for Fmoc-Gtp (Bocs)-OH and Fmoc-Gtt (Bocz)-OH; phthalic anhydride for Fmoc-Ptp (Bocs)-OH; 3-oxaspiro[5,5]undecan-2,4-dion for Fmoc-chGtp (Bocs)-OH; 1,2-cyclohexanedicarboxylic anhydride for Fmoc-Htp (Bocs)-OH; 2,3-napthalic anhydride for Fmoc-Ntp (Bocs)-OH; diglycolic anhydride for Fmoc-dGtp (Bocs)-OH) and N-(fluorenyl-9-methoxycarbonyloxy)-succinimid (Fmoc-OSu). Purification of the products 4a was carried out by dry column vacuum chromatography (DCVC) (D. S. Pedersen, C. Rosenbohm, Dry Column Vacuum Chromatography Synthesis, (2001) 2431-2434).

In case of the IDA derived building blocks for the production of Fmoc-Boc-IDAtp (Boc₃)-OH and Fmoc-TFE-IDAtp (Bocs)-OH, TEPA (n=3) was used as the polyamine element. The first steps to obtain Tp (Bocs) were carried out in the same manner as described above for the other building blocks. In the last step, the custom-synthesized IDA derived anhydrides (Boc-IDA and TFE-IDA anhydride, respectively) were used and the synthesis protocol described above was adapted without additional modification.

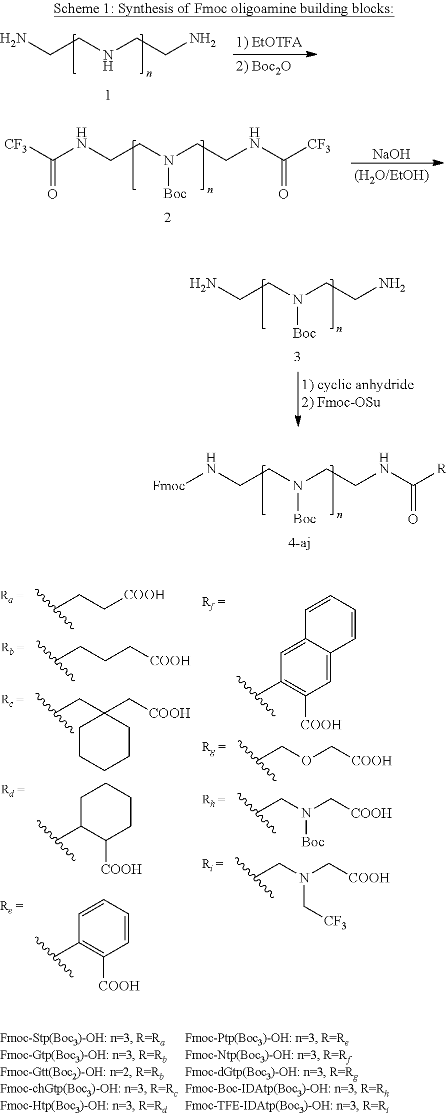

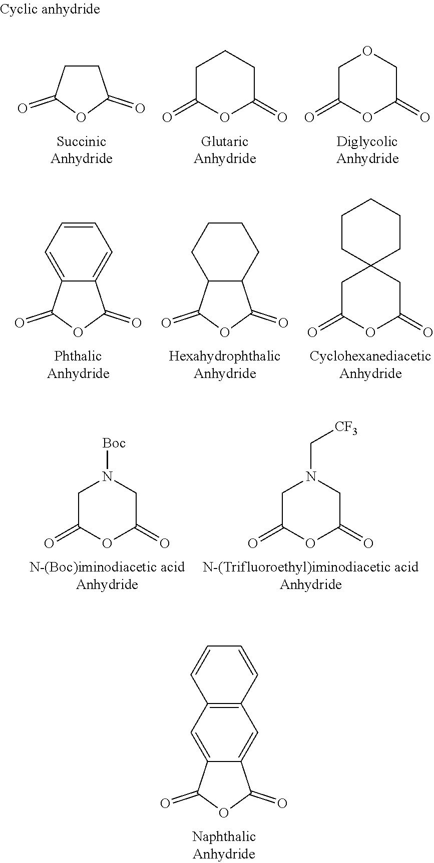

### Synthesis of Protected Artificial Amino Acid Building Block Fmoc-GEIPA (Boc₂)-OH (Depicted in Scheme 2)

The protected building block Fmoc-GEIPA (Boc₂)-OH was synthesized by exact adaption of the synthesis protocol described above and using the oligoamine 3,3′-ethylenediiminodipropylamine instead of TETA or TEPA.

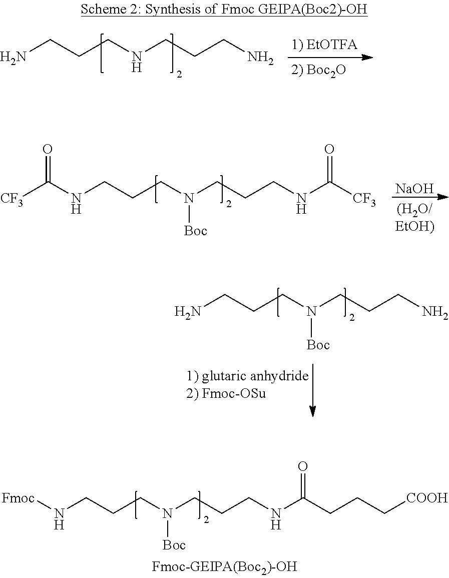

Analytical results:

Fmoc-Stp (Boc₃)-OH. Yield: 77%, ¹H NMR (400 MHZ, CDCl₃) δ (ppm)=7.78 (d, 2H, ArH-Fmoc), 7.61 (d, 2H, ArH-Fmoc), 7.41 (t, 2H, ArH-Fmoc), 7.31 (t, 2H, ArH-Fmoc), 4.40 (m, 2H, CH₂-Fmoc), 4.21 (m, 1H, CH-Fmoc), 3.47-3.20 (m, 16H, CH₂-Tepa), 2.67 (t, 2H, CH₂CONH-Suc), 2.48 (m, 2H, CH₂COOH-Suc), 1.46 (s, 27H, CH₃-tert-but). ESI-MS: calculated 811.4; found 810.4 [M−H]⁻.

Fmoc-Boc-IDAtp (Boc₃)-OH. Yield: 29%, ¹H NMR (400 MHz, CDCl) δ (ppm)=7.77 (d, 2H, ArH-Fmoc), 7.59 (d, 2H, ArH-Fmoc), 7.40 (t, 2H, ArH-Fmoc), 7.31 (t, 2H, ArH-Fmoc), 4.40 (m, 2H, CH₂-Fmoc), 4.20 (m, 1H, CH-Fmoc), 3.91 (s, 4H, CH₂-IDA) 3.48-3.19 (m, 16H, CH₂-Tepa), 1.43 (s, 36H, CH₃-tert-but). ESI-MS: calculated 926.5; found 925.5 [M−H].

Fmoc-dGtp (Bocs)-OH. Yield: 73%, ¹H NMR (400 MHZ, CDCl₃) Ö (ppm)=7.77 (d, 2H, ArH-Fmoc), 7.60 (d, 2H, ArH-Fmoc), 7.42 (t, 2H, ArH-Fmoc), 7.32 (t, 2H, ArH-Fmoc), 4.41 (m, 2H, CH₂-Fmoc), 4.23 (m, 1H, CH-Fmoc), 4.17 (d, 2H, CH₂CONH-diglycolic acid), 4.10 (d, 2H, CH₂COOH-diglycolic acid), 1.45 (s, 27H, CH₃-tert-but). ESI-MS: calculated 827.4; found 826.4 [M−H].

Fmoc-Gtp (Boc₃)-OH. Yield: 68%, ¹H NMR (400 MHZ, CDCl₃) õ (ppm)=7.79 (d, 2H, ArH-Fmoc), 7.60 (d, 2H, ArH-Fmoc), 7.41 (t, 2H, ArH-Fmoc), 7.32 (t, 2H, ArH-Fmoc), 4.41 (m, 2H, CH₂-Fmoc), 4.22 (m, 1H, CH-Fmoc), 3.48-3.24 (m, 16H, CH₂-Tepa), 2.42 (t, 2H, CH₂CONH-Glu), 2.29 (t, 2H, CH₂COOH-Glu), 1.97 (m, 2H, CH₂CH₂CH₂COOH-Glu), 1.47 (s, 27H, CH₃-tert-but). ESI-MS: calculated 825.4; found 824.4 [M−H]⁻.

Fmoc-Htp (Boc₃)-OH. Yield: 55%, ¹H NMR (400 MHZ, CDCl₃) õ (ppm)=7.69 (d, 2H, ArH-Fmoc), 7.51 (d, 2H, ArH-Fmoc), 7.32 (t, 2H, ArH-Fmoc), 7.23 (t, 2H, ArH-Fmoc), 4.30 (m, 2H, CH₂-Fmoc), 4.12 (m, 1H, CH-Fmoc), 3.48-3.08 (m, 16H, CH₂-Tepa), 2.50 (d, 1H, CHCONH-Hexahydrophthalic acid), 2.16 (s, 1H, CHCOOH-Hexahydrophthalic acid), 1.89-1.24 (m, 35H, CH₂-Hexahydrophthalic acid, CHa-tert-but). ESI-MS: calculated 865.5; found 864.5 [M−H]-Fmoc-chGtp (Boc₃)-OH. Yield: 63%, ¹H NMR (400 MHZ, CDCl) õ (ppm)=7.69 (d, 2H, ArH-Fmoc), 7.51 (d, 2H, ArH-Fmoc), 7.32 (t, 2H, ArH-Fmoc), 7.23 (t, 2H, ArH-Fmoc), 4.30 (m, 2H, CH₂-Fmoc), 4.12 (m, 1H, CH-Fmoc), 3.48-3.08 (m, 16H, CH₂-Tepa), 2.50 (d, 1H, CHCONH-Hexahydrophthalic acid), 2.16 (s, 1H, CHCOOH-Hexahydrophthalic acid), 1.89-1.24 (m, 35H, CH₂-Hexahydrophthalic acid, CH₃-tert-but). ESI-MS: calculated 893.5; found 892.5 [M−H]⁻.

Fmoc-TFE-IDAtp (Boc₃)-OH. Yield: 28%, ¹H NMR (400 MHZ, CDCl₃) δ (ppm)=7.69 (d, 2H, ArH-Fmoc), 7.51 (d, 2H, ArH-Fmoc), 7.32 (t, 2H, ArH-Fmoc), 7.23 (t, 2H, ArH-Fmoc), 4.30 (m, 2H, CH₂-Fmoc), 4.12 (m, 1H, CH-Fmoc), 3.48-3.08 (m, 16H, CH₂-Tepa), 2.50 (d, 1H, CHCONH-Hexahydrophthalic acid), 2.16 (s, 1H, CHCOOH-Hexahydrophthalic acid), 1.89-1.24 (m, 35H, CH₂-Hexahydrophthalic acid, CH₃-tert-but). ESI-MS: calculated 908.4; found 907.4 [M−H].

Fmoc-Gtt (Bocz)-OH. Yield: 66%, ¹H NMR (400 MHZ, CDCl₃) õ (ppm)=7.79 (d, 2H, ArH-Fmoc), 7.61 (d, 2H, ArH-Fmoc), 7.42 (t, 2H, ArH-Fmoc), 7.32 (t, 2H, ArH-Fmoc), 4.42 (m, 2H, CH₂-Fmoc), 4.22 (m, 1H, CH-Fmoc), 3.52-3.21 (m, 12H, CH₂-Teta), 2.41 (t, 2H, CH₂CONH-Glu), 2.28 (t, 2H, CH₂COOH-Glu), 1.98 (m, 2H, CH₂CH₂CH₂COOH-Glu), 1.45 (s, 18H, CHa-tert-but). ESI-MS: calculated 682.4; found 681.4 [M−H].

Fmoc-GEIPA (Bocz)-OH. Yield: 72%, ¹H NMR (400 MHZ, CDCl₃) õ (ppm)=7.78 (d, 2H, ArH-Fmoc), 7.62 (d, 2H, ArH-Fmoc), 7.41 (t, 2H, ArH-Fmoc), 7.32 (t, 2H, ArH-Fmoc), 4.42 (m, 2H, CH₂-Fmoc), 4.23 (m, 1H, CH-Fmoc), 3.43-3.03 (m, 12H, CH₂CH₂CH₂-GEIPA, CH₂CH₂-GEIPA), 2.44 (t, 2H, CH₂CONH-Glu), 2.30 (t, 2H, CH₂COOH-Glu), 1.99 (m, 2H, CH₂CH₂CH₂COOH-Glu), 1.71 (m, 4H, CH₂CH₂CH₂-GEIPA), 1.47 (s, 18H, CH₃-tert-but). ESI-MS: calculated 710.4; found 709.4 [M−H]. Fmoc-Ptp (Boc₃)-OH. Yield: 45%, ¹H NMR (400 MHZ, CDCl) õ (ppm)=7.81-7.25 (m, 12H, ArH-Fmoc, ArH-Phthalic acid), 4.41 (m, 2H, CH₂-Fmoc), 4.22 (m, 1H, CH-Fmoc), 3.69-3.17 (m, 16H, CH₂-Tepa), 1.44 (s, 27H, CH₃-tert-but). ESI-MS: calculated 859.4; found 858.4 [M−H]. Fmoc-Ntp (Boc₃)-OH. Yield: 52%, ¹H NMR (400 MHz, CDCls) õ (ppm)=7.98-7.17 (m, 14H, ArH-Fmoc, ArH-Naphthalenedicarboxylic acid), 4.29 (m, 2H, CH₂-Fmoc), 4.12 (m, 1H, CH-Fmoc), 3.62-3.12 (m, 16H, CH₂-Tepa), 1.34 (s, 27H, CH₃-tert-but). ESI-MS: calculated 909.4; found 908.4 [M−H].

Hydroxystearic acid (OHSteA). Yield: 84%, ¹H NMR (400 MHZ, CDCl₃) ö (ppm)=3.57-3.48 (q, 1H, CH-OH), 2.35-2.21 (t, 2H, CH₂COOH), 1.63-1.49 (q, 2H, CH₂CH₂COOH), 1.44-1.11 (m, 26H, —CH₂—), 0.87-0.72 (t, 3H, CH₃). ESI-MS: calculated 300.3; found 299.3 [M−H].

Sequence-defined oligomers comprising artificial amino acids synthesized by solid phase synthesis

Sequence defined oligomers comprising oligoamino amides were synthesized by solid-phase synthesis (Schaffert, D. et al., Angewandte Chemie International Edition, 2011, 50, 8986-8989) as described previously (Reinhard, S., Optimized Solid-Phase-Assisted Synthesis of Oleic Acid Containing siRNA Nanocarriers, ChemMedChem, 2017, 12, 1464-1470; Kuhn, J. et al., Delivery of Cas9/sgRNA Ribonucleoprotein Complexes via Hydroxystearyl Oligoamino Amides, Bioconjugate Chemistry, 2020, 31, 729-742) with slight modification. The sequence defined oligomers generated and tested for Cas9 RNP delivery are shown in Table 1. All oligomers were synthesized manually on 2-chlorotrityl chloride resin using standard Fmoc-based solid-phase peptide synthesis conditions. For standard Fmoc-α-amino acids, Fmoc-Stp (Bocs)-OH, Fmoc-Boc-IDAtp (Bocs)-OH, Fmoc-dGtp (Boc₃)-OH, Fmoc-Gtp (Boc₃)-OH, Fmoc-TFE-IDAtp (Boc₃)-OH, Fmoc-Gtt (Bocz)-OH, or Fmoc-GEIPA (Bocz)-OH, the coupling step was performed using 4 eq Fmoc-amino acid, 4 eq HOBt, 4 eq PyBOP, and 8 eq DIPEA in DCM/DMF (1/1, 5 mL g-1 resin) for 75 min. For Fmoc-Htp (Bocs)-OH or Fmoc-chGtp (Bocs)-OH, the coupling step was performed using 4 eq Fmoc-amino acid, 4 eq HOBt, 4 eq PyBOP, and 8 eq DIPEA in DCM/DMF (1/1, 5 mL g⁻¹ resin) containing 1% Triton X-100 overnight. For Fmoc-Ptp (Bocs)-OH or Fmoc-Ntp (Bocs)-OH, the coupling step was performed using 4 eq Fmoc-amino acid, 4 eq Oxyma, and 4 eq DIC in NMP (5 ml g-1 resin) overnight. Fmoc deprotection step was performed by 3 times incubation with 20% piperidine in DMF (5 mL g-1 resin) for 15 min. The resin was washed 3 times with DMF and 3 times with DCM after each coupling and deprotection step. A Kaiser test (Kaiser et al., Analytical Biochemistry, 34, 1970, 595-598.) was carried out after each coupling and deprotection step. In case of unexpected results (positive after coupling, negative after deprotection), the last coupling or deprotection step, respectively, was repeated. After the last deprotection of Fmoc-Lys (N3)-OH, the N-terminal amino group was protected with 10 eq BoczO and 10 eq DIPEA in DCM/DMF (1/1, 5 mL g-1 resin). Dde deprotection of the central Fmoc-Lys (Dde)-OH was accomplished by 15 times incubation with 2% hydrazine in DMF for 2 min. Subsequently, the resin was washed 5 times with DMF, 5 times with 10% DIPEA in DMF, and 3 times with DCM (5 mL g 1 resin each). Afterwards, Fmoc-Lys (Fmoc)-OH was introduced followed by the coupling of different fatty acids. The resin was washed 3 times with DMF and DCM and dried in vacuo. The oligomers were cleaved off the resin by incubation with pre-cooled cleavage cocktail TFA/EDT/H₂O/TIS (94/2.5/2.5/1, 10 mL g-1 resin) for 30 min. The cleavage solution was then immediately precipitated in pre-cooled MTBE/n-hexane (1/1, 40 mL). Afterwards, the oligomers were purified by size exclusion chromatography. The fractions were combined and snap frozen and lyophilized to obtain the final products.

TABLE 1
The sequence-defined T-shape lipo-oligomers synthesized and tested for Cas9 RNP
delivery

| Oligo | | SEQ ID | abbre- | | |
|---|---|---|---|---|---|
| ID | Sequence (N=>C)^{(a)(b)} | NO | viation | X | FA |
| 1208 | Y3-Stp2-K(K(OleA)2)-Stp2-Y3 | 62/107^{(b)} | Stp2 | Stp | OleA |
| 1209 | Y3-(H-Stp)2-H-K(K(OleA)2)-H-(Stp-H)2-Y3 | 60/105^{(b)} | Stp2-H | Stp | OleA |
| 1214 | C-Y3-(H-Stp)2-H-K(K(OleA)2)-H-(Stp-H)2- | 59/104^{(b)} | Stp2-HC | Stp | OleA |
| | Y3-C | | | | |
| 1278 | C-Y3-(H-Stp)2-H-K(K(SteA)2)-H-(Stp-H)2- | 59/104^{(b)} | Stp2-HC | Stp | SteA |
| | Y3-C | | | | |
| 1321 | Y3-(H-Stp)2-H-K(K(SteA)2)-H-(Stp-H)2-Y3 | 60/105^{(b)} | Stp2-H | Stp | SteA |
| 1337 | C-Y3-Stp2-K(K(SteA)2)-Stp2-Y3-C | 61/106^{(b)} | Stp2-C | Stp | SteA |
| 1338 | C-Y3-Stp2-K(K(OleA)2)-Stp2-Y3-C | 61/106^{(b)} | Stp2-C | Stp | OleA |
| 1340 | C-Y3-Stp2-K(K(CholA)2)-Stp2-Y3-C | 61/106^{(b)} | Stp2-C | Stp | CholA |
| 1361 | Y3-Stp2-K(K(SteA)2)-Stp2-Y3 | 62/107^{(b)} | Stp2-H | Stp | SteA |
| 1364 | Y3-Stp2-K(K(CholA)2)-Stp2-Y3 | 62/107^{(b)} | Stp2 | Stp | CholA |
| 1389 | C-Y3-H-(H-Stp)2-K(K(LinA)2)-(Stp-H)2-H- | 59/104^{(b)} | Stp2-HC | Stp | LinA |
| | Y3-C | | | | |
| 1390 | C-Y3-H-(H-Stp)2-K(K(LenA)2)-(Stp-H)2-H- | 59/104^{(b)} | Stp2-HC | Stp | LenA |
| | Y3-C | | | | |
| 1391 | Y3-Stp-K(K(OleA)2)-Stp-Y3 | 66/103^{(b)} | Stp1 | Stp | OleA |
| 1392 | Y3-Stp-K(K(LinA)2)-Stp-Y3 | 66/103^{(b)} | Stp1 | Stp | LinA |
| 1393 | Y3-Stp-K(K(LenA)2)-Stp-Y3 | 66/103^{(b)} | Stp1 | Stp | LenA |
| 1394 | Y3-Stp-K(K(CholA)2)-Stp-Y3 | 66/103^{(b)} | Stp1 | Stp | CholA |
| 1395 | Y3-H-Stp-H-K(K(OleA)2)-H-Stp-H-Y3 | 64/101^{(b)} | Stp1-H | Stp | OleA |
| 1396 | Y3-H-Stp-H-K(K(LinA)2)-H-Stp-H-Y3 | 64/101^{(b)} | Stp1-H | Stp | LinA |
| 1397 | Y3-H-Stp-H-K(K(LenA)2)-H-Stp-H-Y3 | 64/101^{(b)} | Stp1-H | Stp | LenA |
| 1398 | Y3-H-Stp-H-K(K(CholA)2)-H-Stp-H-Y3 | 64/101^{(b)} | Stp1-H | Stp | CholA |
| 1399 | Y3-Stp2-K(K(LinA)2)-Stp2-Y3 | 62/107^{(b)} | Stp2 | Stp | LinA |
| 1400 | Y3-Stp2-K(K(LenA)2)-Stp2-Y3 | 62/107^{(b)} | Stp2 | Stp | LenA |
| 1401 | Y3-H-(Stp-H)2-K(K(LinA)2)-(H-Stp)2-H-Y3 | 60/105^{(b)} | Stp2-H | Stp | LinA |
| 1402 | Y3-H-(Stp-H)2-K(K(LenA)2)-(H-Stp)2-H-Y3 | 60/105^{(b)} | Stp2-H | Stp | LenA |
| 1403 | Y3-H-(Stp-H)2-K(K(CholA)2)-(H-Stp)2-H-Y3 | 60/105^{(b)} | Stp2-H | Stp | CholA |
| 1445 | C-Y3-Stp2-K(K-(OHSteA)2)-Stp2-Y3-C | 61/106^{(b)} | Stp2-C | Stp | OHSteA |
| 1468 | Y3-Stp-K(K(SteA)2)-Stp-Y3 | 66/103^{(b)} | Stp1 | Stp | SteA |
| 1469 | Y3-Stp-K(K(OH-SteA)2)-Stp-Y3 | 66/103^{(b)} | Stp1 | Stp | SteA |
| 1470 | Y3-H-Stp-H-K(K(SteA)2)-H-Stp-H-Y3 | 64/101^{(b)} | Stp1-H | Stp | SteA |
| 1471 | Y3-H-Stp-H-K(K(OH-SteA)2)-H-Stp-H-Y3 | 64/101^{(b)} | Stp1-H | Stp | SteA |
| 1573 | Y3-Stp2-K(K(OHSteA)2)-Stp2-Y3 | 62/107^{(b)} | Stp2 | Stp | SteA |
| 1574 | Y3-(H-Stp)2-H-K(K(OHSteA)2)-H-(Stp-H)2- | 62/107^{(b)} | Stp2-H | Stp | SteA |
| | Y3 | | | | |
| 1575 | C-Y3-(H-Stp)2-H-K(K(CholA)2)-H-(Stp-H)2- | 59/104^{(b)} | Stp2-HC | Stp | CholA |
| | Y3-C | | | | |
| 1576 | C-Y3-(H-Stp)2-H-K(K(OHSteA)2)-H-(Stp- | 59/104^{(b)} | Stp2-HC | Stp | SteA |
| | H)2-Y3-C | | | | |
| 1577 | C-Y3-Stp-K(K(OleA)2)-Stp-Y3-C | 65/102^{(b)} | Stp1-C | Stp | OleA |
| 1578 | C-Y3-Stp-K(K(LinA)2)-Stp-Y3-C | 65/102^{(b)} | Stp1-C | Stp | LinA |
| 1579 | C-Y3-Stp-K(K(LenA)2)-Stp-Y3-C | 65/102^{(b)} | Stp1-C | Stp | LenA |
| 1580 | C-Y3-Stp-K(K(CholA)2)-Stp-Y3-C | 65/102^{(b)} | Stp1-C | Stp | CholA |
| 1581 | C-Y3-Stp-K(K(SteA)2)-Stp-Y3-C | 65/102^{(b)} | Stp1-C | Stp | SteA |
| 1582 | C-Y3-Stp-K(K(OHSteA)2)-Stp-Y3-C | 65/102^{(b)} | Stp1-C | Stp | OHSteA |
| 1583 | C-Y3-H-Stp-H-K(K(OleA)2)-H-Stp-H-Y3-C | 63/100^{(b)} | Stp1-HC | Stp | OleA |
| 1584 | C-Y3-H-Stp-H-K(K(LinA)2)-H-Stp-H-Y3-C | 63/100^{(b)} | Stp1-HC | Stp | LinA |
| 1585 | C-Y3-H-Stp-H-K(K(LenA)2)-H-Stp-H-Y3-C | 63/100^{(b)} | Stp1-HC | Stp | LenA |
| 1586 | C-Y3-H-Stp-H-K(K(CholA)2)-H-Stp-H-Y3-C | 63/100^{(b)} | Stp1-HC | Stp | CholA |
| 1587 | C-Y3-H-Stp-H-K(K(SteA)2)-H-Stp-H-Y3-C | 63/100^{(b)} | Stp1-HC | Stp | SteA |
| 1588 | C-Y3-H-Stp-H-K(K(OHSteA)2)-H-Stp-H-Y3- | 63/100^{(b)} | Stp1-HC | Stp | OHSteA |
| | C | | | | |
| 1589 | Y2-H-Stp-H-K(K(LinA)2)-H-Stp-H-Y2 | 67/141^{(b)} | Stp1-H | Stp | LinA |
| | | | (-Y1) | | |
| 1590 | Y-H-Stp-H-K(K(LinA)2)-H-Stp-H-Y | 68/142^{(b)} | Stp1-H | Stp | LinA |
| | | | (-Y2) | | |
| 1591 | H-Stp-H-K(K(LinA)2)-H-Stp-H | 69/143^{(b)} | Stp1-H | Stp | LinA |
| 1631 | C-Y3-Gtp2-K(K-(OHSteA)2)-Gtp2-Y3-C | 70/111^{(b)} | Gtp2-C | Gtp | OHSteA |
| 1633 | C-Y3-Htp2-K(K-(OHSteA)2)-Htp2-Y3-C | 71/112^{(b)} | Htp2-C | Htp | OHSteA |
| 1634 | C-Y3-Ptp2-K(K-(OHSteA)2)-Ptp2-Y3-C | 72/113^{(b)} | Ptp2-C | Ptp | OHSteA |
| 1635 | C-Y3-Ntp2-K(K-(OHSteA)2)-Ntp2-Y3-C | 73/114^{(b)} | Ntp2-C | Ntp | OHSteA |
| 1636 | C-Y3-Gtt2-K(K-(OHSteA)2)-Gtt2-Y3-C | 74/115^{(b)} | Gtt2-C | Gtt | OHSteA |
| 1637 | C-Y3-GEIPA2-K(K-(OHSteA)2)-GEIPA2- | 75/116^{(b)} | GEIPA2- | GEIPA | OHSteA |
| | Y3-C | | C | | |
| 1639 | Y3-Gtp-K(K(LinA)2)-Gtp-Y3 | 80/121^{(b)} | Gtp1 | Gtp | LinA |
| 1641 | Y3-Htp-K(K(LinA)2)-Htp-Y3 | 81/122^{(b)} | Htp1 | Htp | LinA |
| 1642 | Y3-Ptp-K(K(LinA)2)-Ptp-Y3 | 82/123^{(b)} | Ptp1 | Ptp | LinA |
| 1643 | Y3-Ntp-K(K(LinA)2)-Ntp-Y3 | 83/124^{(b)} | Ntp1 | Ntp | LinA |
| 1644 | Y3-Gtt-K(K(LinA)2)-Gtt-Y3 | 84/125^{(b)} | Gtt1 | Gtt | LinA |
| 1645 | Y3-GEIPA-K(K(LinA)2)-GEIPA-Y3 | 85/126^{(b)} | GEIPA1 | GEIPA | LinA |
| 1647 | Y3-H-Gtp-H-K(K(LinA)2)-H-Gtp-H-Y3 | 90/131^{(b)} | Gtp1-H | Gtp | LinA |
| 1649 | Y3-H-Htp-H-K(K(LinA)2)-H-Htp-H-Y3 | 91/132^{(b)} | Htp1-H | Htp | LinA |
| 1650 | Y3-H-Ptp-H-K(K(LinA)2)-H-Ptp-H-Y3 | 92/133^{(b)} | Ptp1-H | Ptp | LinA |
| 1651 | Y3-H-Ntp-H-K(K(LinA)2)-H-Ntp-H-Y3 | 93/134^{(b)} | Ntp1-H | Ntp | LinA |
| 1652 | Y3-H-Gtt-H-K(K(LinA)2)-H-Gtt-H-Y3 | 94/135^{(b)} | Gtt1-H | Gtt | LinA |
| 1653 | Y3-H-GEIPA-H-K(K(LinA)2)-H-GEIPA-H-Y3 | 95/136^{(b)} | GEIPA1- | GEIPA | LinA |
| | | | H | | |
| 1733 | C-Y3-chGtp2-K(K-(OHSteA)2)-chGtp2-Y3- | 76/117^{(b)} | chGtp2-C | chGtp | OHSteA |
| | C | | | | |
| 1734 | C-Y3-dGtp2-K(K-(OHSteA)2)-dGtp2-Y3-C | 77/118^{(b)} | dGtp2-C | dGtp | OHSteA |
| 1735 | C-Y3-TFEIDAtp2-K(K-(OHSteA)2)- | 78/119^{(b)} | TFEIDAtp2- | TFEIDAtp | OHSteA |
| | TFEIDAtp2-Y3-C | | C | | |
| 1736 | C-Y3-IDAtp2-K(K-(OHSteA)2)-IDAtp2-Y3-C | 79/120^{(b)} | IDAtp2-C | IDAtp | OHSteA |
| 1737 | Y3-chGtp-K(K-(LinA)2)-chGtp-Y3 | 86/127^{(b)} | chGtp1 | chGtp | LinA |
| 1738 | Y3-dGtp-K(K-(LinA)2)-dGtp-Y3 | 87/128^{(b)} | dGtp1 | dGtp | LinA |
| 1739 | Y3-TFEIDAtp-K(K-(LinA)2)-TFEIDAtp-Y3 | 88/129^{(b)} | TFEIDAtp1 | TFEIDAtp | LinA |
| 1740 | Y3-IDAtp-K(K-(LinA)2)-IDAtp-Y3 | 89/130^{(b)} | IDAtp1 | IDAtp | LinA |
| 1741 | Y3-H-chGtp-H-K(K(LinA)2)-H-chGtp-H-Y3 | 96/137^{(b)} | chGtp1-H | chGtp | LinA |
| 1742 | Y3-H-dGtp-H-K(K(LinA)2)-H-dGtp-H-Y3 | 97/138^{(b)} | dGtp1-H | dGtp | LinA |
| 1743 | Y3-H-TFEIDAtp-H-K(K(LinA)2)-H- | 98/139^{(b)} | TFEIDAtp1- | TFEIDAtp | LinA |
| | TFEIDAtp-H-Y3 | | H | | |
| 1744 | Y3-H-IDAtp-H-K(K(LinA)2)-H-IDAtp-H-Y3 | 99/140^{(b)} | DAtp1-H | IDAtp | LinA |

^{(a)α}-amino acids are indicated using the one-letter code. Stp, succinyl-tetraethylenepentamine; Gtp, glutaryl-tetraethylenepentamine; Htp, cyclohexanedicarboxyl-tetraethylenepentamine; Ptp, phthalyl-tetraethylenepentamine; Ntp, naphthalenedicarboxyl-tetraethylenepentamine; chGtp, 1,1-cyclohexanediacetyl-tetraethylenepentamine; dGtp, diglycolyl-tetraethylenepentamine; Htp, 1,2-cyclohexanedicarboxyl-tetraethylenepentamine; IDAtp, iminodiacetyl-tetraethylenepentamine; TFE-IDAtp, trifluoroethyl-iminodiacetyl-tetraethylenepentamine; Gtt, glutaryl-triethylenetetramine; GEIPA, glutaryl-N,N′-bis-(3-aminopropyl)-ethylenediamine; OleA, oleic acid; OHSteA, mono-hydroxylated stearic acid; LinA, linoleic acid; LenA, linolenic acid; SteA, stearic acid; CholA, cholanic acid.
^{(b)}All sequences were synthesized and analyzed with an N-terminal azido lysine (K(N₃)).

### Analytical Data

The various sequence defined oligomers (see Table 1) were analyzed using MALDI-TOF mass spectrometry. In brief, matrix solution containing 10 mg/mL Super-DHB (90/10 m/m mixture of 2,5-dihydroxybenzoic c acid and 2-hydroxy-5-methoxybenzoic acid) in 69.93/30/0.07 (v/V/V) H₂O/acetonitrile/trifluoroacetic acid was spotted on an MTP Anchor Chip (Bruker Daltonics, Germany). After crystallization of 1 μL matrix solution, 1 μL of sample solution (10 mg/ml in water) was added to the matrix spot. Samples were analyzed using an Autoflex II mass spectrometer (Bruker Daltonics, Germany). All spectra were recorded in positive ion mode.

### 1H Nuclear Magnetic Resonance (¹H NMR) Spectroscopy

¹H NMR spectra were recorded on an Advance III HD Bruker BioSpin (400 MHZ) in chloroform-d (CDCl₃), deuterium oxide (D₂O) or methanol-d4 (CD₃OD) without TMS. Chemical shifts were calibrated to the residual solvent signal and were reported in parts per million (ppm). The spectra were processed in MestReNova software (MestReLab Research, SL). Integration was performed manually.

### Electrospray Ionization Mass Spectroscopy (ESI-MS)

ESI mass spectra of artificial building blocks were recorded with a Thermo Scientific LTQ FT Ultra fourier transform ion cyclotron and an lonMax source in chloroform or methanol.

### Dynamic Light Scattering (DLS) and Zeta Potential Analysis

The hydrodynamic particle size and zeta potential of nanoparticles were measured in folded capillary cells (DTS 1070) using a Zetasizer Nano ZS (Malvern Instruments, UK). The scattering angle of 173° was fixed, the refractive index (RI) of the solvent was 1.330, the viscosity was 0.8872 mPa·s, and the temperature was set to 25° C. All samples were measured 3 times with 12-15 sub-runs.

### Determination of Half Maximal Effective Concentration (EC50)

The eGFP knockout studies were carried out with a series of concentrations of RNP (100, 75, 50, 25, 10, 5, 2.5, 1, 0.5, and 0.1 nM). Each concentration was used in triplicates. Based on the obtained dose-response correlation, half maximal effective concentration (EC50) values were calculated by GraphPad prism 5.

LogD74 determination of artificial amino acids

Octanol-water partition coefficient D at pH 7.4 (logD7.4) was determined as previously reported (H. J. Kim, S. Ogura, T. Otabe, R. Kamegawa, M. Sato, K. Kataoka, K. Miyata, ACS Cent. Sci. 2019, 5, 1866-1875 and J. Yum, B. S. Kim, S. Ogura, R. Kamegawa, M. Naito, Y. Yamasaki, H. J. Kim, K. Miyata, J. Controlled Release 2022, 342, 148-156) with slight modification. 10 μL of a solution containing 10 mg/ml lipo-oligomer in water was diluted with 90 μL HEPES buffer (20 mM, pH 7.4) in 1.5 mL reaction tubes. 100 μL n-octanol were then added. The tubes were shaken at 1400 rpm for 24 h. Afterwards, the tubes were centrifuged followed by storage at 4° C. for 1 h to separate n-octanol and water phases. The concentration of lipo-oligomer in each phase was determined by RP-HPLC analysis. For this, a VWR Hitachi Chromaster HPLC system equipped with a 5160 pump module, a 5260 auto sampler, a 5310 column oven, and a 5430 diode array detector, a YMC C18 column (HS-302, HS12805-1546WT, 5 μm, 4.6×150 mm, 12 nm, YMC Europe GmbH, Dinslaken, Germany) and a water/acetonitrile gradient (95:5-0:100) containing 0.1% TFA were used. The extinction at 280 nm was monitored and the peak areas (A) of the oligoamino amides were determined. LogD7.4 was calculated as log (A_{octanol}/A_{water}). All experiments were performed in triplicates. EC50-logD7.4 correlation was analyzed with a second order polynomial equation by GraphPad prism 5.

sgRNA Sequences

Chemically modified sgRNAs with the following sequences were obtained from Integrated DNA Technologies IDT (Leuven, Belgium).

| sgRNA/ssD | |
|---|---|
| NA | Sequences (5′→3′) |
| sgGFP1^{[a]} | mG*mA*mC*rCrArGrGrArUrGrGrGrCrArCrCrArCrCrCrGrUrUrUrUrArGrArGrCrUrArGrArArAr |
| | UrArGrCrArArGrUrUrArArArArUrArArGrGrCrUrArGrUrCrCrGrUrUrArUrCrArArCrUrUrGrArArA |
| | rArArGrUrGrGrCrArCrCrGrArGrUrCrGrGrUrGrCmU*mU*mU*rU (SEQ ID NO: 108) |
| sgGFP2^{[b]} | mG*mC*mU*rGrArArGrCrArCrUrGrCrArCrGrCrCrGrUrGrUrUrUrUrArGrArGrCrUrArGrArArAr |
| | UrArGrCrArArGrUrUrArArArArUrArArGrGrCrUrArGrUrCrCrGrUrUrArUrCrArArCrUrUrGrArArA |
| | rArArGrUrGrGrCrArCrCrGrArGrUrCrGrGrUrGrCmU*mU*mU*rU (SEQ ID NO: 109) |
| SSDNA | G*C*CACCTACGGCAAGCTGACCCTGAAGTTCATCTGCACCACCGGCAAGCTGCCCGTG |
| | CCCTGGCCCACCCTCGTGACCACCCTGAGCCACGGCGTGCAGTGCTTCAGCCGCTACC |
| | CCGACCACAT*G*A (SEQ ID NO: 110) |

^{[a]}sgGFP1 was used in eGFP knockout experiments.
^{[b]}sgGFP2 was used together with the ssDNA template in homology-directed repair (HDR) mediated eGFP to BFP conversion experiments;
‘m’ indicates 2′-O-methylated nucleosides;
***indicates phosphorothioate linkages and ‘r’ indicates ribonucleotides.

Preparation of Cas9 RNP and Cas9 RNP/ssDNA nanocarriers

Cas9 protein expression and purification was performed as described in Kuhn et al., (Bioconjugate Chemistry, 2020, 31.3:729-742). Cas9 protein and sgRNA were mixed at 1:1 molar ratio and incubated at RT for 15 min to form the Cas9/sgRNA ribonucleoprotein (RNP) complexes. Afterwards, Cas9 RNP was added into the lipo-oligomer solution in HBG buffer (20 mM HEPES, 5% glucose, pH 7.4) at an N/P (nitrogen to phosphate of sgRNA) ratio of 24. The solution was mixed thoroughly by pipetting up and down and incubated at RT for another 15 min to generate the final Cas9 RNP nanocarriers. In case of preparing Cas9 RNP/ssDNA nanocarriers for HDR experiments, Cas9 RNP was mixed with ssDNA HDR template at 1:1 molar ratio and then added into the lipopeptide solution in HBG buffer (pH 7.4) at an N/P (nitrogen to phosphate of sgRNA plus ssDNA) ratio of 12. The solution was mixed and incubated for 40 min to generate the Cas9 RNP/ssDNA nanocarriers.

### Characterization of Cas9 RNP Nanocarriers

The hydrodynamic size, PDI and zeta potential of Cas9 RNP nanocarriers were measured by DLS using a Zetasizer Nano ZS (Malvern Instruments, UK). Cas9 RNP nanocarriers were prepared as described above. The hydrodynamic size of Cas9 RNP nanocarriers containing 1.25 μg Cas9 protein and 0.25 μg sgRNA (Cas9/sgRNA, 1:1 molar ratio) in 100 UL HBG was measured. Afterwards, 700 μL HEPES buffer (20 mM, pH 7.4) were added to each sample and the solution was mixed for the zeta potential measurement. For the dilution stability study, Cas9 RNP nanocarriers were prepared at an RNP concentration of 375 nM (6.25 μg Cas9 protein and 1.25 μg sgRNA) and then diluted with HBG buffer to a series of RNP concentrations (75, 50, 25, 10, 5, 2.5, 1, 0.5, and 0.1 nM). The size and zeta potential of diluted samples were measured as described above.

### Ribogreen Assay

sgRNA amount in naked Cas9/sgRNA ribonucleoproteins (Cas9 RNPs) or Cas9 RNP nanoparticles were determined using a Quant-IT™ RiboGreen® RNA assay kit (Invitrogen, CA, USA). 50 μL of naked Cas9 RNPs or Cas9 RNP nanoparticles containing 250 ng Cas9 protein and 50 ng sgRNA were mixed with 50 μL of TE buffer and were added to the 96-well plate. The plate was incubated for 10 min. Afterwards, 100 μL of Ribogreen solution (diluted 1:100 in TE buffer) was added to each well. The plate was incubated for another 5 min. Ribogreen fluorescence intensity was detected using a microplate reader (Spectrafluor Plus, Tecan, Männedorf, Switzerland) with excitation and emission wavelength of 485 and 528 nm, respectively.

### Heparin Competition Assay

Heparin competition assay was performed to evaluate the nanocarrier stability against anionic stress. 50 μL TE buffer containing different amounts of Heparin were added to the 96-well plate. 50 μL of Cas9 RNP nanocarriers containing 250 ng Cas9 protein and 50 ng sgRNA were prepared and added to each well. The final amounts of Heparin were 0, 0.25, 0.5, 1, 2.5, 5 IU per μg of sgRNA. The plate was incubated at 37° C. for 30 min. Afterwards, 100 μL Ribogreen solution was added and a Ribogreen assay was performed. HBG buffer and naked Cas9 RNP at the same conditions were prepared as the controls. After subtraction of the blank wells (HBG buffer), the fraction of dye exclusion was calculated as [1-fraction of Ribogreen intercalation (normalized to Ribogreen intercalation of naked Cas9/sgRNA RNP)]. All experiments were performed in triplicate.

### Nanocarrier Stability in Salt Conditions

Nanocarrier stability against salt was studied using NaCl solutions. 50 μL TE buffer containing different concentrations of NaCl were added to the 96-well plate. 50 μL of Cas9 RNP nanocarriers containing 250 ng Cas9 protein and 50 ng sgRNA were prepared and added to each well. The final concentrations of NaCl were 0, 0.05, 0.15, 0.25, 0.5, 5 M. The plate was incubated at 37° C. for 30 min. Afterwards, 100 μL Ribogreen solution was added and a Ribogreen assay was performed. HBG buffer and naked Cas9 RNP at the same conditions were prepared as the controls. After subtraction of the blank wells (HBG buffer), the fraction of dye exclusion was calculated as [1-fraction of Ribogreen intercalation (normalized to Ribogreen intercalation of naked Cas9/sgRNA RNP)]. All experiments were performed in triplicate.

### Establishment of HeLa GFPd2 Cell Line

HeLa cells were transfected using a Super piggyBac Transposase expression vector (SBI, CA, USA) and a PB-CAG-GFPd2 plasmid (Rui et al., ACS Appl Mater Interfaces, 11 (11), 2019, 10472-10480). PB-CAG-GFPd2 was a gift from Jordan Green (Addgene plasmid #115665; http://n2t.net/addgene: 115665; RRID: Addgene_115665). Afterwards, the cells were incubated and passaged twice a week for 3 weeks, allowing the GFP fluorescence from transient transfections to fade. Subsequently, GFP-positive cells were sorted and collected by fluorescence-assisted cell sorting (FACS). The colonies from single GFP-positive cells were generated using limited dilution method. A stably GFP-expressing cell population was selected from the regrown colonies by flow cytometry.

Establishment of HeLa eGFP/Tub Cell Line

For the generation of stably expressing HeLa eGFP/tub cells, the plasmid vector pEGFP-Tubulin (Clontech Laboratories) was linearized by restriction digestion and transfected into the cells. The vector contains a neomycin resistance cassette, which enables selection of stably transfected cells by cultivation with G418. Single colonies from the surviving cell populations were generated using limited dilution method. Cell populations generated from the regrown clones were analyzed by flow cytometry and a monoclonal cell lines with stable eGFP expression was selected.

### Cell Culture

HeLa WT, HeLa eGFP/tub, and HeLa GFPd2 (destabilized eGFP) cells were grown in DMEM medium supplemented with 10% FBS, 100 U/mL penicillin, and 100 μg/mL streptomycin. The cells were cultured in ventilated flasks in the cell incubator at 37° C. and 5% CO₂ in a humidified atmosphere. The cells were passaged at a confluency of approximately 80%.

### Flow Cytometry

Cells were harvested and resuspended in 600 μL of FACS buffer (PBS buffer containing 10% FBS). The samples were analyzed by flow cytometry on a CytoFLEX S flow cytometer (Beckman Coulter, CA, USA). For eGFP knockout and cellular uptake experiments, 0.6 μL of 1 μg/μL DAPI was added to each sample for differentiating live and dead cells before the measurement. The DAPI signal and BFP fluorescence were detected with 405 nm excitation and 450 nm emission. The eGFP and ATTO488 fluorescence was assayed with 488 nm excitation and 530 nm emission. The ATTO647N fluorescence was assayed with 640 nm excitation and 670 nm emission. Ten thousand of isolated live cells were counted and evaluated. The data were analyzed using FlowJo 7.6.5 by FlowJo, LLC (Becton, Dickinson and Company, USA).

### Cellular Uptake of Cas9 RNP Nanocarriers

Cellular uptake of Cas9 RNP nanocarriers was evaluated by flow cytometry and confocal laser scanning microscopy (CLSM). For flow cytometry experiments, HeLa WT cells were seeded into 24-well plates at a density of 25000 cells/well one day prior to the treatments. On the next day, the medium was replaced with 400 μL of fresh medium. Cas9 RNP nanocarriers were prepared as described above using 20% of ATTO647N-labeled Cas9 and 20% of ATTO488-labeled sgRNA. 100 μL of RNP nanocarriers were added to each well resulting in a final concentration of 75 nM Cas9 RNP followed by incubation of the cells for 4 h. Afterwards, the medium was removed and 500 μL PBS containing 2000 IU heparin was added to disassociate the nanocarriers attached to the cell membrane. The cells were incubated on ice for 30 min. Subsequently, the cells were collected and prepared for flow cytometry analysis on a CytoFLEX S flow cytometer (Beckman Coulter, CA, USA). All experiments were performed in triplicate.

### Endocytosis Pathway Study

Endocytosis pathway of Cas9 RNP nanocarriers with varied hydrophobicity was investigated by using different endocytosis inhibitors. One day prior to the treatments, HeLa WT cells were seeded into 24-well plates at a density of 25000 cells/well. On the next day, the medium was replaced with 500 μL of serum-free medium containing different endocytosis inhibitors (15.4 mM sodium azide, 10 UM chlorpromazine, 450 mM sucrose, 54 μM nystatin, or 1 mM amiloride). The cells were incubated at 37° C. for 2 h. Afterwards, the medium was replaced with 400 μL of fresh medium. 100 μL of dye-labeled RNP nanocarriers were added to each well resulting in a final concentration of 75 nM Cas9 RNP followed by incubation of the cells for 4 h. For 4° C. group, the cells were placed on ice for 2 h prior to the treatment of 75 nM Cas9 RNP in ice-cold medium. Afterwards, the medium was removed and 500 μL PBS containing 2000 IU heparin was added followed by incubation of the cells on ice for 30 min. The cells were then collected and prepared for flow cytometry analysis on a CytoFLEX S flow cytometer (Beckman Coulter, CA, USA). All experiments were performed in triplicate.

Description eGFP Knockout Experiment

One day prior to eGFP knockout experiment, HeLa eGFP/tub cells were seeded into 96-well plates at a density of 5000 cells/well. On the next day, the medium in each well was replaced with 80 μL of fresh medium. Cas9 protein was mixed with sgGFP1 at a molar ratio of 1:1 and the RNP complexes were incubated for 15 min at room temperature. Cas9 RNP nanoparticles were prepared by mixing pre-assembled Cas9 RNP complexes with oligomers at N/P ratio of 24 at RNP dose of 500 nM and then diluted to a series of concentrations (375, 250, 125, 50, 25, 12.5, 5, 2.5, and 0.5 nM) with HBG buffer. The N/P ratio defines the lipo-oligomer nitrogen (N) to sgRNA phosphate (P) ratio. The RNP nanoparticles were incubated for 15 min at room temperature. 20 μL of RNP nanoparticles with different concentrations was added to each well resulting in final concentrations of 100, 75, 50, 25, 10, 5, 2.5, 1, 0.5, and 0.1 nM RNP complexes. The cells were incubated for 48 h. Afterwards, the cells were trypsinized and transferred to the 24-well plates. After another 3 days incubation, the cells were harvested and resuspended in 600 μL of FACS buffer. The samples were analyzed by flow cytometry on a CytoFLEX S flow cytometer (Beckman Coulter, CA, USA). Before the measurement, 1 ng/μL DAPI was added to differentiate between live and dead cells. The DAPI signal was detected with 405 nm excitation and 450 nm emission. The eGFP fluorescence was assayed with 488 nm excitation and 530 nm emission. Ten thousand of isolated live cells were counted and evaluated. The data were analyzed using FlowJo 7.6.5 by FlowJo, LLC (Becton, Dickinson and Company, USA). All experiments were performed in triplicate.

### Description Homology-Directed Repair (HDR) Experiment

A single base substitution (196 T>C) in the chromophore of wild-type (wt) green fluorescent protein (GFP) shifts its fluorescent absorption and emission towards the blue spectrum, creating the blue fluorescent protein (BFP). This can be exploited to detect CRISPR/Cas9 mediated HDR.

One day prior to GFP to BFP HDR experiment, HeLa GFPd2 cells were seeded into 96-well plates at a density of 5000 cells/well. On the next day, the medium in each well was replaced with 80 μL of fresh medium. Pre-assembled Cas9/sgGFP RNP was mixed with single stranded DNA template (ssDNA) at various molar ratios (1:0.5, 1:1, 1:2, 1:3, 1:4, 1:5, 1:8, 1:10, 1:15, and 1:20). Cas9 RNP/ssDNA nanoparticles were prepared by mixing Cas9 RNP/ssDNA complexes with lipo-oligomers at N/P ratio of 12 at RNP dose of 500 nM and then diluted to a series of concentrations (375, 250, 125, 50, 25, 12.5, 5, 2.5, and 0.5 nM) with HBG buffer (20 mM HEPES, 5% glucose, pH 7.4). The RNP/ssDNA nanoparticles were incubated for 45 min at room temperature. 20 μL of RNP/ssDNA nanoparticles with different concentrations was added to each well resulting in final concentrations of 100, 75, 50, 25, 10, 5, 2.5, 1, 0.5, and 0.1 nM RNP complexes. The cells were incubated for 48 h. Afterwards, the cells were trypsinized and transferred to the 24-well plates. After another 3 days incubation, the cells were harvested and resuspended in 600 μL of FACS buffer. The samples were analyzed by flow cytometry on a CytoFLEX S flow cytometer (Beckman Coulter, CA, USA). The BFP fluorescence was detected with 405 nm excitation and 450 nm emission. The GFP fluorescence was assayed with 488 nm excitation and 530 nm emission. Ten thousand of cells were counted and evaluated. The data were analyzed using FlowJo 7.6.5 by FlowJo, LLC (Becton, Dickinson and Company, USA). All experiments were performed in triplicate.

### Cell Viability Assay (MTT)

Treatments were performed as described in eGFP knockout and HDR studies. The cells were incubated for 48 h and an MTT assay was then carried out. 10 μL of MTT (5 mg/mL) was added to each well. Cells were incubated for 2 h. Afterwards, medium was removed and the plate was frozen at −80° C. for 4 h. 100 μL of DMSO was added to each well. The plate was incubated for another 30 min at 37° C. under constant shaking. The absorbance was measured at 590 nm with background correction at 630 nm using a fluorescence microplate reader (Tecan, Männedorf, Switzerland). The relative cell viability (%) was calculated relative to control wells treated with HBG buffer as ([A] test/[A] control)×100%. All experiments were performed in triplicate.

### Statistical Analysis

Half maximal effective concentration (EC50) values were calculated by GraphPad prism 5. EC50-logD7.4 correlation was analyzed with a second order polynomial equation by GraphPad prism 5. All other data were analyzed with GraphPad prism 5 and presented as arithmetic mean±standard deviation (SD) of at least triplicates. The statistical significance of the experiments was estimated using the two-tailed student's t-test, *** p≤0.001, ** p≤0.01, * p≤0.05.

### Example 1: Oligomers with Optimized Sequences

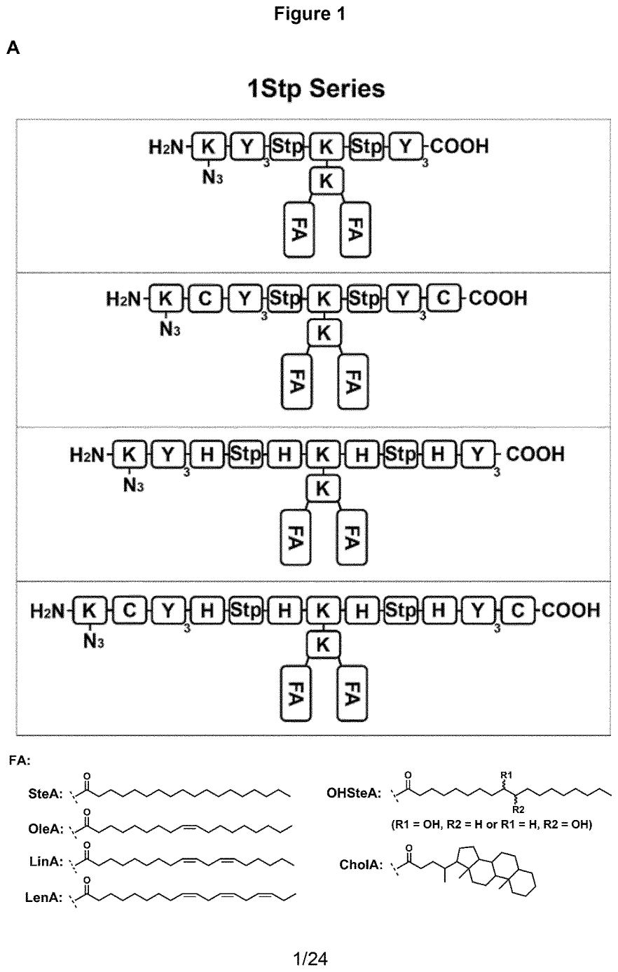

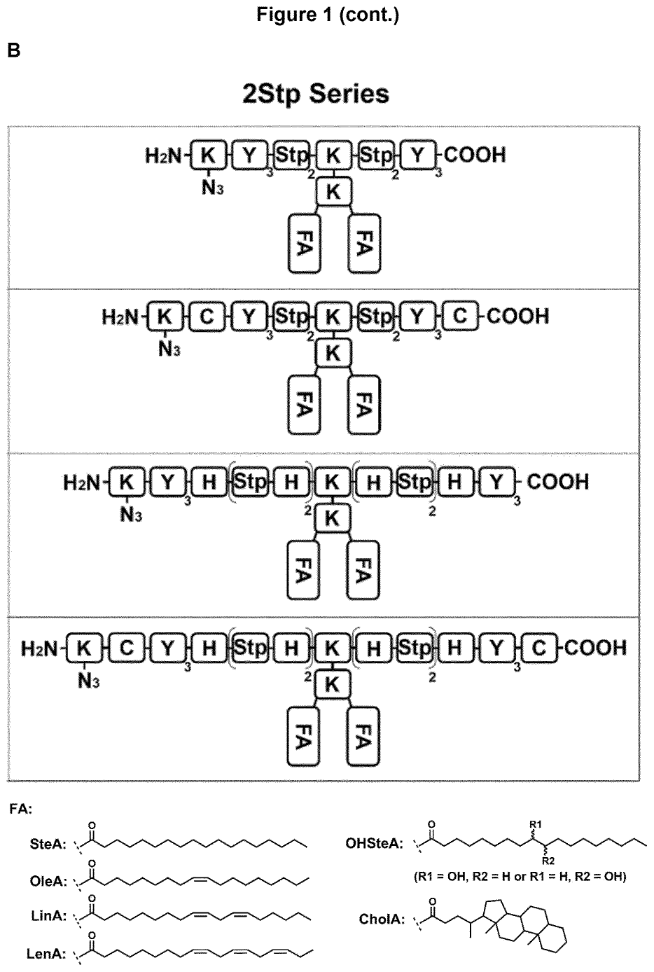

Sequence-defined oligomers with different sequences based on α-amino acids and the artificial amino acid succinyl-tetraethylenepentamine and fatty acids were generated to identify optimized sequence-defined T-shape lipo-oligomers for Cas protein/gRNA RNP complex delivery, such as oligomer IDs #1208 to 1591 as shown in Table 1, including the already known structure 1445 (Stp2-C-OHSteA) as described in Kuhn et al., (Kuhn, J. et al., Delivery of Cas9/sgRNA Ribonucleoprotein Complexes via Hydroxystearyl Oligoamino Amides, Bioconjugate Chemistry, 2020, 31, 729-742). These sequence-defined oligomers are summarized and illustrated as 1Stp series and 2Stp series in FIG. 1 showing the lead compound Stp as representative artificial amino acid. The core structures of the 1Stp and 2Stp series differ in the presence of one or two Stp on either side of the central lysine (either chain (N-terminal and C-terminal) of the head group).

The artificial amino acid, succinyl-tetraethylenepentamine (Stp), serves as an ionizable unit to complex negatively charged cargo, such as Cas9 RNP, and facilitate cellular delivery (D. Schaffert et al., Organic Letters 2011, 13, 1586-1589; D. Schaffert et al., Angewandte Chemie International Edition 2011, 50, 8986-8989; Krhač Levačić et al., Journal of Controlled Release 2021, 339, 27-40; T. Benli-Hoppe et al., Macromolecular Rapid Communications 2021, n/a, 2100602; J. Luo et al., Advanced Functional Materials 2019, 29, 1900697). The hydrophobic tyrosine (Y) tripeptide motif enhances nanoparticle stability via hydrophobic interactions and improves transfection efficiency (C. Troiber et al., Biomaterials 2013, 34, 1624-1633; S. Berger et al., Biomacromolecules 2021, 22, 1282-1296). Here, the lead structure Stp2-C-OHSteA (oligomer ID #1445) has been varied systematically to elucidate structure-activity relationships and evolve the next generation of RNP carriers. Variations of the structural parameters of 1) presence of cysteine (C), 2) presence of histidine (H), 3) number of Stp units, and 4) type of fatty acid were introduced into the peptide sequence (FIG. 1). All tested compounds comprised an N-terminal azido-lysine which can be used for coupling of a targeting ligand, but has no function with regard to the cargo delivery and/or nanoparticle stability. The Cas9 RNP nanoparticles were prepared by straightforward complexation of the lipopeptides with Cas9 RNP at an N/P ratio of 24. The hydrodynamic size, polydispersity index (PDI), and zeta potential of each nanoparticle were then determined by dynamic light scattering (DLS, Table 2). Most lipopeptides generated RNP nanoparticles with a size between 150-250 nm and PDI of 0.15-0.40, except Stp2 and Stp2-H structures, which led to larger complexes (250-500 nm). Besides, all nanoparticles exhibited a positive surface charge with a zeta potential between 10-19 mV

TABLE 2
ID number, sequence, MALDI-MS of all lipopeptides and hydrodynamic size, PDI, zeta potential of Cas9 RNP nanoparticles
formed with the lipopeptides (Cas9 RNP nanoparticles were prepared at a N/P ratio of 24 and an RNP concentration of
75 nM. The hydrodynamic size (z-average, nm), PDI and zeta potential of the nanoparticles were measured by DLS using a Zetasizer Nano ZS
(Malvern Instruments, UK)).

| | | | | Zeta | MS | MS |
|---|---|---|---|---|---|---|
| ID | Sequence (N→C)* | Size^{[a]} | PDI^{[a]} | potential^{[a]} | [calc.] | [found] |
| 1468 | Y3-Stp-K(K(SteA)2)-Stp-Y3 | 201.5 ± 2.8 | 0.26 ± 0.03 | 15.2 ± 1.1 | 2481.59 | 2480.21 |
| 1391 | Y3-Stp-K(K(OleA)2)-Stp-Y3 | 152.3 ± 3.8 | 0.29 ± 0.03 | 12.6 ± 1.9 | 2477.56 | 2473.18 |
| 1392 | Y3-Stp-K(K(LinA)2)-Stp-Y3 | 159.3 ± 1.5 | 0.20 ± 0.04 | 12.8 ± 0.4 | 2473.53 | 2477.58 |
| 1393 | Y3-Stp-K(K(LenA)2)-Stp-Y3 | 173.9 ± 4.0 | 0.27 ± 0.03 | 11.7 ± 0.9 | 2469.50 | 2463.12 |
| 1394 | Y3-Stp-K(K(CholA)2)-Stp-Y3 | 194.4 ± 1.0 | 0.38 ± 0.03 | 14.4 ± 1.3 | 2633.65 | 2630.41 |
| 1469 | Y3-Stp-K(K(OHSteA)2)-Stp-Y3 | 161.2 ± 4.1 | 0.26 ± 0.03 | 13.4 ± 0.2 | 2513.58 | 2510.21 |
| 1470 | Y3-H-Stp-H-K(K(SteA)2)-H-Stp-H-Y3 | 204.9 ± 5.7 | 0.16 ± 0.04 | 14.9 ± 1.9 | 3029.83 | 3024.77 |
| 1395 | Y3-H-Stp-H-K(K(OleA)2)-H-Stp-H-Y3 | 200.2 ± 3.5 | 0.19 ± 0.02 | 11.0 ± 1.4 | 3025.79 | 3020.04 |
| 1396 | Y3-H-Stp-H-K(K(LinA)2)-H-Stp-H-Y3 | 194.9 ± 3.8 | 0.22 ± 0.03 | 10.3 ± 1.3 | 3021.76 | 3021.27 |
| 1397 | Y3-H-Stp-H-K(K(LenA)2)-H-Stp-H-Y3 | 185.7 ± 0.9 | 0.35 ± 0.03 | 12.2 ± 1.5 | 3017.73 | 3013.67 |
| 1398 | Y3-H-Stp-H-K(K(CholA)2)-H-Stp-H-Y3 | 167.1 ± 5.7 | 0.15 ± 0.03 | 16.1 ± 1.2 | 3181.89 | 3177.96 |
| 1471 | Y3-H-Stp-H-K(K(OHSteA)2)-H-Stp-H-Y3 | 193.8 ± 4.4 | 0.22 ± 0.02 | 16.0 ± 0.6 | 3061.81 | 3055.77 |
| 1581 | C-Y3-Stp-K(K(SteA)2)-Stp-Y3-C | 190.3 ± 5.2 | 0.31 ± 0.01 | 15.2 ± 1.6 | 2687.61 | 2683.51 |
| 1577 | C-Y3-Stp-K(K(OleA)2)-Stp-Y3-C | 156.2 ± 3.9 | 0.40 ± 0.05 | 13.2 ± 1.7 | 2683.58 | 2678.47 |
| 1578 | C-Y3-Stp-K(K(LinA)2)-Stp-Y3-C | 147.0 ± 3.4 | 0.16 ± 0.02 | 14.0 ± 0.2 | 2679.55 | 2673.43 |
| 1579 | C-Y3-Stp-K(K(LenA)2)-Stp-Y3-C | 151.5 ± 6.8 | 0.29 ± 0.03 | 11.5 ± 0.2 | 2675.51 | 2671.4 |
| 1580 | C-Y3-Stp-K(K(CholA)2)-Stp-Y3-C | 190.1 ± 6.0 | 0.19 ± 0.03 | 14.1 ± 0.8 | 2839.67 | 2833.69 |
| 1582 | C-Y3-Stp-K(K(OHSteA)2)-Stp-Y3-C | 180.2 ± 4.3 | 0.24 ± 0.02 | 12.2 ± 1.2 | 2719.60 | 2713.53 |
| 1587 | C-Y3-H-Stp-H-K(K(SteA)2)-H-Stp-H-Y3-C | 245.1 ± 10.3 | 0.27 ± 0.02 | 14.1 ± 0.1 | 3235.84 | 3230.05 |
| 1583 | C-Y3-H-Stp-H-K(K(OleA)2)-H-Stp-H-Y3-C | 195.3 ± 5.8 | 0.15 ± 0.01 | 12.3 ± 1.9 | 3231.81 | 3228.02 |
| 1584 | C-Y3-H-Stp-H-K(K(LinA)2)-H-Stp-H-Y3-C | 235.8 ± 9.5 | 0.23 ± 0.04 | 12.8 ± 1.7 | 3227.78 | 3223.99 |
| 1585 | C-Y3-H-Stp-H-K(K(LenA)2)-H-Stp-H-Y3-C | 202.4 ± 8.7 | 0.39 ± 0.04 | 13.4 ± 0.7 | 3223.75 | 3218.96 |
| 1586 | C-Y3-H-Stp-H-K(K(CholA)2)-H-Stp-H-Y3-C | 199.0 ± 8.6 | 0.36 ± 0.03 | 16.1 ± 0.6 | 3387.91 | 3383.24 |
| 1588 | C-Y3-H-Stp-H-K(K(OHSteA)2)-H-Stp-H-Y3-C | 190.1 ± 6.0 | 0.20 ± 0.03 | 12.9 ± 0.7 | 3267.83 | 3261.05 |
| 1361 | Y3-Stp2-K(K(SteA)2)-Stp2-Y3 | 377.5 ± 56.6 | 0.37 ± 0.01 | 17.5 ± 1.4 | 3023.99 | 3017.93 |
| 1208 | Y3-Stp2-K(K(OleA)2)-Stp2-Y3 | 379.2 ± 70.5 | 0.29 ± 0.03 | 14.1 ± 0.4 | 3019.96 | 3013.69 |
| 1399 | Y3-Stp2-K(K(LinA)2)-Stp2-Y3 | 345.5 ± 33.8 | 0.29 ± 0.01 | 13.1 ± 0.9 | 3015.93 | 3011.87 |
| 1400 | Y3-Stp2-K(K(LenA)2)-Stp2-Y3 | 385.0 ± 47.5 | 0.35 ± 0.03 | 14.3 ± 1.6 | 3011.90 | 3005.84 |
| 1364 | Y3-Stp2-K(K(CholA)2)-Stp2-Y3 | 501.6 ± 52.6 | 0.41 ± 0.01 | 13.0 ± 1.9 | 3176.05 | 3170.12 |
| 1573 | Y3-Stp2-K(K(OHSteA)2)-Stp2-Y3 | 440.3 ± 33.2 | 0.24 ± 0.01 | 14.3 ± 0.3 | 3055.98 | 3051.93 |
| 1321 | Y3-(H-Stp)2-H-K(K(SteA)2)-H-(Stp-H)2-Y3 | 340.2 ± 31.2 | 0.16 ± 0.04 | 14.3 ± 1.5 | 3846.34 | 3840.77 |
| 1209 | Y3-(H-Stp)2-H-K(K(OleA)2)-H-(Stp-H)2-Y3 | 338.3 ± 28.0 | 0.28 ± 0.05 | 14.9 ± 0.5 | 3842.31 | 3836.73 |
| 1401 | Y3-(H-Stp)2-H-K(K(LinA)2)-H-(Stp-H)2-Y3 | 478.7 ± 8.1 | 0.16 ± 0.02 | 11.1 ± 1.5 | 3838.28 | 3832.71 |
| 1402 | Y3-(H-Stp)2-H-K(K(LenA)2)-H-(Stp-H)2-Y3 | 288.5 ± 17.5 | 0.45 ± 0.04 | 11.9 ± 0.5 | 3834.25 | 3830.67 |
| 1403 | Y3-(H-Stp)2-H-K(K(CholA)2)-H-(Stp-H)2-Y3 | 366.8 ± 37.7 | 0.40 ± 0.04 | 13.9 ± 1.0 | 3998.41 | 3995.96 |
| 1574 | Y3-(H-Stp)2-H-K(K(OHSteA)2)-H-(Stp-H)2-Y3 | 256.4 ± 11.5 | 0.37 ± 0.03 | 12.4 ± 0.3 | 3878.33 | 3872.76 |
| 1337 | C-Y3-Stp2-K(K(SteA)2)-Stp2-Y3-C | 201.4 ± 10.6 | 0.29 ± 0.03 | 16.7 ± 1.9 | 3230.01 | 3226.22 |
| 1338 | C-Y3-Stp2-K(K(OleA)2)-Stp2-Y3-C | 166.6 ± 5.8 | 0.25 ± 0.03 | 16.6 ± 0.4 | 3225.98 | 3221.18 |
| 1199 | C-Y3-Stp2-K(K(LinA)2)-Stp2-Y3-C | 159.3 ± 5.9 | 0.23 ± 0.02 | 15.9 ± 1.3 | 3221.95 | 3217.15 |
| 1200 | C-Y3-Stp2-K(K(LenA)2)-Stp2-Y3-C | 160.6 ± 4.3 | 0.17 ± 0.03 | 16.8 ± 1.2 | 3217.92 | 3211.12 |
| 1340 | C-Y3-Stp2-K(K(CholA)2)-Stp2-Y3-C | 203.4 ± 6.9 | 0.28 ± 0.03 | 15.4 ± 0.8 | 3382.07 | 3376.41 |
| 1445 | C-Y3-Stp2-K(K(OHSteA)2)-Stp2-Y3-C | 177.7 ± 5.9 | 0.25 ± 0.04 | 16.5 ± 1.7 | 3262.00 | 3256.56 |
| 1278 | C-Y3-(H-Stp)2-H-K(K(SteA)2)-H-(Stp-H)2-Y3-C | 205.1 ± 12.9 | 0.20 ± 0.04 | 15.1 ± 1.1 | 4052.36 | 4048.05 |
| 1214 | C-Y3-(H-Stp)2-H-K(K(OleA)2)-H-(Stp-H)2-Y3-C | 185.5 ± 5.7 | 0.26 ± 0.05 | 18.5 ± 1.5 | 4048.33 | 4042.02 |
| 1389 | C-Y3-(H-Stp)2-H-K(K(LinA)2)-H-(Stp-H)2-Y3-C | 178.5 ± 10.2 | 0.30 ± 0.02 | 16.8 ± 1.2 | 4044.30 | 4038.99 |
| 1390 | C-Y3-(H-Stp)2-H-K(K(LenA)2)-H-(Stp-H)2-Y3-C | 188.8 ± 9.9 | 0.28 ± 0.02 | 15.8 ± 0.6 | 4042.96 | 4036.96 |
| 1575 | C-Y3-(H-Stp)2-H-K(K(CholA)2)-H-(Stp-H)2-Y3-C | 200.7 ± 10.3 | 0.33 ± 0.03 | 14.7 ± 1.4 | 4204.43 | 4200.25 |
| 1576 | C-Y3-(H-Stp)2-H-K(K(OHSteA)2)-H-(Stp-H)2-Y3-C | 191.6 ± 6.3 | 0.33 ± 0.07 | 14.6 ± 0.6 | 4084.35 | 4078.06 |
| 1639 | Y3-Gtp-K(K(LinA)2)-Gtp-Y3 | 156.3 ± 2.9 | 0.15 ± 0.04 | 13.1 ± 0.8 | 2501.56 | 2497.32 |
| 1641 | Y3-Htp-K(K(LinA)2)-Htp-Y3 | 165.3 ± 2.7 | 0.22 ± 0.05 | 12.1 ± 0.9 | 2581.62 | 2577.31 |
| 1642 | Y3-Ptp-K(K(LinA)2)-Ptp-Y3 | 168.1 ± 3.2 | 0.33 ± 0.01 | 10.9 ± 1.9 | 2569.53 | 2573.76 |
| 1643 | Y3-Ntp-K(K(LinA)2)-Ntp-Y3 | 175.6 ± 1.1 | 0.26 ± 0.02 | 11.3 ± 1.3 | 2669.56 | 2663.09 |
| 1644 | Y3-Gtt-K(K(LinA)2)-Gtt-Y3 | 165.3 ± 10.0 | 0.22 ± 0.03 | 11.5 ± 0.9 | 2415.47 | 2410.15 |
| 1645 | Y3-GEIPA-K(K(LinA)2)-GEIPA-Y3 | 169.3 ± 9.1 | 0.28 ± 0.04 | 11.6 ± 0.8 | 2471.54 | 2467.39 |
| 1737 | Y3-chGtp-K(K(LinA)2)-chGtp-Y3 | 176.3 ± 3.5 | 0.31 ± 0.05 | 12.5 ± 1.9 | 2637.68 | 2634.49 |
| 1738 | Y3-dGtp-K(K(LinA)2)-dGtp-Y3 | 173.5 ± 1.3 | 0.20 ± 0.03 | 13.9 ± 2.9 | 2505.52 | 2501.42 |
| 1739 | Y3-(TFE-IDAtp)-K(K(LinA)2)-(TFE-IDAtp)-Y3 | 166.3 ± 6.1 | 0.29 ± 0.03 | 12.5 ± 1.1 | 2667.55 | 2660.05 |
| 1740 | Y3-IDAtp-K(K(LinA)2)-IDAtp-Y3 | 138.9 ± 9.0 | 0.31 ± 0.03 | 13.6 ± 3.1 | 2503.55 | 2499.48 |
| 1647 | Y3-H-Gtp-H-K(K(LinA)2)-H-Gtp-H-Y3 | 165.6 ± 3.2 | 0.28 ± 0.01 | 12.1 ± 1.3 | 3049.79 | 3045.06 |
| 1649 | Y3-H-Htp-H-K(K(LinA)2)-H-Htp-H-Y3 | 178.5 ± 3.2 | 0.25 ± 0.02 | 11.9 ± 0.3 | 3129.86 | 3126.54 |
| 1650 | Y3-H-Ptp-H-K(K(LinA)2)-H-Ptp-H-Y3 | 180.3 ± 6.1 | 0.38 ± 0.03 | 10.5 ± 2.1 | 3117.76 | 3116.60 |
| 1651 | Y3-H-Ntp-H-K(K(LinA)2)-H-Ntp-H-Y3 | 178.5 ± 3.2 | 0.35 ± 0.05 | 10.9 ± 1.6 | 3217.79 | 3217.51 |
| 1652 | Y3-H-Gtt-H-K(K(LinA)2)-H-Gtt-H-Y3 | 180.5 ± 1.2 | 0.28 ± 0.02 | 11.6 ± 1.3 | 2963.71 | 2960.66 |
| 1653 | Y3-H-GEIPA-H-K(K(LinA)2)-H-GEIPA-H-Y3 | 179.3 ± 2.3 | 0.25 ± 0.01 | 13.1 ± 3.2 | 3019.77 | 3016.79 |
| 1741 | Y3-H-chGtp-H-K(K(LinA)2)-H-chGtp-H-Y3 | 175.4 ± 3.6 | 0.31 ± 0.02 | 11.8 ± 1.3 | 3185.92 | 3176.30 |
| 1742 | Y3-H-dGtp-H-K(K(LinA)2)-H-dGtp-H-Y3 | 169.6 ± 3.9 | 0.22 ± 0.01 | 13.2 ± 1.4 | 3053.75 | 3048.79 |
| 1743 | Y3-H-(TFE-IDAtp)-H-K(K(LinA)2)-H-(TFE-IDAtp)-H-Y3 | 185.6 ± 4.7 | 0.25 ± 0.03 | 12.8 ± 0.6 | 3215.79 | 3209.87 |
| 1744 | Y3-H-IDAtp-H-K(K(LinA)2)-H-IDAtp-H-Y3 | 189.1 ± 4.2 | 0.19 ± 0.02 | 13.7 ± 1.2 | 3051.78 | 3050.72 |
| 1631 | C-Y3-Gtp2-K(K(OHSteA)2)-Gtp2-Y3-C | 168.3 ± 3.9 | 0.29 ± 0.02 | 16.0 ± 2.0 | 3318.06 | 3316.35 |
| 1633 | C-Y3-Htp2-K(K(OHSteA)2)-Htp2-Y3-C | 183.6 ± 4.5 | 0.36 ± 0.01 | 15.4 ± 1.2 | 3478.19 | 3477.14 |
| 1634 | C-Y3-Ptp2-K(K(OHSteA)2)-Ptp2-Y3-C | 190.3 ± 5.1 | 0.33 ± 0.02 | 14.1 ± 3.6 | 3454.00 | 3456.11 |
| 1635 | C-Y3-Ntp2-K(K(OHSteA)2)-Ntp2-Y3-C | 167.2 ± 3.0 | 0.29 ± 0.02 | 14.8 ± 2.8 | 3654.06 | 3650.01 |
| 1636 | C-Y3-Gtt2-K(K(OHSteA)2)-Gtt2-Y3-C | 189.8 ± 9.1 | 0.34 ± 0.05 | 13.2 ± 1.8 | 3145.89 | 3139.57 |
| 1637 | C-Y3-GEIPA2-K(K(OHSteA)2)-GEIPA2-Y3-C | 179.8 ± 5.3 | 0.28 ± 0.02 | 13.4 ± 2.1 | 3258.02 | 3252.99 |
| 1733 | C-Y3-chGtp2-K(K(OHSteA)2)-chGtp2-Y3-C | 185.6 ± 6.6 | 0.26 ± 0.03 | 15.9 ± 1.1 | 3590.31 | 3592.24 |
| 1734 | C-Y3-dGtp2-K(K(OHSteA)2)-dGtp2-Y3-C | 175.5 ± 3.6 | 0.23 ± 0.03 | 16.3 ± 1.3 | 3325.98 | 3320.31 |
| 1735 | C-Y3-(TFE-IDAtp)2-K(K(OHSteA)2)-(TFE-IDAtp)2-Y3-C | 173.3 ± 4.1 | 0.28 ± 0.03 | 15.5 ± 2.1 | 3650.06 | 3650.01 |
| 1736 | C-Y3-IDAtp2-K(K(OHSteA)2)-IDAtp2-Y3-C | 175.1 ± 2.2 | 0.31 ± 0.02 | 17.2 ± 1.6 | 3322.04 | 3318.22 |

*All sequences were synthesized and analysed with an N-terminal azido lysine (K(N₃).

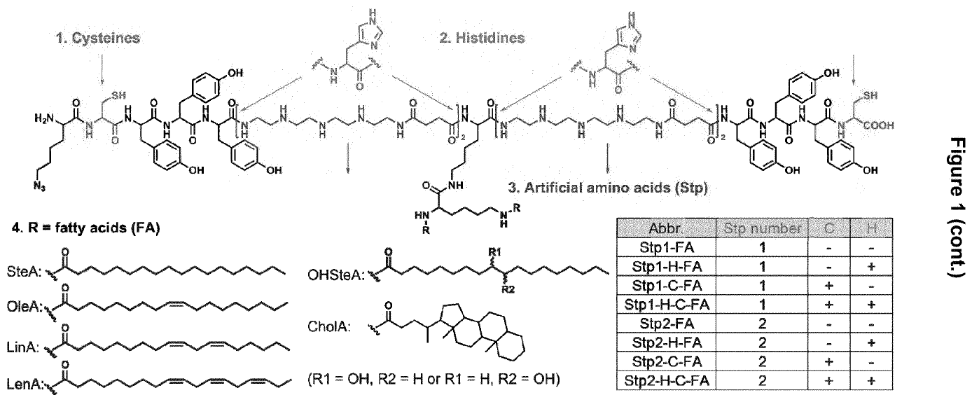

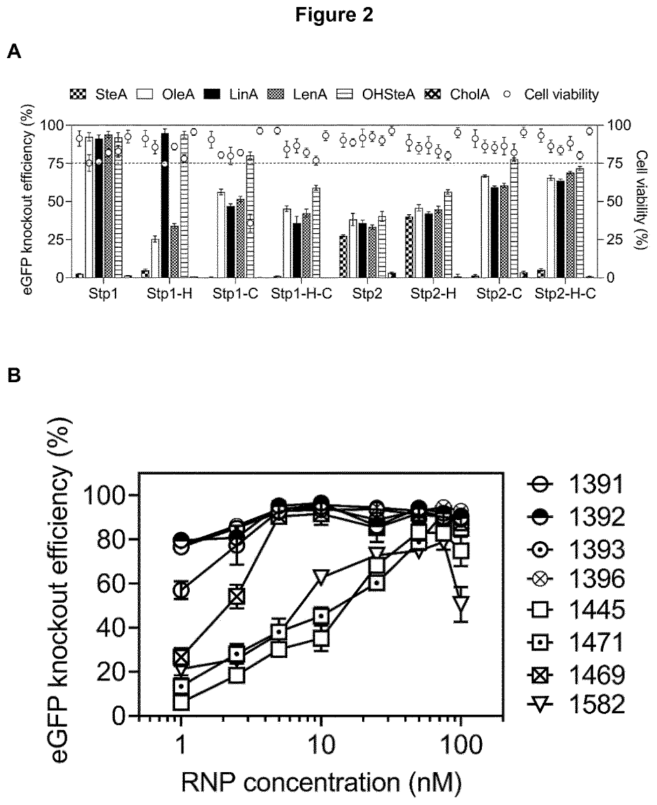

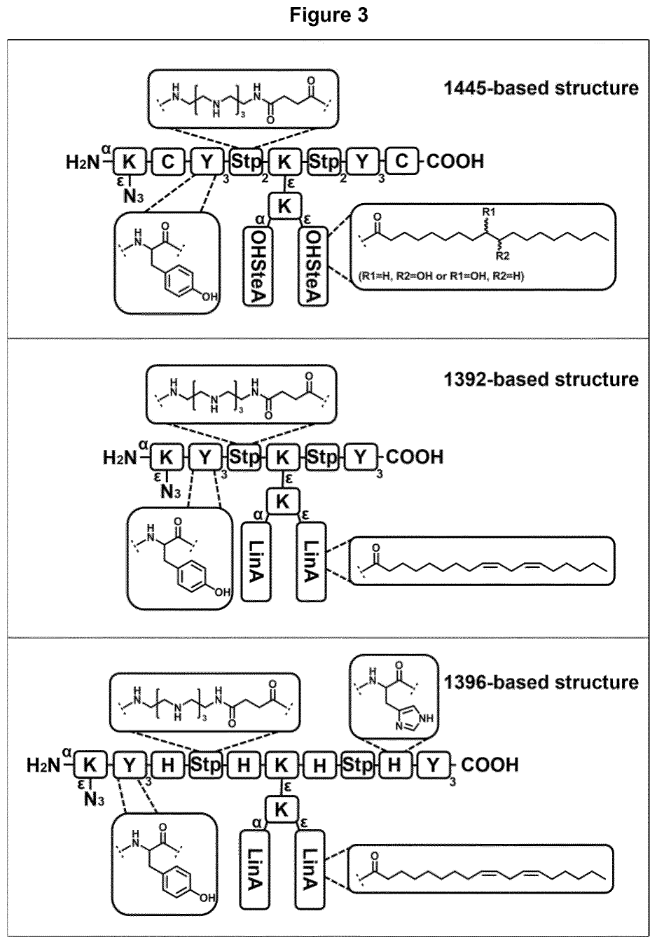

The gene knockout efficiency and cytotoxicity of the nanoparticles were investigated in HeLa eGFP/tub cells after treatment with 75 nM RNP for 48 h (FIG. 2A). The systematic evaluation demonstrated the effect of each variation. Firstly, cysteine showed benefits in lipo-oligomers containing 2 Stp units at each side beyond the lysine (Stp2 structures), but rather decreased the transfection efficiency in Stp1-based sequences. Secondly, histidine which has been previously shown to facilitate endosomal escape of pDNA polyplexes did not induce significant improvement in case of RNP nanoparticles. Thirdly, Stp1 structures in general demonstrated a better knockout performance than Stp2 structures. Lastly, in all cases unsaturated (OleA, LinA, LenA) or hydroxyl-modified fatty acids (OHSteA) were essential to mediate significant gene editing; saturated stearic acid (SteA) as well as the steroid cholanic acid almost completely interrupted any knockout efficiency. Seven lipopeptides exhibited better or comparable knockout efficiency than the initial structure with the oligomer ID #1445 (Stp2-C-OHSteA) and especially the Stp1 series outperformed the preceding lead structure. Over 90% of eGFP knockout was achieved with these structures at the dose of 75 nM RNP. To differentiate the potency of the nanoparticles more in depth, a dose titration experiment was conducted in the concentration range of 1 nM-100 nM RNP (FIG. 2B). The lead structure Stp2-C-OHSteA (#1445, FIG. 3 top) showed a clear dose-dependent activity and eGFP knockout levels dropped below 40% at concentrations of 10 nM RNP or lower.

The new lipopeptide analogs Stp1-LinA (#1392, FIG. 3 middle), Stp1-LenA (#1393) and Stp1-H-LinA (#1396, FIG. 3 bottom) turned out to be much more potent and induced ~80% eGFP knockout even at the lowest RNP dose of 1 nM. Cell viability studies showed that all nanoparticles were generally well-tolerated except Stp1-C-OHSteA (data not shown).

### Example 2: Oligomers with Optimized Artificial Amino Acid Building Blocks

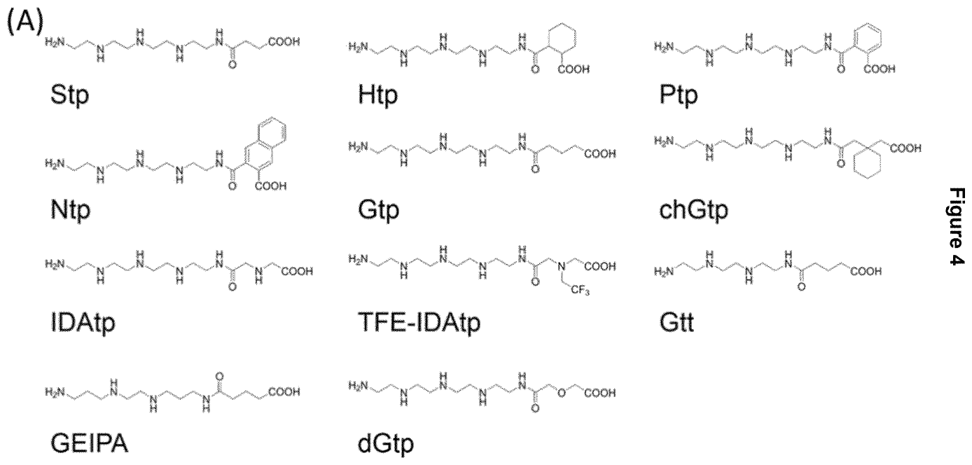

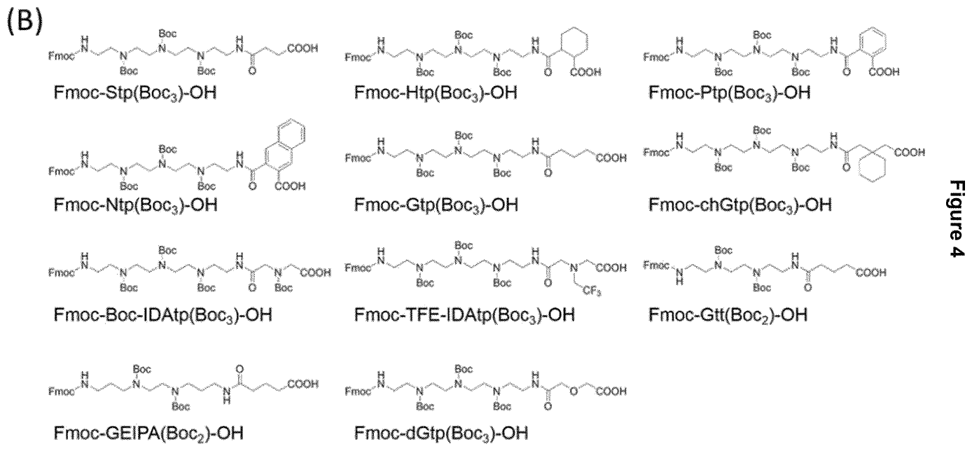

Due to the generally more hydrophobic nature of the identified outperformers with lower content of ionizable artificial amino acid than the initial Stp2-C-OHSteA (#1445), it was hypothesized that the hydrophobicity of the lipo-oligomers may be an important parameter to achieve efficient Cas9 RNP delivery. Consequently, the potency could be improved even further by fine tuning the hydrophobic properties of the artificial amino acid units. To validate this hypothesis, a series of artificial amino acid building blocks with varied hydrophilic or hydrophobic structural elements and suitable protective groups for SPPS was synthesized (Scheme 1 and 2 above, FIG. 4B).

Eight more hydrophobic Stp analogs were obtained by replacing tetraethylenepentamine (tp) by oligoamines with lower number of ionizable nitrogens (triethylenetetramine, tt or 3,3′-ethylenediiminodipropylamine, EIPA), and by including more hydrophobic dicarboxylic acids instead of succinic acid. Two more hydrophilic analogs were generated by replacing succinic acid by more hydrophilic dicarboxylic acids containing an amine or ether group (IDAtp and dGtp). For a systematic evaluation of the new building blocks, the backbone sequences of the two best-performers Stp1-LinA (#1392) and Stp1-H-LinA (#1396), as well as the initial lead structure Stp2-C-OHSteA (#1445) were selected as lipo-oligomer architectures (FIG. 3). The combination of 11 building blocks with 3 architectures resulted in a total of 33 lipopeptides which were synthesized by SPPS (#1445, #1392, #1396 and #1631- #1744 of Table 1).

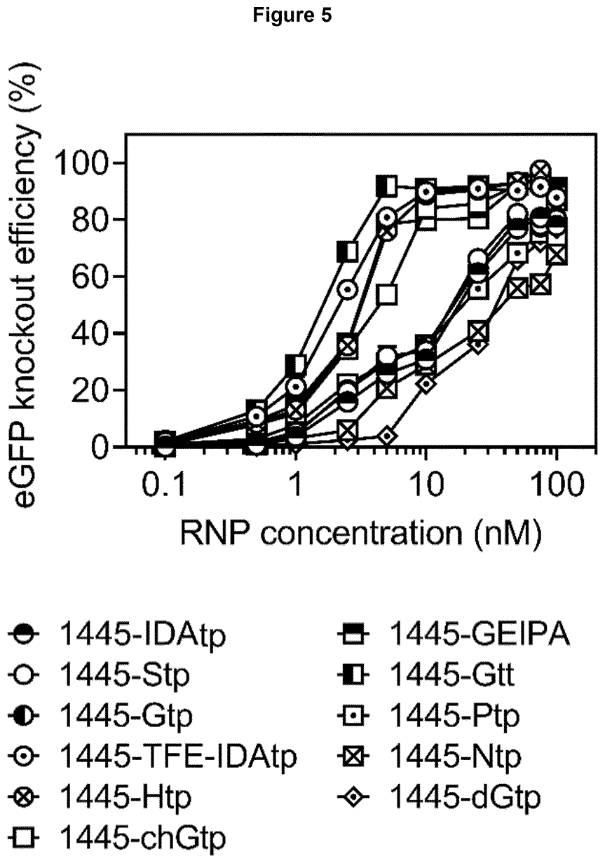

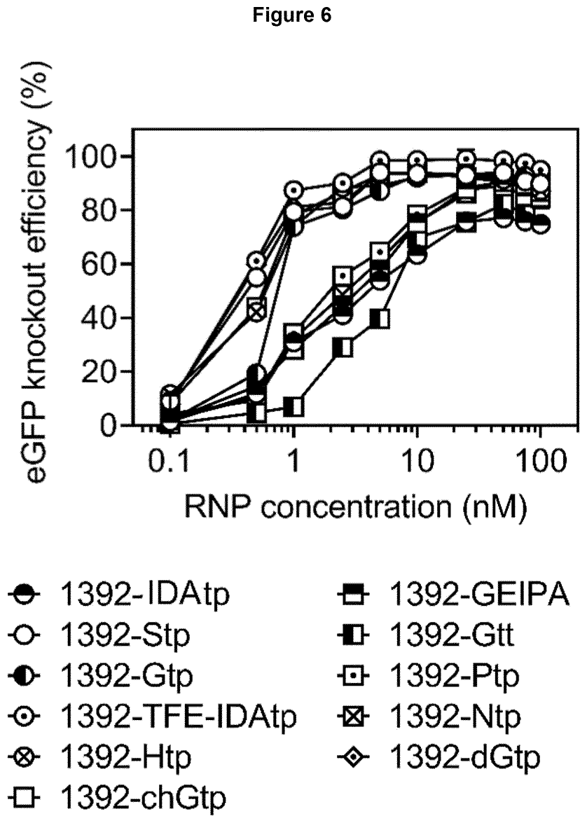

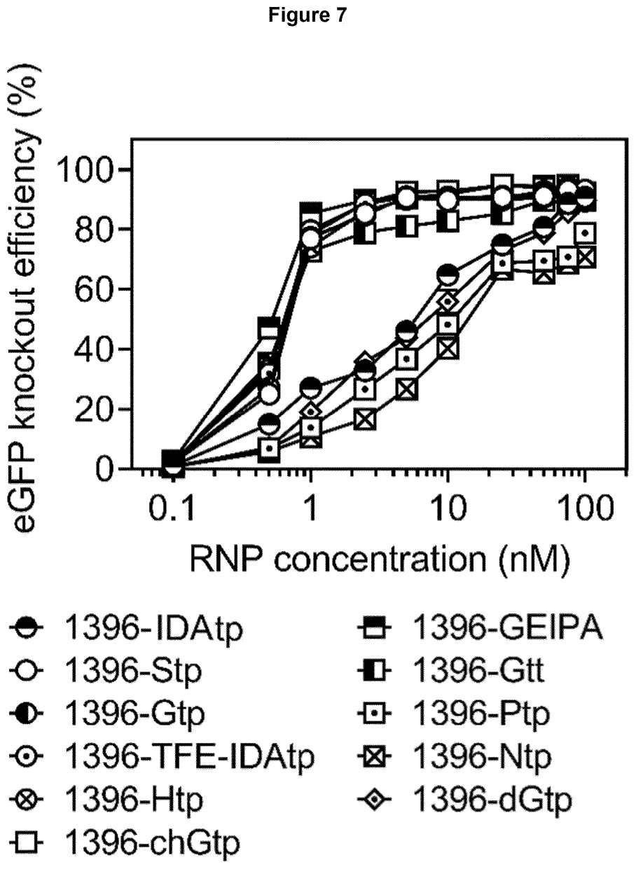

LogD7.4 of each lipopeptide was determined by quantifying the compound concentration in octanol and water phases by reversed-phase HPLC after mixing and phase separation (H. J. Kim et al., ACS Central Science 2019, 5, 1866-1875; A. Andrés et al., European Journal of Pharmaceutical Sciences 2015, 76, 181-191; C. Giaginis and A. Tsantili-Kakoulidou, Journal of Liquid Chromatography & Related Technologies 2007, 31, 79-96). The determined logD7.4 values (data not shown) indicated the following order of artificial amino acids with increasing hydrophobicity: IDAtp <dGtp <Stp <Gtp <TFE-IDAtp <Htp <chGtp <Gtt <GEIPA <Ptp <Ntp. Cas9 RNP nanoparticles were then formulated with the lipopeptides (N/P=24), and the size, PDI and zeta potential were determined by DLS. All lipopeptides formed homogeneous nanoparticles with Cas9 RNP with a size of 140-190 nm, PDI of 0.15-0.38, and zeta potential of 10.5-17.2 mV (Table 2). A dose titration study in the RNP concentration range between 0.1 nM-100 nM was performed and the eGFP knockout EC50 of each lipopeptide was calculated (FIG. 5-7).

In general, structures based on the X¹-LinA architecture (1392-based structures) exhibited the best knockout efficiency, where 6 out of 11 lipo-oligomers achieved ~75-80% eGFP knockout at 1 nM RNP dose and 3 out of 11 lipopeptides even induced over 50% eGFP disruption at 0.5 nM. X¹-H-LinA based structures (1396-based structures) also demonstrated very high potency, but slightly lower efficacy than X¹-LinA derivatives (1392-based structures) at very low doses of RNP (0.5 nM). Notably, the performance of X²-C-OHSteA structures (1445-based structures) was significantly enhanced by introducing more hydrophobic artificial amino acids (Gtt, GEIPA, Htp, chGtp, TFE-IDAtp). The best performers of each lipopeptide series were identified as Gtt₂-C-OHSteA (#1636), GEIPA1-H-LinA (#1653), and TFE-IDAtp1-LinA (#1739). Especially, TFE-IDAtp1-LinA (#1739) achieved up to 99% eGFP knockout at RNP concentrations down to 5 nM, ~88% knockout at 1 nM, and still enabled ~61% eGFP disruption at 0.5 nM. Cell viability studies revealed that Htp and chGtp containing structures were toxic at high RNP concentrations (25-100 nM) while all other artificial amino acids-containing lipopeptides were well-tolerated in the investigated concentration range (data not shown).

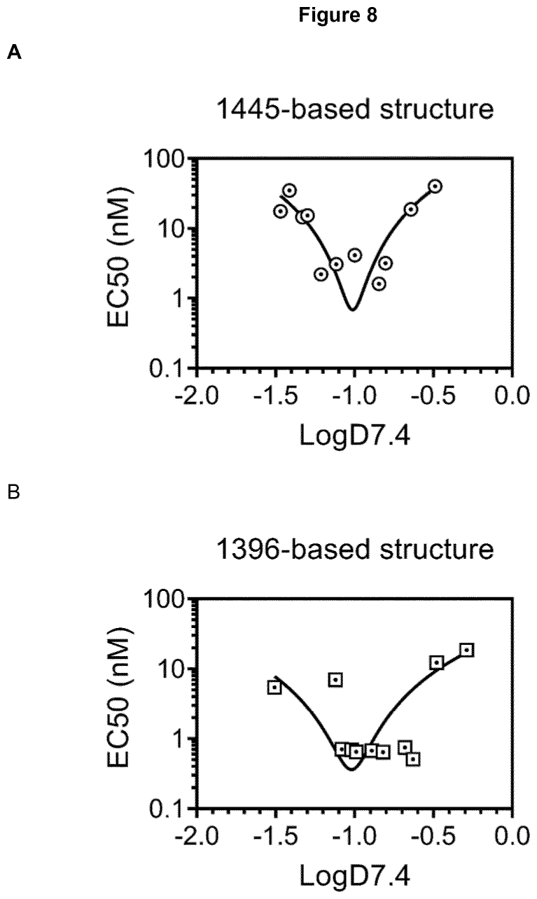

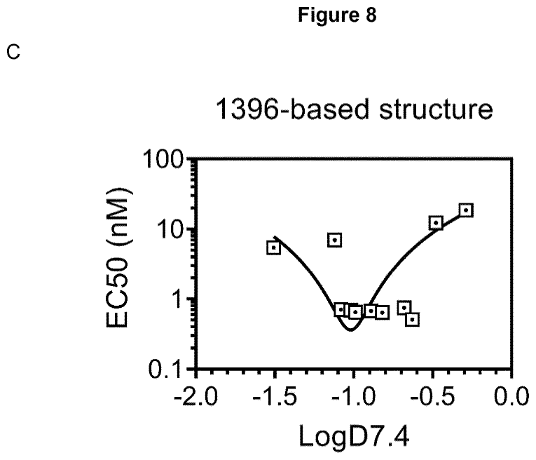

To correlate the obtained structure-activity relationships with hydrophobic characteristics, the logD7.4 was plotted versus eGFP knockout EC50 values for each lipopeptide series (FIG. 8). A clear correlation between the logD7.4 and EC50 was found, and optimal logD7.4 ranges were identified for each sequence-defined lipopeptide series. The efficacy of structures with logD7.4 values beyond the optimal range (too hydrophilic or too hydrophobic) dramatically dropped.

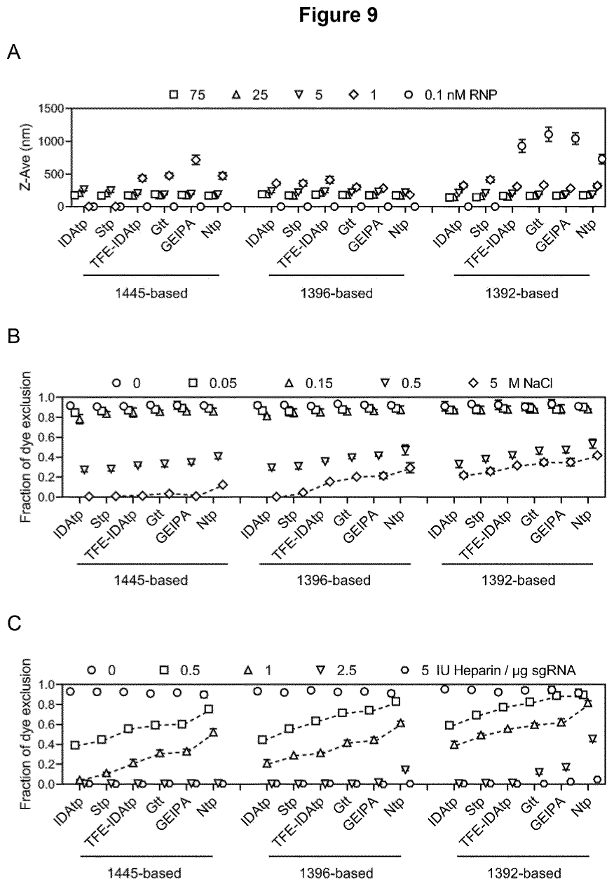

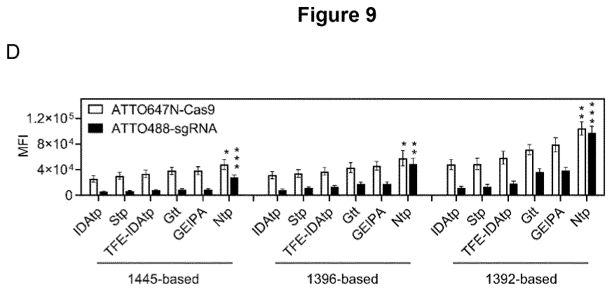

To explore the reason why hydrophobically balanced lipopeptides are more potent, especially at low RNP concentrations, a group of representatives covering the spectrum from most hydrophilic to most hydrophobic were selected for investigation of the impact on critical carrier characteristics. The set of lipo-oligomers consisted of IDAtp-, Stp-, TFE-IDAtp-, Gtt-, GEIPA-, and Ntp-based structures. Firstly, the integrity of the nanoparticles upon dilution was investigated (FIG. 9A). The nanoparticles were prepared at a high RNP concentration of 375 nM and then sequentially diluted to concentrations ranging from 0.1 nM to 75 nM. The size of IDAtp2-C-OHSteA (#1736) and Stp2-C-OHSteA (#1445), the two most hydrophilic X²-C-OHSteA structures (1445-based structure), immediately increased upon dilution and was not detectable at a RNP concentration of 1 nM or lower.

In contrast, the more hydrophobic analogs exhibited improved dilution stability. A similar tendency was observed with the more hydrophobic X¹-H-LinA (1396-based structure) and X¹-LinA (1392-based structure) architectures that were generally more resistant towards dilution. Nanoparticles generated with the most hydrophobic structure, Ntp1-LinA (#1343), retained a stable size between 180-220 nm at 2.5-375 nM of RNP and were still detectable at 0.1 nM. In addition, the stability of the nanoparticles against excessive amounts of ions was investigated by the detection of RNA release with the intercalating dye Ribogreen (FIGS. 9B and 9C). Sodium chloride (NaCl) and the polyanion, heparin, were selected as ionic stress factors in this study. At isotonic NaCl concentration (0.15 M), all the nanoparticles showed robust encapsulation of Cas9 RNP. With increasing NaCl concentrations, higher Ribogreen fluorescence was detected indicating lower dye exclusion and therefore release of Cas9 RNP from dissociated nanoparticles (FIG. 9B). Notably, the more hydrophobic lipopeptides of each series exhibited better resistance against NaCl. Similar trends were found in the heparin competition assay, where hydrophobic artificial amino acids remarkably enhanced the nanoparticle stability against polyanionic stress (FIG. 9C).

To examine the cellular uptake of hydrophobically balanced Cas9 RNP nanocarriers, flow cytometry experiments were performed with ATTO647N-labeled Cas9 and ATTO488-labeled sgRNA (FIG. 9D). The results demonstrated a general correlation of higher cellular uptake with increasing hydrophobic properties. A representative example are the hydrophobic Ntp-based structures, where Ntp2-C-OHSteA (#1635) and Ntp1-LinA (#1643) achieved 4.4- and 7.3-foldenhanced sgRNA uptake compared to the Stp-based analogs. CLSM studies further confirmed the observation that Ntp2-C-OHSteA (#1635) and Ntp1-LinA (#1643) leads to higher Cas9 RNP uptake, indicated by the brighter intracellular fluorescence in HeLa cells (data not shown). Furthermore, the endocytosis pathways of Stp2-C-OHSteA (#1445), Ntp2-C-OHSteA (#1635), Stp1-LinA (#1392), and Ntp1-LinA (#1643) nanoparticles were probed by pretreating the cells with different inhibitors or incubation at 4° C. (data not shown). Low temperature and sodium azide inhibited the uptake of all 4 nanoparticles by 85-90% and 65-77% respectively, indicating an energy-dependent internalization mechanism. Chlorpromazine and sucrose were the only other inhibitors that significantly reduced the uptake of the more hydrophilic Stp2-C-OHSteA (#1445) nanoparticle, suggesting a dominant role of clathrin-mediated endocytosis. Interestingly, the Cas9 RNP uptake mediated by the more hydrophobic lipopeptides Ntp2-C-OHSteA (#1635), Stp1-LinA (#1392), and Ntp1-LinA (#1643) could also be blocked by pretreatment with amiloride, which indicates a contribution of macropinocytosis in addition to the clathrin-mediated pathway. Notably, macropinosomes are reported to be more leaky than other endosomes, which may be beneficial for the translocation of nanoparticles into the cytosol (J. S. Wadia et al., Nature Medicine 2004, 10, 310-315; Z. Zhang et al., Advanced Materials 2021, 33, 2102219; W. Zhang et al., Theranostics 2019, 9, 1580-1598). Following the intracellular delivery pathway stepwise, an endosomal escape reporter cell line HeLa mRuby3/gal8 (Y. Rui et al., Science Advances 2022, 8, eabk2855) was used to evaluate the endosomal escape capability of the selected nanoparticles (data not shown). In this model, the rupture of endosomal membranes results in recruitment of a mRuby3/galectin-8 (gal8) fusion protein which is visible by intracellular punctate red spots. After 4 h treatment, the Ntp-based structures were found to induce a higher number of endosomolytic events than their Stp counterparts, especially at a low RNP concentration (5 nM). Notably, the peptide architecture played an additional fundamental role in the endosomal escape process, since the X¹-LinA sequences (1392-based structures) showed much better endosomal escape efficiency than the X²-C-OHSteA lipo-oligomer (1445-based structure).

Taken the above results together, the mechanistic effects can be explained as follows: hydrophobic lipopeptides form Cas9 RNP nanoparticles, which 1) are more resistant towards dilution-mediated dissociation and remain intact at low concentrations; 2) are more resistant towards ionic stress and avoid premature cargo release; 3) mediate more efficient cellular internalization that is driven by both clathrin-mediated endocytosis and macropinocytosis; and 4) possess a higher endosomal escape capacity. The fact, that optimal logD7.4 ranges and a requirement of finding the right balance have been observed can be explained, since too stable nanoparticles may result in insufficient cargo release in the right place at the right time.

### Example 3

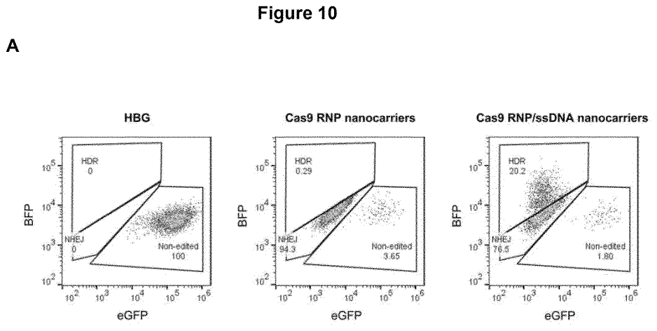

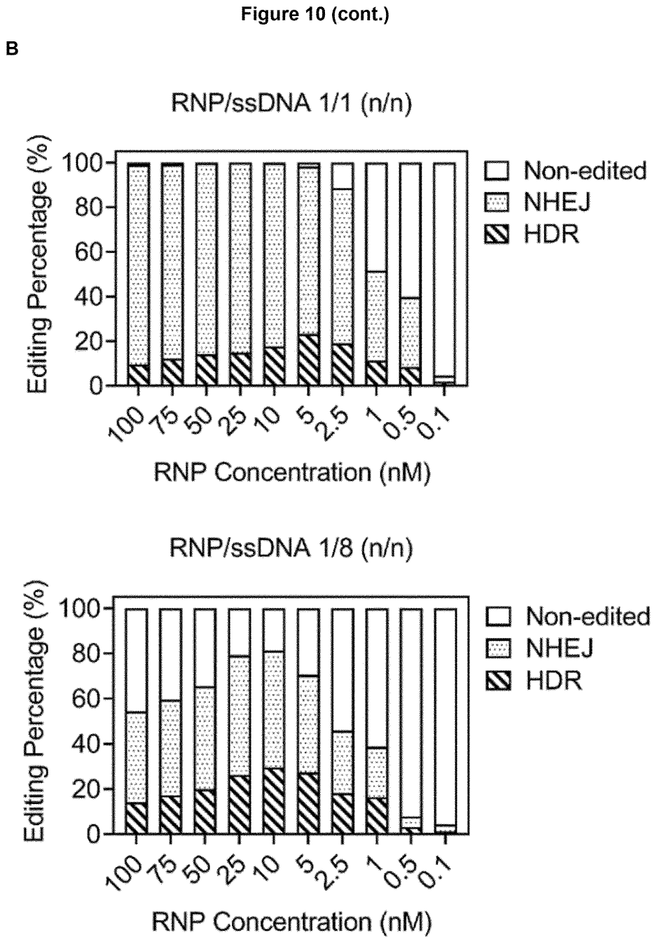

Having demonstrated that hydrophobically balanced lipopeptides are potent nanoparticles for delivering Cas9 RNP to mediate gene knockouts via non-homologous end joining (NHEJ), we sought to extend the scope of application: the top 5 lipopeptides of each series were selected for the co-delivery of Cas9 RNP and a ssDNA template to mediate gene knockins via homology directed repair (HDR). A HeLa cell line expressing destabilized eGFP (HeLa GFPd2) (Rui et al., ACS Appl Mater Interfaces, 11 (11), 2019, 10472-10480) was used in this study. The plasmid PB-CAG-GFPd2, which was used for the generation of HeLa GFPd2 was a gift from Jordan Green (Addgene plasmid #115665; http://n2t.net/addgene: 115665; RRID: Addgene_115665). The 66th amino acid in the eGFP sequence, tyrosine (code: TAC), can be replaced by histidine (code: CAT) via HDR-mediated DNA repair, which results in the conversion of eGFP into BFP (L. Farbiak et al., Advanced Materials 2021, 33, 2006619; R. Xie et al., Advanced Materials 2022, 34, 2110618; J. Walther et al., Pharmaceutics 2022, 14). Three cell populations can be expected after such treatments: 1) eGFP positive cells, representing non-edited cells; 2) eGFP and BFP negative cells representing cells with NHEJ-mediated gene knockout; and 3) BFP positive and eGFP negative cells representing HDR-mediated gene corrected cells (FIG. 10A). In the first step, TFE-IDAtp1-LinA (#1740), which performed best in gene knockout experiments, was used for finding the most suitable RNP/ssDNA ratios. The nanoparticles for HDR were prepared by complexing TFE-IDAtp1-LinA (#1740) with different RNP/ssDNA ratios ranging from I/O.5 to 1/20 at a N/P ratio of 12 (representative ratios of RNP/ssDNA 1/1 and ⅛ are shown in FIG. 10B). HeLa GFPd2 cells were treated with the Cas9 RNP/ssDNA nanoparticles for 48 h followed by cell population analysis via flow cytometry. As shown in FIG. 10, the best knockout and total editing efficiencies were achieved at high RNP concentrations (10-100 nM) and high RNP/ssDNA ratios (I/O.5-1/3, shown in 1/1), while the highest HDR levels were induced at moderate to low RNP concentrations (10-1 nM) and moderate RNP/ssDNA ratios (1/3-1/8, shown is 1/8). At

RNP/ssDNA ratios below 1/10, almost all editing events were blocked, presumably due to severe cytotoxicity (data not shown). The highest HDR efficiency of ~28% was achieved at a dose of 10 nM RNP and 1/8 RNP/ssDNA ratio (FIG. 10B, bottom). Notably, a general trend of increasing percentage of HDR events in relation to total editing was observed with decreasing RNP concentration, which is explainable by the competition between NHEJ and HDR repair pathways.

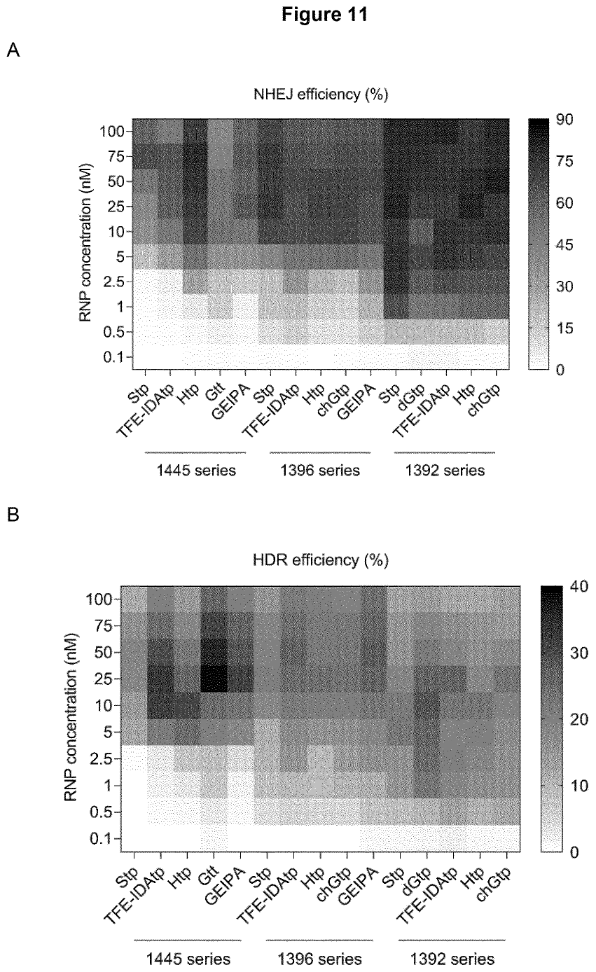

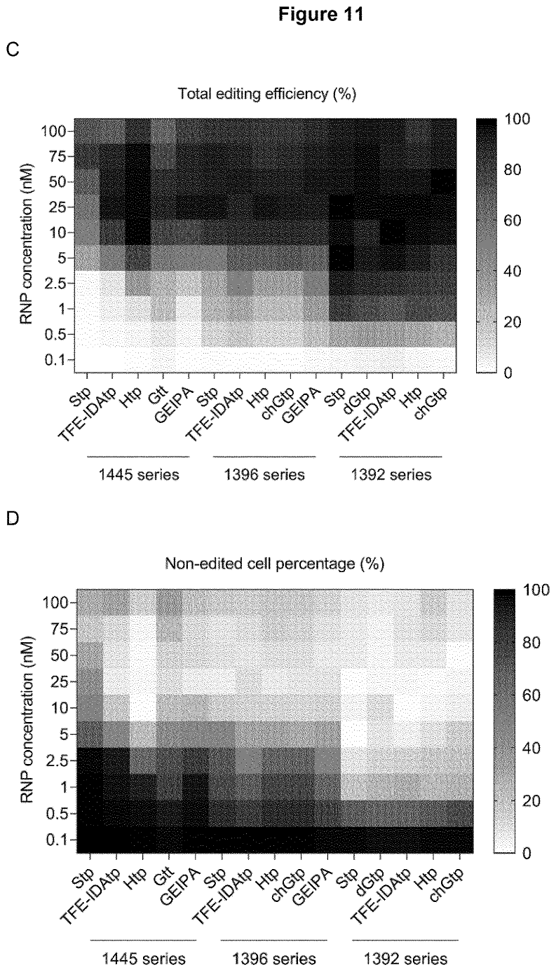

Next, the NHEJ efficiency (FIG. 11A), HDR induction efficiency (FIG. 11B) and total editing efficiency (FIG. 11C) of the other selected lipopeptides was investigated at a RNP/ssDNA ratio of 1/4 to circumvent cytotoxicity. Surprisingly, the X²-C-OHSteA series (1445-based structures) that was the least efficient in gene knockout experiments, achieved the highest maximum HDR levels (FIG. 11B). Especially the hydrophobically balanced Gtt₂-C-OHSteA (#1636), enabled HDR induction up to 40% at a 25 nM RNP dose. Nevertheless, X¹-LinA lipo-oligomers (1392-bases structures) still showed the highest potency and best HDR efficiency at low concentrations (FIG. 11B). For instance, TFE-IDAtp1-LinA nanoparticles (#1739) induced ~23% and ~11% HDR in HeLa GFPd2 cells treated with 1 nM or 0.5 nM RNP doses, respectively. Consistent with the studies in HeLa GFP/tub cells, lipo-oligomers with X¹-LinA architecture (1392-based structures) achieved the highest and most potent GFP knockout via NHEJ in HeLa GFPd2 cells (FIG. 11A). The total editing efficiency (FIG. 11C), which refers to the union of NHEJ and HDR edits, confirms the advantage of the X¹-LinA architecture (1392-based structures), which achieved high maximal editing levels and demonstrated the highest potency at low concentrations.

In summary, a series of artificial amino acids and derived lipo-oligomers for the intracellular delivery of Cas9 RNP were designed and synthesized. Systematic biological evaluation revealed that the hydrophobic characteristics play a decisive role for potent gene editing, especially at low concentrations. The fluorinated lipopeptide TFE-IDAtp1-LinA (#1739) was found to be a particularly potent nanocarrier which can achieve 88% NHEJ gene knockout and 23% HDR knockin at just 1 nM RNP concentration. The identified relationships between logD7.4 values, carrier characteristics and impact on cellular delivery are suggested to serve as a guide for the future design of Cas9 RNP nanocarriers. The new artificial amino acids and lipopeptide architectures provide a versatile platform for the creation of highly potent and tunable cellular delivery agents.

### Example 4

The present sequence-defined T-shape oligomers were also tested for delivery of other cargo, such as PMO.

In an initial experiment sequence-defined T-shape oligomers comprising the sequence of formula | (#1392 (Stp), #1639 (Gtp), #1641 (Htp), #1643 (Ntp), #1644 (Gtt), #1645 (GEIPA), #1737 (chGtp), #1738 (dGtp), #1739 (TFEIDAtp), #1740 (IDAtp)) and with histidine (#1395 (Stp), #1396 (Stp), #1647 (Gtp), #1649 (Htp), #1651 (Ntp), #1652 (Gtt), #1653 (GEIPA), #1741 (chGtp), #1742 (dGtp), #1743 (TFEIDAtp), #1744 (IDAtp)) were tested for PMO delivery by covalently binding the PMO to the oligomers at a ratio of 1:3 and determining the bioactivity in HeLa mCherry-DMDEx23 reporter cells as described in Example 5. The reporter cell line enables determination of PMO-mediated modification of mRNA splicing via the induction of mCherry expression. These oligomers comprising the sequence of formula I were more effective compared to oligomer #1195 (according to formula III; Y₃-Stp2-K (G-K (LenA) 2-Stp2-Y₃; SEQ ID NO: 62 comprising an N-terminal azido-lysine and a glycine between K_{c} and Kᵣ) by several orders of magnitude when tested at a range from 2.4 nM to 625 nM of PMO (data not shown). PMO-1395 resulted in 100% mCherry positive cells at 625 nM while PMO-1195 only showed <10% positive cells at the same concentration. A similar result was achieved with 9.8 nM PMO-1395. At PMO concentrations of 6.25 nM, 312.5 nM and 625 nM, the oligomers comprising Stp, chGtp, dGtp or TFEIDAtp as artificial amino acid showed best results out of the tested compounds, particularly TFEIDAtp. Oligomers comprising OleA (#1395) were as effective as the same construct comprising LinA (#1396).

### Example 5

In another experiment further sequence-defined T-shape oligomers were tested for PMO delivery.

### Preparation of PMO-Lipo-Oligomer Formation

PMO functionalization with DBCO-NHS ester and purification was performed as described in Kuhn et al., (Adv. Funct. Mater. 2019, 29, 1906432). For the conjugation of PMO-DBCO and lipo-oligomer at a molar ratio of 1:3-PMO to lipo-oligomer (LP), a dilution of PMO-DBCO with the concentration of 100 UM in water and a dilution of the lipo-oligomer with the concentration of 300 UM in water were prepared. Equal volumes of the two solutions were combined and incubated overnight at room temperature (RT) while shaking at 300 rpm. The resulting PMO-LP conjugate solution has a concentration of 50 UM PMO. The formulation solution was freeze-dried and reconstituted with the required volume of HBG (20 mM HEPES, 5% glucose, pH 7.4) to obtain the desired concentration.

Splice-Switching Activity of PMO-LP Formulations in HeLa mCherry DMDEx23 Reporter Cells

The splice-switching activity of PMO-LP formulations was investigated in HeLa mCherry-DMDEx23 reporter cells which express mCherry upon specific oligonucleotide mediated splicing-modulation. The day before cell treatments, HeLa mCherry-DMDEx23 cells were seeded into 96-well plates (Corning® Costar, Sigma-Aldrich, Munich, Germany) at a density of 5×10³ cells/well. Prior to cell treatments, the medium in each well was replaced by 90 μL medium and 10 μL PMO-LP conjugate formulations was added resulting in the desired PMO concentrations. The cells were incubated at 37° C. and 5% CO₂ in a humidified atmosphere. 24 h after transfection, the medium was removed and the cells were washed with 100 μL PBS. The cells were trypsinized and resuspended in 100 μL FACS buffer, consisting of 10% FBS in PBS substituted with DAPI (1 ng/μL) to differentiate between live and dead cells. All samples were analyzed by flow cytometry using a CytoFLEX S flow cytometer (Beckman Coulter, Carlsbad, USA). The fluorescence of the cells was determined by measuring excitation of DAPI at 405 nm and detection of emission at 450 nm, excitation of mCherry at 561 nm and detection of emission at 610 nm. Only isolated and viable cells were evaluated. Flow cytometry data was analyzed using FlowJo X 10.0.7r2 flow cytometric analysis software by FlowJo, LLC (Ashland, USA). All experiments were performed in triplicates.

### Cell Metabolic Activity Assay

Cell viability was determined via investigation of cell metabolic activity with MTT assay. One day prior to cell treatments, HeLa mCherry-DMDEx23 were seeded in 96-well plates at a density of 5 ×10³ cells/well. 24 h after seeding, the medium was replaced by 90 μL of fresh medium. 10 μL PMO-LP formulation were added to each well resulting in the desired PMO concentrations. After incubation for 24 h at 37° C. and 5% CO₂ in humidified atmosphere, 10 μL of MTT (3-(4,5-dimethylthiazol-2-yl)-2,5-diphenyltetrazolium bromide) (5 mg/mL) was added to each well. After 2 h of incubation, medium containing unreacted dye was removed, and the 96-well plates were stored at −80° C. overnight. 100 μL DMSO was added per well to dissolve the purple formazan product. The 96-well plates were incubated for 30 min at 37° C. while constantly shaking. The absorbance at λ=590 nm of each well was measured with background correction at A=630 nm using a microplate reader (Tecan Spark 10M, Tecan, Nänikon, Switzerland). The relative cell viability (%) was calculated by normalizing the values to control wells treated with HBG. The following equation was used:

$$cell viability [ \% ] = \frac{A ( test )}{A ( control )} \cdot 100 \%$$

A, difference of absorbance at λ=590 nm and background correction at A=630 nm Means are reported+/−standard deviation.

### Results:

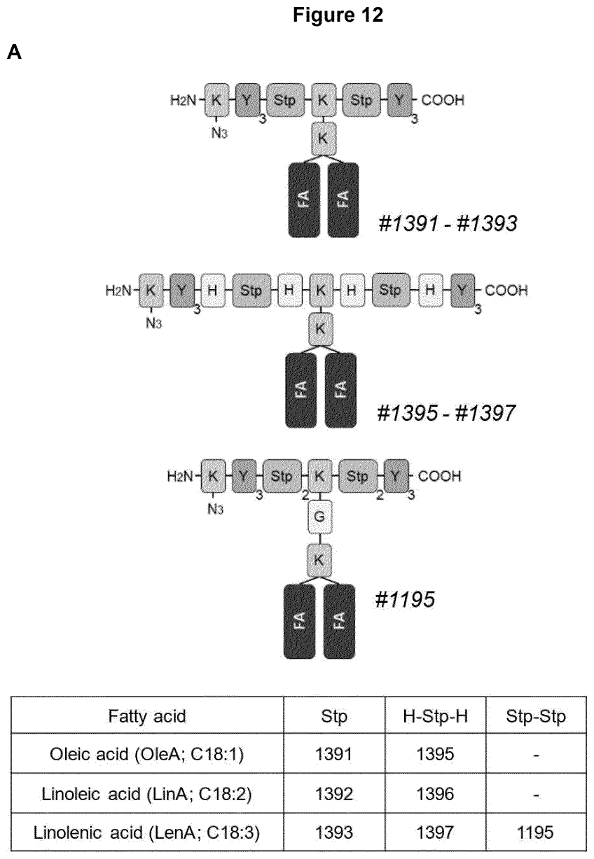

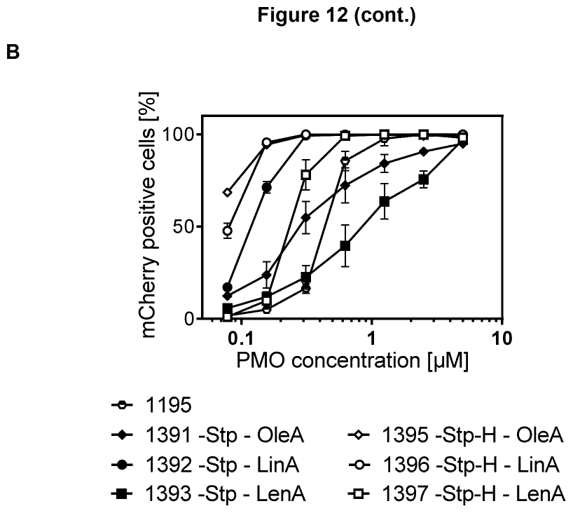

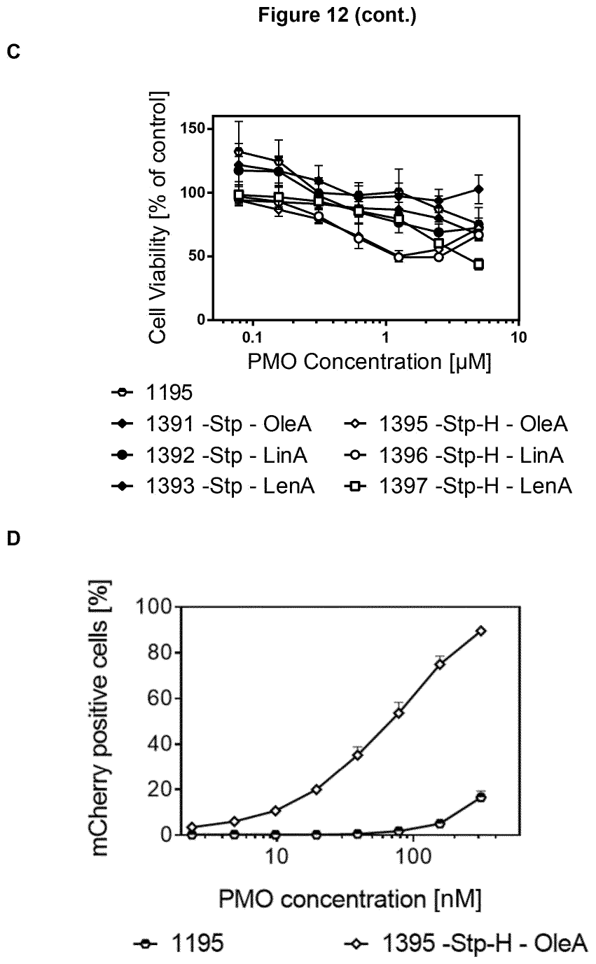

The novel sequence-defined T-shape oligomers #1391, #1392, #1393, #1395, #1396 and #1397, all comprising Stp, were tested in comparison to the more polar reference carrier #1195 (FIG. 12A). In direct comparison five of the tested oligomers exhibited much higher potency (FIG. 12B). Histidine containing oligomers generally mediated higher transfection efficiencies even at low concentrations. Oleic acid and linoleic acid (#1395 and #1396) were found to be favorable structural elements in the new LP architectures (FIG. 12B). Over 90% of cells were mCherry positive after treatment with the most potent oligomers #1395 and #1396 containing 156.25 nM PMO. In all cases, the ratio of mCherry expressing cells increased with increasing PMO concentration. At the same time a dose dependent cytotoxicity was observed (FIG. 12C), which supports the requirement for potent oligomers, which achieve high activity at low concentrations. The best performing PMO formulations were based on LP #1392, #1395, #1396. Especially the oligomers #1395 and #1396 turned out to be very potent and resulted in >45% mCherry positive cells even after exposure of only 5 minutes to the moderate PMO dose of 312.5 nM (data not shown). Transmission electron microscopy (TEM) confirmed self-association of PMO (Ex23)-1395 1:3 formulations into nanoparticles, similar to oligomer #1195 (data not shown). The increased splice switching activity correlated with higher PMO internalization mediated by the #1395 oligomers compared to #1195 oligomers.

To assess the favorable properties of #1395 oligomers in more detail, an additional comparison was carried out at low PMO concentrations between 2.4 nM and 312.5 nM and analysed following 24 h treatment. The dose response curves (FIG. 12D) clearly illustrate the much higher potency of #1395, which mediated splice-switching already at PMO concentrations≤5 nM.

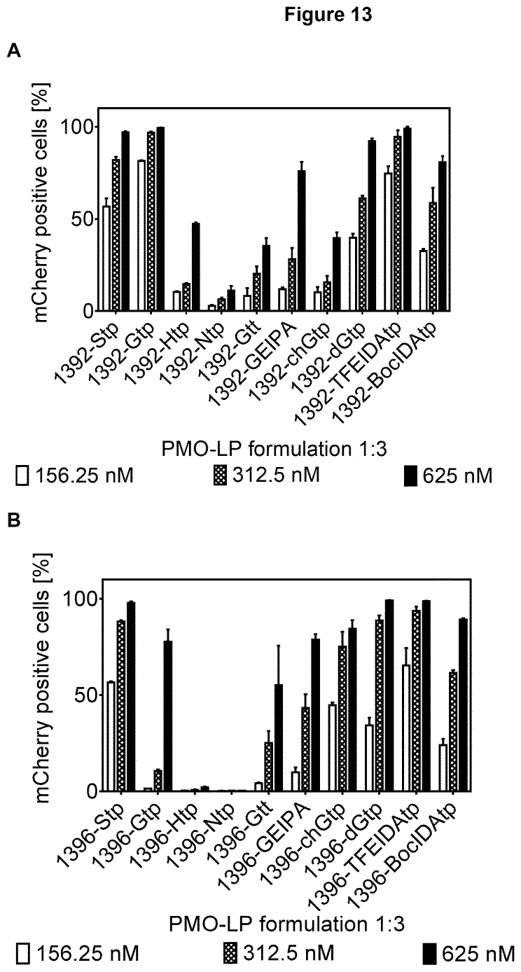

Further #1392 and #1396 analoga were tested. Of the 11 1392-based and 1396-based structures described in Example 2 using different, mainly more hydrophobic, artificial amino acids, 10 (all except 1392-Ptp and 1396-Ptp) were also tested for PMO delivery at three different concentrations (156.25 nM, 312.5 nM, 625 nM) as described above. As may be taken from FIG. 13A 1392-dGtp and 1392-IDAtp showed about comparable or slightly lower activity and 1392-Gtp and 1392-TFE-IDAtp showed better activity compared to 1392-Stp. FIG. 13B shows that (FIG. 13A) for 1396-based structures TFE-IDAtp showed superior activity and dGtp about comparable activity and chGtp and IDAtp slightly lower activity compared to Stp.

### Example 6

### SIRNA Polyplex Formation

siRNA and lipo-oligomer at an indicated N/P ratio (nitrogen/phosphate) were separately diluted in equal volume of HBG (20 mM of HEPES, 5% (w/v) glucose, pH 7.4). Only protonatable nitrogens were considered for N/P ratio calculation. The siRNA and lipo-oligomer solutions were mixed by rapid pipetting and incubated for 45 min at room temperature (RT). The final concentration of siRNA in the polyplex solution was 25 μg/mL.

siRNA duplexes were obtained from Axolabs GmbH (Kulmbach, Germany): eGFP-targeting SiRNA (siGFP) (sense strand: 5′-AuAucAuGGccGAcAAGcAdTsdT-3′, SEQ ID NO: 144; antisense strand: 5′-UGCUUGUCGGCcAUGAuAUdTsdT-3′, SEQ ID NO: 145) for silencing of eGFPLuc; control siRNA (siCtrl) (sense strand: 5′-AuGuAuuGGccuGuAuuAGdTsdT-3′, SEQ ID NO: 146; antisense strand: 5′-CuAAuAcAGGCcAAuAcAUdTsdT-3′, SEQ ID NO: 147); small letters indicate 2′methoxy modifications; “s” indicates phosphorothioate linkages, “dT” refers to deoxythymidine.

Particle Size and Zeta Potential Measurement for siRNA Polyplexes

Particle size and zeta potential were measured in a folded capillary cell (DTS1070) with dynamic and electrophoretic laser-light scattering (DLS, ELS) using a Zetasizer Nano ZS (Malvern Instruments, Malvern, Worcestershire, U.K.). siRNA polyplexes were prepared in a total volume of 80 μL HBG with siRNA concentration of 25 μg/mL. Size and polydispersity index (PDI) were measured after an equilibration time of 30 sec at 25° C. with refractive index of 1.330 and viscosity of 0.8872 mPa*s. Samples were measured three times with six sub runs per measurement. For zeta potential measurement, each sample was diluted with HEPES to final volume of 800 μL. All the setting parameters were the same as for size measurement, except an equilibration time of 60 sec. Samples were measured three times with 15 sub runs, and the zeta potential was calculated by the Smuchowski equation.

siRNA Gel Shift Assay

A 2.5% agarose gel was prepared with TBE buffer (trizma base 10.8 g, boric acid 5.5 g, disodium EDTA 0.75 g, and 1 L of water). 20 μL of siRNA polyplexes in HBG at concentration of 25 μg/mL was mixed with loading buffer (6 mL of glycerol, 1.2 mL of 0.5 M EDTA, 2.8 mL of H₂O, 0.02 g of bromophenol blue) at ratio of 5:1 (V/V) followed by placing in the gel pockets. The gel electrophoresis was run at 100 V for 40 min in TBE buffer.

### Cell Culture

The adherent human cervix carcinoma cell line KB/eGFPLuc (stably expressing the enhanced green fluorescent protein/luciferase (eGFPLuc) fusion gene) [Dohmen et al. ACS Nano 2012] was cultured in Dulbecco's modified Eagles's medium (DMEM)-low glucose (1 g/L glucose) supplemented with 10% (v/V) fetal bovine serum (FBS), stable glutamine (4 mM), penicillin (100 U/mL), and streptomycin (100 μg/mL). The cells were kept at 37° C. and 5% CO₂ in an incubator with a relative humidity of 95%

### Gene Silencing Mediated by GFP-siRNA

The KB/eGFPLuc cells were seeded in 96-well-plate (5×10³ cells/well) 24 h prior to the experiment. The culture medium was replaced with 80 μL of fresh supplemented medium followed by siRNA polyplex transfection. The polyplexes were prepared at indicated N/P ratio with control siRNA (siCtrl) or GFP-siRNA (siGFP for targeting GFPLuc protein) at concentration of 25 μg/mL in HBG. The volumes of 10, 5, and 2.5 μL of siRNA polyplex solution were added to the corresponding wells in triplicate as well as HBG to reach a final volume of 100 μL per well. HBG buffer was used as negative control. After 48 h, the medium was removed and cells were treated with 100 μL of cell culture 0.5× lysis buffer (Promega, Mannheim, Germany) followed by 45 min incubation at RT. Luciferase activity was measured in the 35 μL cell lysate by a Centro LB 960 plate reader luminometer (Berthold Technologies, Bad Wildbad, Germany) using LAR buffer (20 mM glycylglycine; 1 mM MgCl2; 0.1 mM

EDTA (ethylenediaminetetraacetic acid); 3.3 mM DTT (dithiothreitol); 0.55 mM ATP (adenosine 5′-triphosphate); 0.27 mM coenzyme A, pH 8-8.5) supplemented with 5% (v/V) of a mixture of 10 mM luciferin and 29 mM glycylglycine. The relative light units (RLUs) were presented as percentage of the luciferase gene expression of the HBG buffer-treated cells.

### Results

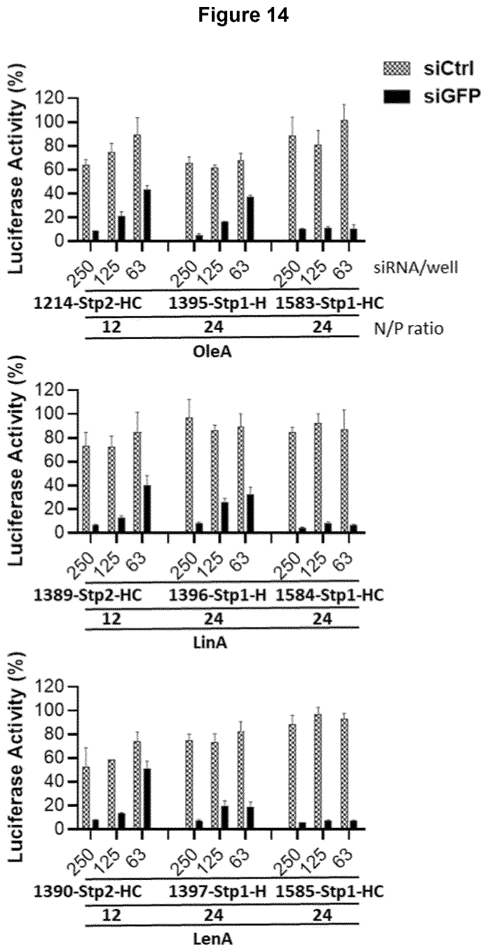

In a gene silencing assay using siRNA targeting GFP, the more polar structures #1214 (Stp2-HC) comprising OleA was compared to the respective non-polar structure #1395 and #1583, the same structure without the terminal cysteine (FIG. 14, top), as well as the same structures comprising LinA (#1389, #1396, #1584; FIG. 14, middle) and LenA (#1390, #1397, #1585; FIG. 14, bottom). siRNA polyplexes were formed with carriers at indicated N/P ratios in HBG (25 μg siRNA/mL). The size, PDI, and zeta potential were measured via DLS and ELS (n=3, mean±SD). The tested structures and the respective particle sizes and zeta potential are listed in Table 3 below:

TABLE 3
Particle size and zeta potential of siRNA polyplexes

| Oligo ID | Abbreviation | FA | N/P ratio | Size | PDI | Zeta |
|---|---|---|---|---|---|---|
| 1214 | Stp2-HC | OleA | 12 | 120.1 ± 2.1 | 0.37 ± 0.02 | 28.2 ± 2.1 |
| 1395 | Stp1-H | OleA | 24 | 105.4 ± 2.1 | 0.23 ± 0.02 | 23.2 ± 2.7 |
| 1583 | Stp1-HC | OleA | 24 | 125.2 ± 0.4 | 0.27 ± 0.00 | 34.0 ± 0.8 |
| 1389 | Stp2-HC | LinA | 12 | 182.1 ± 14.8 | 0.33 ± 0.03 | 29.6 ± 2.1 |
| 1396 | Stp1-H | LinA | 24 | 110.5 ± 0.6 | 0.24 ± 0.01 | 25.2 ± 1.2 |
| 1584 | Stp1-HC | LinA | 24 | 169.2 ± 0.9 | 0.18 ± 0.02 | 30.6 ± 1.5 |
| 1390 | Stp2-HC | LenA | 12 | 109.2 ± 1.2 | 0.23 ± 0.01 | 31.8 ± 1.1 |
| 1397 | Stp1-H | LenA | 24 | 99.8 ± 1.1 | 0.22 ± 0.01 | 37.3 ± 0.6 |
| 1585 | Stp1-HC | LenA | 24 | 95.3 ± 0.4 | 0.24 ± 0.01 | 39.4 ± 1.7 |

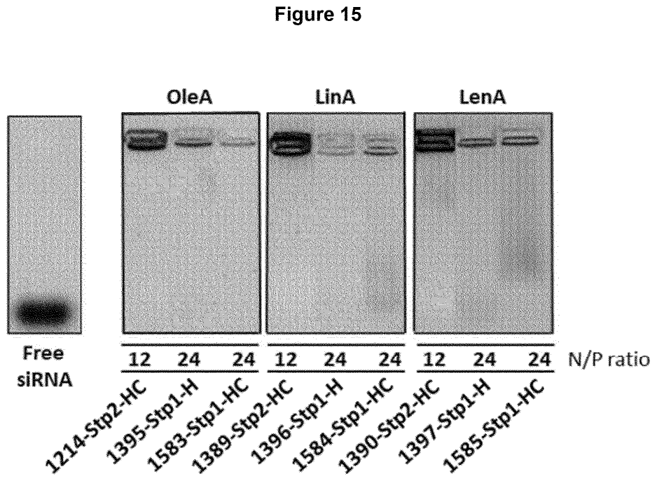

As may be taken from FIG. 14, particularly at low siRNA concentrations the more hydrophobic structures are more active in gene silencing, the terminal cysteine seems favorable. No gross change was observed with the use of the different fatty acids. In siRNA gel shift assays, all lipopeptides bound siRNA efficiently at the used N/P ratios (FIG. 15).

1. A nanoparticle for Cas protein/gRNA ribonucleoprotein (RNP) complex delivery comprising one or more Cas protein/gRNA RNP complex(es) as cargo;
and a carrier comprising a sequence-defined T-shape lipo-oligomer, comprising an artificial amino acid, wherein the artificial amino acid is an oligo(alkylamino) acid, and
wherein the T-shape lipo-oligomer comprises the sequence of formula I: 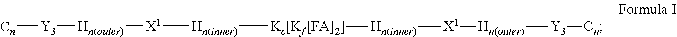
wherein
K_{c} is a central lysine;
K_{f} is a further lysine linked to the ε-amino group of K_{c};
FA is a fatty acid covalently linked to the ε- and α-amino group of K_{f},
C is cysteine and n=0 or 1, preferably n=0
Y₃ is a tyrosine tripeptide;
H is histidine and n₍ₒᵤₜₑᵣ₎ and n₍ᵢₙₙₑᵣ₎ are independently selected from 0, 1 or 2; and
X¹ is an oligo(alkylamino) acid of formula IIa: 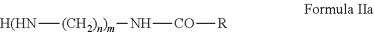
wherein n=2 or 3, alternating 2 and 3 or alternating 3 and 2; and m=3 or 4; and
wherein R is
—(CH₂)_{y}—CO₂H, wherein y=2, 3 or 4; 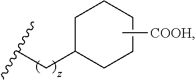
wherein z=0 or 1, preferably 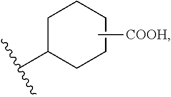
more preferably 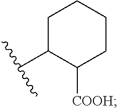 [SMILES: *C1CCCCC1C(=O)O]
—CH₂—CH(c-hexyl)-CH₂—CO₂H; or
—CH₂—NRₐ—CH₂—CO₂H, wherein Rₐ═H, CH₂—CF₃, or
wherein the T-shape lipo-oligomer comprises the sequence of formula III: 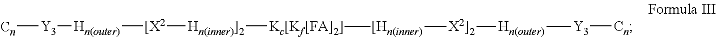
wherein
K_{c} is a central lysine;
K_{f} is a further lysine linked to the ε-amino group of K_{c};
FA is a fatty acid covalently linked to the ε- and α-amino group of Kᵣ,
C is cysteine and n=0 or 1, preferably n=1
Y₃ is a tyrosine tripeptide;
H is histidine and n₍ₒᵤₜₑᵣ₎ and n₍ᵢₙₙₑᵣ₎ are independently selected from 0 or 1; and
X² is an oligo(alkylamino) acid of formula IIa: 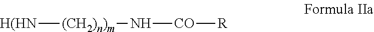
wherein n=2 or 3, alternating 2 and 3 or alternating 3 and 2; and m=3 or 4;
wherein R is
—(CH₂)_{y}—CO₂H, wherein y=3 or 4; 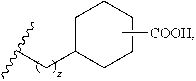
wherein z=0 or 1,
preferably 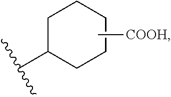
more preferably 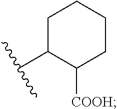 [SMILES: *C1CCCCC1C(=O)O]
—CH₂—CH(c-hexyl)-CH₂—CO₂H; or
—CH₂—NRₐ—CH₂—CO₂H, wherein Rₐ═CH₂—CF₃.

2. The nanoparticle of claim 1, wherein the two fatty acids are saturated or unsaturated C12 to C22 fatty acids, preferably the two fatty acids are selected from the group consisting of oleic acid (OleA), linoleic acid (LinA), linolenic acid (LenA) or hydroxy steric acid (OHSteA), preferably OleA, LinA or OHSteA, more preferably LinA or OHSteA.

3. The nanoparticle of claim 1, wherein the oligo(alkylamino) acid is a tetraethylenepentamine or a triethylenetetramine of formula IIb: 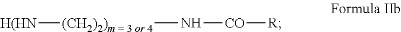
preferably a tetraethylenepentamine.

4. The nanoparticle of claim 1, wherein the T-shape lipo-oligomer comprises the sequence of formula I: 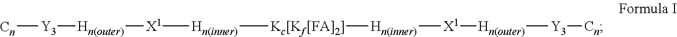
(a) wherein
C is cysteine and n=0 or 1, preferably n=0
Y₃ is a tyrosine tripeptide;
H is histidine and n₍ₒᵤₜₑᵣ₎ and n₍ᵢₙₙₑᵣ₎ are 1; or
(b) wherein
C is cysteine and n=0
Y₃ is a tyrosine tripeptide;
H is histidine and n₍ₒᵤₜₑᵣ₎ and n₍ᵢₙₙₑᵣ₎ are 0; or
wherein the T-shape lipo-oligomer comprises the sequence of formula III: 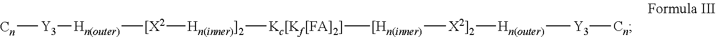
(a) wherein
C is cysteine and n=1
Y₃ is a tyrosine tripeptide;
H is histidine and n₍ₒᵤₜₑᵣ₎ and n₍ᵢₙₙₑᵣ₎ are 1; or
(b) wherein
C is cysteine and n=1
Y₃ is a tyrosine tripeptide;
H is histidine and n₍ₒᵤₜₑᵣ₎ and n₍ᵢₙₙₑᵣ₎ are 0.

5. The nanoparticle of claim 1, wherein the sequence-defined T-shape lipo-oligomer
(a) further comprises a terminal functional group at the N-terminus or the C-terminus of the T-shape lipo-oligomer, wherein the function group is (i) an azido group, or (ii) a thiol group, provided that the sequence of formula I does not comprise a cysteine; and/or
(b) further comprises a terminal azido group and a targeting ligand is coupled to the azido group, preferably wherein the targeting ligand is coupled to the azido group via click chemistry, more preferably click chemistry with a dibenzocylcooctyne-coupled targeting ligand.

6. The nanoparticle of claim 1, wherein the RNP complex comprises Cas protein/gRNA at a ratio of about 1:1 to about 1:2 and/or wherein the Cas protein/gRNA RNP complex and the sequence-defined T-shape lipo-oligomer are mixed at a lipo-oligomer nitrogen (N) to gRNA phosphate (P) ratio (N/P ratio) of about 1:12 to 1:30, preferably about 1:20 to 1:30, more preferably of about 1:22 to 1:26.

7. The nanoparticle of claim 1, wherein
(a) the Cas protein is a Cas9, a Cas12, a Cas13 protein or an engineered variant thereof;
(b) the Cas protein is a base editor or a prime editor;
(c) the one or more Cas protein/gRNA RNP complex(es) is/are Cas9/gRNA RNP complex(es); and/or
(d) wherein the nanoparticle comprises two or more Cas protein/gRNA RNP complexes as cargo and wherein the two or more Cas protein/gRNA RNP complexes comprise two or more different gRNAs targeting the same or different genes, preferably different genes; and/or
(e) wherein the nanoparticle comprises one or more Cas protein/gRNA RNP complexes and a donor DNA.

8. A nanoparticle for siRNA or phosphorodiamidate morpholino oligomers (PMO) delivery comprising one or more siRNA(s) or PMO(s) as cargo; and a carrier comprising a sequence-defined T-shape lipo-oligomer, comprising an artificial amino acid, wherein the artificial amino acid is an oligo(alkylamino) acid, and
wherein the T-shape lipo-oligomer comprises the sequence of formula I: 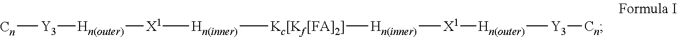
wherein
K_{c} is a central lysine;
K_{f} is a further lysine linked to the ε-amino group of K_{c};
FA is a fatty acid covalently linked to the ε- and α-amino group of K_{f};
C is cysteine and n=0 or 1, preferably n=0
Y₃ is a tyrosine tripeptide;
H is histidine and n₍ₒᵤₜₑᵣ₎ and n₍ᵢₙₙₑᵣ₎ are independently selected from 0, 1 or 2; and
X¹ is an oligo(alkylamino) acid of formula IIIa: 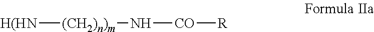
wherein n=2 or 3, alternating 2 and 3 or alternating 3 and 2; and m=3 or 4; and
wherein R is
—(CH₂)_{y}—CO₂H, wherein y=2, 3 or 4; 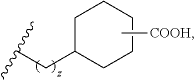
wherein z=0 or 1,
preferably 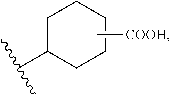
more preferably 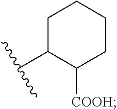 [SMILES: *C1CCCCC1C(=O)O]
—CH₂—CH(c-hexyl)-CH₂—CO₂H; or
—CH₂—NRₐ—CH₂—CO₂H, wherein Rₐ═H, CH₂—CF₃, and
wherein the two fatty acids are saturated or unsaturated C12 to C22 fatty acids, preferably the two fatty acids are selected from the group consisting of oleic acid (OleA), linoleic acid (LinA), linolenic acid (LenA) or hydroxy steric acid (OHSteA), preferably OleA, LinA or OHSteA, more preferably LinA or OHSteA.

9. A conjugate comprising a phosphorodiamidate morpholino oligomer (PMO) covalently linked to a carrier comprising a sequence-defined T-shape lipo-oligomer, comprising an artificial amino acid, wherein the artificial amino acid is an oligo(alkylamino) acid, and
wherein the T-shape lipo-oligomer comprises the sequence of formula I: 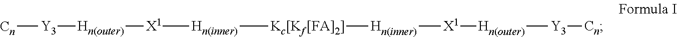
wherein
K_{c} is a central lysine;
K_{f} is a further lysine linked to the ε-amino group of K_{c};
FA is a fatty acid covalently linked to the ε- and α-amino group of K_{f};
C is cysteine and n=0 or 1, preferably n=0
Y₃ is a tyrosine tripeptide;
H is histidine and n₍ₒᵤₜₑᵣ₎ and n₍ᵢₙₙₑᵣ₎ are independently selected from 0, 1 or 2; and
X¹ is an oligo(alkylamino) acid of formula IIIa: 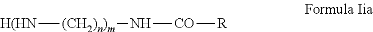
wherein n=2 or 3, alternating 2 and 3 or alternating 3 and 2; and m=3 or 4; and
wherein R is
—(CH₂)_{y}—CO₂H, wherein y=2, 3 or 4; 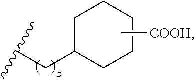
wherein z=0 or 1,
preferably 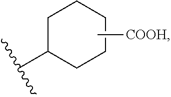
more preferably 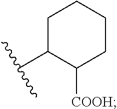 [SMILES: *C1CCCCC1C(=O)O]
—CH₂—CH(c-hexyl)-CH₂—CO₂H; or
—CH₂—NRₐ—CH₂—CO₂H, wherein Rₐ═H, CH₂—CF₃, and
wherein the two fatty acids are saturated or unsaturated C12 to C22 fatty acids, preferably the two fatty acids are selected from the group consisting of oleic acid (OleA), linoleic acid (LinA), linolenic acid (LenA) or hydroxy steric acid (OHSteA), preferably OleA, LinA or OHSteA, more preferably LinA or OHSteA.

10. A method of therapy comprising administering the nanoparticle of claim 1 to a subject.

11. A method of treating a subject with a disease, the method comprising administering to the subject the nanoparticle of claim 1, wherein the disease is selected from the group consisting of cancer, a genetic disease, an infectious disease, a cardiovascular disease, a metabolic disease, a neurodegenerative or neuromuscular disease, a hematological disease, a hereditary eye disease, and an autoimmune disease.

12. An in vitro method for transfecting mammalian cells with one or more Cas protein/gRNA complex, the method comprising contacting a mammalian cell in vitro with the nanoparticle of claim 1.

13. (canceled)

14. The in vitro method of claim 12, wherein the artificial amino acid is an oligo(alkylamino) acid selected from the group consisting of 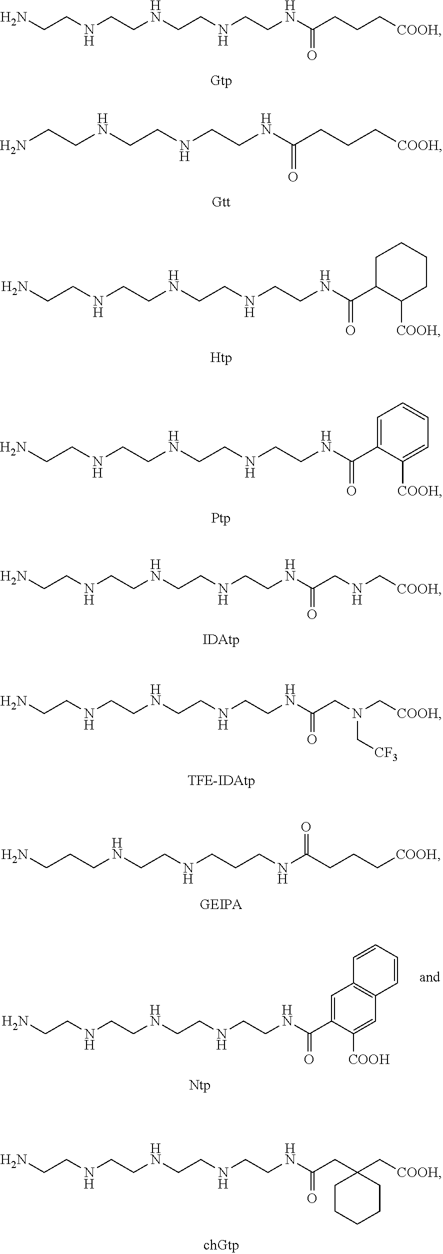
preferably, wherein the oligo(alkylamino) acid is selected from the group consisting of Gtp, Gtt, Htp, TFE-IDAtp, GEIPA, and chGtp, more preferably Htp, TFE-IDAtp and chGtp.

15. An artificial amino acid, wherein the artificial amino acid is an oligo(alkylamino) acid selected from the group consisting of the following formulas: 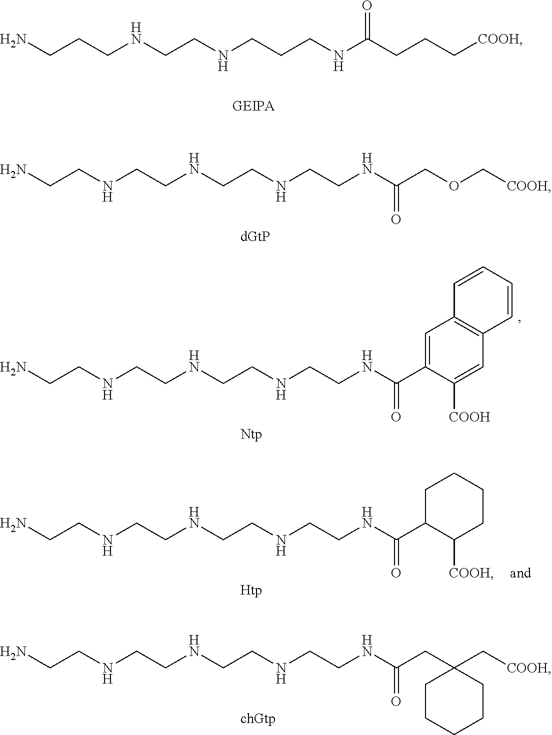 [SMILES: NCCCNCCNCCCNC(=O)CCCC(=O)O ; NCCNCCNCCNCCNC(=O)COCC(=O)O ; NCCNCCNCCNCCNC(=O)c1cc2ccccc2cc1C(=O)O ; NCCNCCNCCNCCNC(=O)C1CCCCC1C(=O)O ; NCCNCCNCCNCCNC(=O)CC1(CC(=O)O)CCCCC1]
in its protected and unprotected form.

16. A method for cellular delivery comprising use of the artificial amino acid of claim 15 for cellular delivery of a Cas protein/gRNA ribonucleoprotein (RNP) complex into a target cell.

17. An in vitro method for transfecting mammalian cells with one or more PMO, the method comprising contacting a mammalian cell in vitro with the conjugate of claim 9.

18. An in vitro method for transfecting mammalian cells with one or more siRNA or PMO, the method comprising contacting a mammalian cell in vitro with the nanoparticle of claim 8.
